(19) United States
(12) Patent Application Publication
(10) Pub. No.: US 2007/0191372 A1
(43) Pub. Date: Aug. 16, 2007

(54) Dna-targeted benzotriazine 1,4-dioxides and their use in cancer therapy

(75) Inventors: J. Martin Brown, Redwood City, CA (US); William Alexander Denny, Auckland (NZ); Michael Patrick Hay, Auckland (NZ); Kevin Owen Hicks, Auckland (NZ); Swarnalatha Akuratiya Gamage, Auckland (NZ); Frederik Bastiaan Pruijn, Auckland (NZ); William Robert Wilson, Waiuku (NZ)

Correspondence Address: NIXON & VANDERHYE, PC, 901 NORTH GLEBE ROAD, 11TH FLOOR, ARLINGTON, VA, 22203, US

(73) Assignees: Auckland Uniservices Limited, Auckland (NZ); The Board of Trustees of the Leland Stanford Junior University, Palo Alto, CA (US)

(21) Appl. No.: 10/528,156

(22) PCT Filed: Sep. 17, 2003
(86) PCT No.: PCT/NZ03/00210

§ 371 Date: Aug. 17, 2005

(30) Foreign Application Priority Data
Sep. 17, 2002 (NZ) 521436

(51) Int. Cl. A61K 31/53

(52) U.S. Cl. 514/243; 544/183

(57) ABSTRACT

The present invention relates to DNA-targeted 1,2,4-benzotriazine-1,4-dioxides and related analogues, to their preparation, and to their use as hypoxia-selective drugs and radiosensitizers for cancer therapy, both alone or in combination with radiation and/or other anticancer drugs.

### REFERENCE TO GOVERNMENT CONTRACT

The invention described herein was made in the course or work under grant or contract from the United States Department of Health and Human Services. The United States Government has certain rights to this invention.

### TECHNICAL FIELD

The present invention relates to DNA-targeted 1,2,4-benzotriazine-1,4-dioxides and related analogues, to their preparation, and to their use as hypoxia-selective drugs and radiosensitizers for cancer therapy, both alone or in combination with radiation and/or other anticancer drugs.

### BACKGROUND TO THE INTENTION

It has been established that many human tumors contain a significant hypoxic fraction of cells (Kennedy et al., Int. J. Radiat. Oncol. Biol. Phys., 1997, 37, 897-905; Movsas et al., Urology, 1999, 53, 11-18). The presence of hypoxic cells arises because of chaotic growth and an inefficient microvasculature system within the tumor, which leads to large intercapillary distances and variable blood flow. Reduction of oxygen tension in tumors leads to radioresistence. This reduction of oxygen tension causes up to a three-fold increase in radiation dose being required to kill anoxic tumor cells. A link has been identified between the presence of tumor hypoxia and failure of local control by radiation therapy (Brizel et al., Radiother. & Oncol., 1999, 53, 113-117). This phenomenon of tumor hypoxia has been exploited in the development of a class of anticancer agents termed ‘bioreductive drugs’ (Brown et al., Semin. Radiat. Oncol., 1966, 6, 22-36; Denny et al., Br. J. Cancer, 1996, 74 (Suppl. XXVII) 32-38; Stratford & Workman, Anti-Cancer Drug Des., 1998, 13, 519-528). These agents are selectively active against hypoxic cells in tumors by targeting the DNA of these cells. The agents cause irreversible damage to the DNA of the tumor cells, thereby causing the destruction and breakdown of the tumor.

Tirapazamine (TPZ, 3-amino-1,2,4-benzotriazine 1,4-dioxide) is a bioreductive agent Wilson et al., Anti-Cancer Drug Des., 1998, 13, 575-592; Lee et al., WO 9104028, April 1991) and is undergoing clinical trials in combination with radiotherapy and various chemotherapeutics, notably cisplatin (Denny & Wilson, Exp. Opin. Invest. Drugs, 2000, 9, 2889-2901).

TPZ is activated by one electron reductases (Patterson et al., Anti-Cancer Drug Des. 1998 13, 541-573; Denny & Wilson, Exp. Opin. Invest. Drugs, 2000, 9, 2889-2901) to form a radical anion (Scheme A). This TPZ radical anion may be oxidized back to TPZ by molecular oxygen under aerobic conditions.

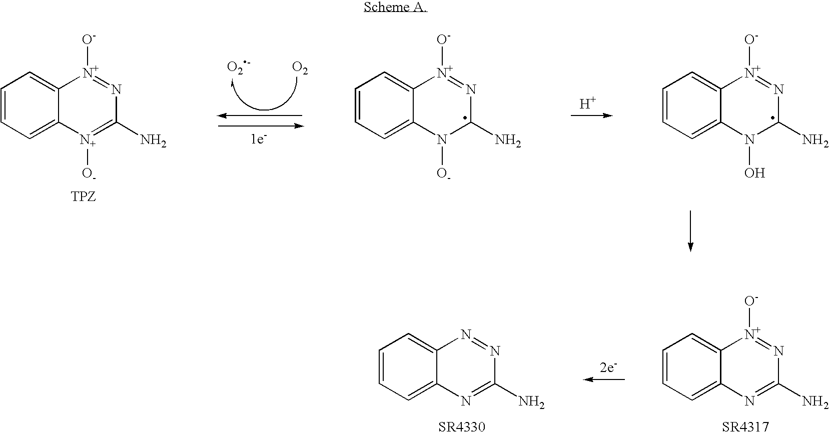

Under hypoxic conditions the radical or species ultimately derived from TPZ can interact with DNA, although the exact mechanism is unclear (Jones et al., Cancer Res., 1996, 56, 1584-1590; Daniels et al., Chem. Res. Toxicol., 1998, 11, 1254-1257; Hwang et al., Biochem., 1999, 38, 14248-14255). TPZ causes DNA double-strand breaks under anoxic conditions (Jones et al., Cancer Res., 1996, 56, 1584-1590) and these results correlate with cytotoxicity (Dorie et al., Neoplasia, 1999, 1, 461-467). Reversible one-electron reduction of TPZ that gives rise to a reactive radical species that is thought to be the basis for selective toxicity to hypoxic cells. Two electron reduction of TPZ or further reduction of the TPZ radical produces the metabolite 1-oxide (SR 4317) and further reduction gives the nor-oxide (SR 4330) (Baker et al., Cancer Res., 1988, 48, 5947-5952; Laderoute & Rauth, Biochem Pharmacol., 1986, 35, 3417-3420) (Scheme A). The metabolites (SR 4317) and (SR 4330) are both inactive under aerobic or hypoxic conditions.

It is also known that reactive species can be effectively targeted to DNA by attachment to DNA-affinic carriers. Thus, the intrinsic cytotoxicities and in vivo potencies of aniline mustards can be significantly increased (up to 100-fold), and the usual dependence of cytotoxicity on mustard reactivity lowered, by targeting to DNA via a 9-aminoacridine carrier (Gourdie et al., J. Med. Chem., 1990, 33, 1177-1185). DNA alkylation patterns can also be significantly altered (Prakash et al., Biochem., 1990, 29, 9799-9807; Boritzki et al., Chem. Res. Toxicol., 1994, 7, 41-46). Alkylation of DNA by DNA-targeted compounds is more rapid than with the corresponding untargeted compounds (O'Connor et al., Chem.-Biol. Int., 1992, 85, 1-14). However, the extent of DNA binding needs to be carefully adjusted to achieve effective targeting without significantly compromising the transport/diffusion properties (Hicks et al., J. Pharmacol. Exp. Therapeut. 2001, 297, 1088-1098; Hicks et. al., Brit. J. Cancer. 1997, 76, 894-903). Binding ability can be varied by alteration of both the chromophore and substituents on the DNA targeted compound (Palmer et al., J. Med. Chem., 1988, 31, 707-712).

It is an object of the present invention to utilize DNA-affinic carriers in combination with benzotriazine 1,4-dioxides to target DNA for cancer therapy purposes, or to at least provide the public with a useful choice.

### DISCLOSURE OF THE INVENTION

In a first aspect, the present invention provides a compound of Formula I, 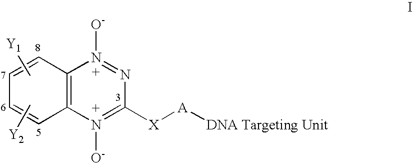
wherein
Y₁ and Y₂ at one or more of the available carbons 5-8 on the benzo ring: are each independently selected from the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;

wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR₂² or N(OH)R² wherein each R² is independently selected from C₁₋₄ alkyl, C₂₋₄ alkenyl, OH, NO₂, NH₂, CF₃, CN, CO₂H or SH, and
   wherein X represents NH, NMe, CH₂, SO, SO₂, or O;
   A represents an optionally substituted C₁₋₁₂ alkyl group wherein the optional substituents are each independently selected from OH, OR, NH₂, NHR³, NR₂³, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH; and
   wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.,
   or a pharmacologically acceptable salt thereof.

The definition of the DNA targeting unit above refers to double-stranded random-sequence DNA. An example of such double-stranded random-sequence DNA is DNA extracted from calf thymus.

A preferred compound of Formula I is one in which X is NH or CH₂.

A further preferred compound of Formula I is one in which Y₁ and Y₂ each represent H.

A further preferred compound of Formula I is one in which Y₁ represents OMe

A preferred embodiment of Formula I are compounds wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

A further preferred embodiment of Formula I are compounds wherein the DNA-targeting unit is selected from one of formulae II-XVI, 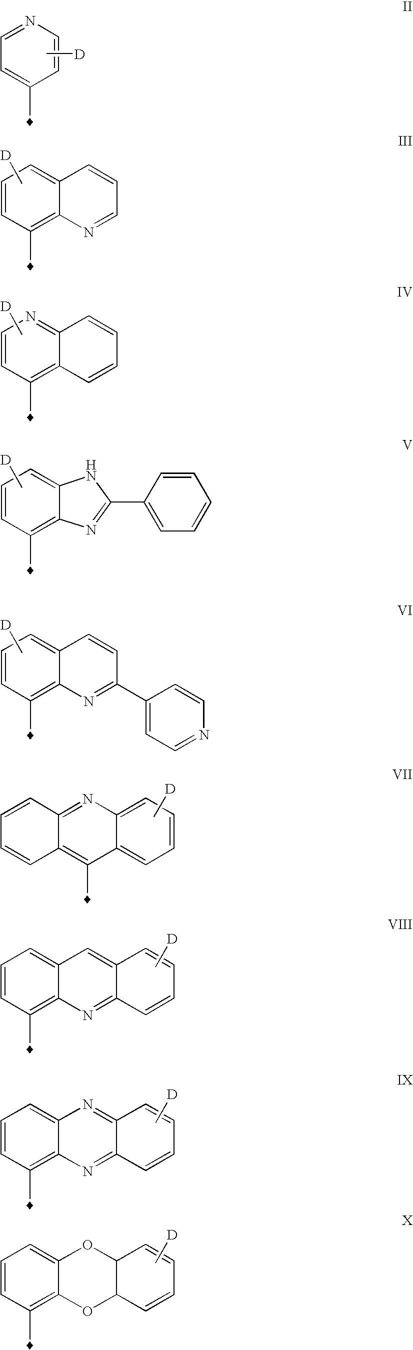 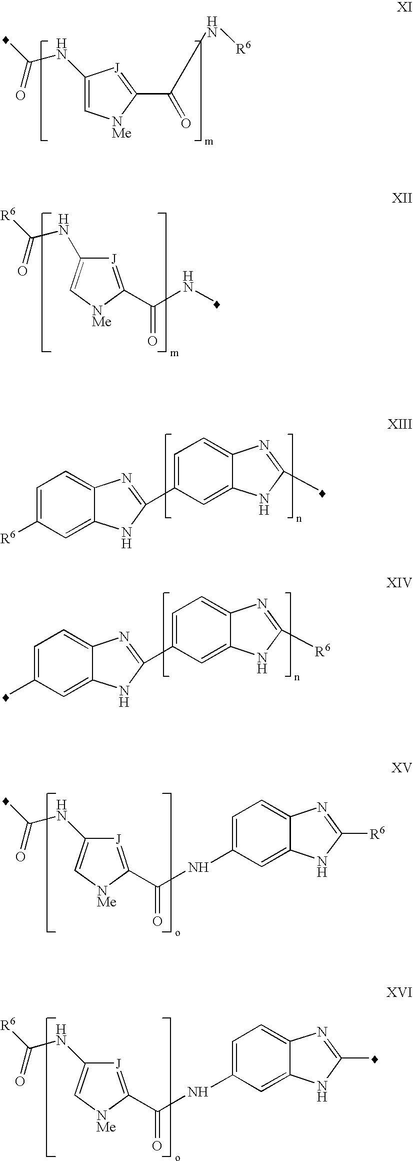
wherein in structures XI-XVI R⁶ is independently selected from an optionally substituted C₁₋₆ alicyclic or an optionally substituted C₃₋₆ cyclic alkyl group, and wherein the optional substituents are each independently selected from; halo, OH, OR⁷NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;

R⁶ can also represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR₂⁸ or N(OH)R⁸ wherein each R⁸ is independently selected from C₁₋₄ alkyl, C₂₋₄ alkenyl, OH, NO₂, NH₂, CF₃, CN, CO₂H or SH;
   D represents up to four of the following groups as substituents at any available ring carbon position; H, R⁹, hydro r, alkoxy, halogen, NO₂, NH₂, NHR⁹, NR₂⁹, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl and morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR₂¹⁰ or N(OH)R¹⁰ wherein each R¹⁰ is independently selected from C₁₋₄ alkyl, C₂₋₄ alkenyl, OH, NO₂, NH₂, CF₃, CN, CO₂H or SH;
   and wherein any available ring carbon position of formulae II-XVI can also be optionally replaced by —N— when the valency and configuration of the formula allows, the point of attachment of formulae II-XVI to the A group defined above is represented by ♦; and
   wherein in formulae XI, XII, m is selected from 2, 3 or 4, and
   wherein in formulae M, XII, XV and XVI, J is selected from CH or N;
   and wherein in formulae XIII and XIV n is selected from 0, 1 or 2;
   and wherein in formulae XV and XVI o is selected from 1 and 2.

A preferred embodiment of formula I is one in which the DNA targeting unit is selected from one of formulae IV, V, VI, VII, VIII, or IX.

A preferred embodiment of formula I is one in which D of the DNA targeting unit of Formulae II-X is H or Me.

Further preferred compounds of formula I include the following

wherein X is NH—, Y₁ is H, Y₂ is H, A is —(CH₂)₆NH—, the DNA targeting unit represents formula VII and D is H;

wherein X is NH—, Y₁ is H, Y₂ is H, A is —(CH₂)₃NH(CH₂)₃NHCO—, the DNA targeting unit represents formula VIII and D is H:

wherein X is NH—, Y₁ is H, Y₂ is H, A is —(CH₂)₂NH(CH₂)₂NHCO—, the DNA targeting unit represents formula VIII and D is H:

wherein X is NH—, Y₁ is H, Y₂ is H, A is —(CH₂)₃NMe(CH₂)₃NHCO—, the DNA targeting unit represents formula VIII and D is H;

wherein X is NH—, Y₁ is H, Y₂ is H, A is —(CH₂)₃NMe(CH₂)₃NHCO—, the DNA targeting unit represents formula IV and D is H;

wherein X is NH—, Y₁ is H, Y₂ is H, A is —(CH₂)₃NMe(CH₂)₃NHCO—, the DNA targeting unit represents formula VI and D is H;

wherein X is NH—, Y₁ is H, Y₂ is H, A is —(CH₂)₃NMe(CH₂)₃NHCO—, the DNA targeting unit represents formula VIII and D is Me;

wherein X is NH—, Y₁ is H, Y₂ is H, A is —(CH₂)₃NMe(CH₂)₃NHCO—, the DNA targeting unit represents formula IX and D is Me;

wherein X is NH—, Y₁ is 7-MeOCH₂CH₂O—, Y₂ is H, A is —(CH₂)₃NMe(CH₂)₃NHCO—, the DNA targeting unit represents formula VIII and D is H;

wherein X is CH₂—, Y₁ is H, Y₂ is H, A is —(CH₂)₂NMe(CH₂)₃NHCO—, the DNA targeting unit represents formula VIII and D is H;

wherein X is NH—, Y₁ is H, Y₂ is H, A is —(CH₂)₂NMe(CH₂)₃NHCO—, the DNA targeting unit represents formula XI and D is H;

wherein X is NH—, Y₁ is 7-Me, Y₂ is H, A is —(CH₂)₃NMeH(CH₂)₃NHCO—, the DNA targeting unit represents formula VIII and D is H;

wherein X is NH—, Y₁ is 7-Me, Y₂ is H, A is —(CH₂)₃NMe(CH₂)₃NHCO—, the DNA targeting unit represents formula VI and D is H;

wherein X is NH—, Y₁ is 6-Me, Y₂ is H, A is —(CH₂)₃NMe(CH₂)₃NHCO—, the DNA targeting unit represents formula VIII and D is H;

wherein X is NH—, Y₁ is 6-Me, Y₂ is H, A is —(CH₂)₃NMe(CH₂)₃NHCO—, the DNA targeting unit represents formula VI and D is H;

wherein X is NH—, Y₁ is H, Y₂ is H, A is —(CH₂)₂NMe(CH₂)₂NHCO—, the DNA targeting unit represents formula VIII and D is H;

wherein X is NH—, Y₁ is H, Y₂ is H, A is —(CH₂)₂NMe(CH₂)₂NHCO—, the DNA targeting unit represents formula VI and D is H;

wherein X is NH—, Y₁ is H, Y₂ is H, A is —(CH₂)₂NMe(CH₂)₂NHCO—, the DNA targeting unit represents formula XI and D is Me;

wherein X is NH—, Y₁ is H, Y₂ is H, A is —(CH₂)₂NMe(CH₂)₂NHCO—, the DNA targeting unit represents formula VIII and D is Me;

wherein X is NH—, Y₁ is H, Y₂ is H, A is —(CH₂)₂NH(CH₂)₂NHCO—, the DNA targeting unit represents formula VI and D is H; and

wherein X is NH—, Y₁ is H, Y₂ is H, A is —(CH₂)₂NH(CH₂)₂NHCO—, the DNA targeting unit represents formula VIII and D is Me.

In a second aspect, the present invention provides a compound of Formula I′, 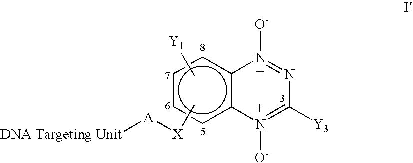
wherein
Y₁ represents at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
Y₃ is selected from the following groups halo, H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl and morpholino;
wherein each R of groups Y₁ and Y₃ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted
C₁₋₄ alkyl or an optionally substituted C₂₋₄ alkenyl group and wherein the optional substituents are each independently selected from OH, OR, NH₂, NHR², NR₂² or N(OH)R² wherein each R² is independently selected from C₁₋₄ alkyl C₂₋₄ alkenyl, OH, NO₂, NH₂, CF₃, CN, CO₂H or SH, and
wherein X represents NH, NMe, CH₂, SO, SO₂, or O;
A represents an optionally substituted C₁₋₁₂ alkyl group wherein the optional substituents are each independently selected from OH, OR, NH₂, NHR³, NR₂³ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₂₋₁₂ alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH; and
wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C.,
or a pharmacologically acceptable salt thereof.

The definition of the DNA targeting unit above refers to double-stranded random-sequence DNA. An example of such double-stranded random-sequence DNA is DNA extracted from calf thymus.

A preferred compound of Formula I′ is one in which X is O, NH or CH₂.

A further preferred compound of Formula I′ is one in which Y₁ represents H.

A preferred embodiment of Formula I′ are compounds wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

A further preferred embodiment of Formula I′ are compounds wherein the DNA-targeting unit is selected from one of formulae II-XVI, 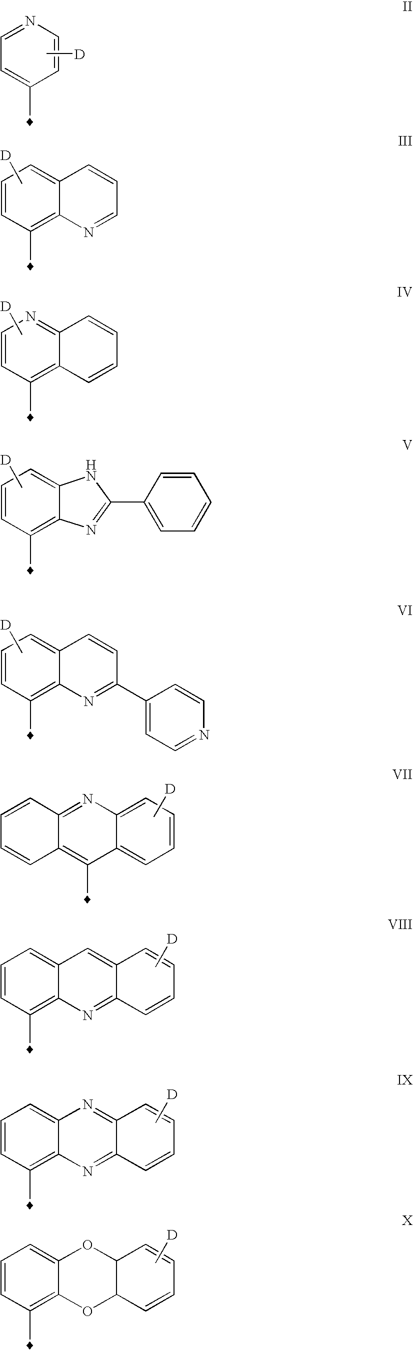 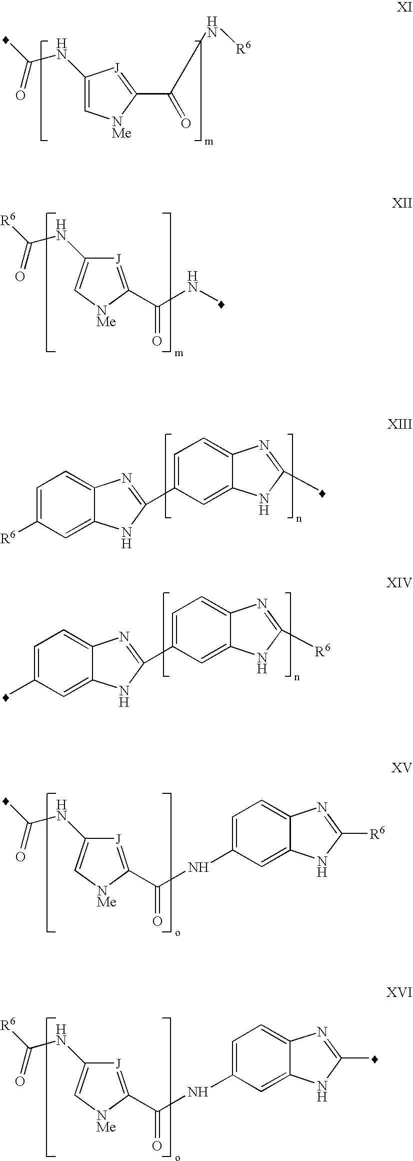
wherein in structures XI-XVI R⁶ is independently selected from an optionally substituted C₁₋₆ alicyclic or an optionally substituted C₃₋₆ cyclic alkyl group, and wherein the optional substituents are each independently selected from: halo, OH, OR⁷NO₂, NH₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;

R⁶ can also be represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;

wherein each R⁷ is independently selected from an optionally substituted C₁₋₄ alkyl or an optionally substituted C₂₋₄ alkenyl croup and wherein the optional substituents are each independently selected from OH, OR⁸, NH₂, NHR⁸, NR₂⁸ or N(OH)R⁹⁸ wherein each R⁸ is independently selected from C₁₋₄ alkyl, C₂₋₄ alkenyl, OH, NO₂, NH₂, CF₃, CN, CO₂H or SH;

D represents up to four of the following groups as substituents at any available ring carbon position; H, R⁹, hydroxy, alkoxy, halogen, NO₂, NH₂, NHR⁹, NR₂⁹, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl and morpholino, wherein each R⁹ independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR₂¹⁰ or N(OH)R¹⁰ wherein each R¹⁰ is independently selected from C₁₋₄ alkyl, C₂₋₄ alkenyl, OH, NO₂, NH₂, CF₃, CN, CO₂H or SH;

and wherein any available ring carbon position of formulae II-XVI can also be optionally replaced by —N— when the valency and configuration of the formula allows, the point of attachment of formulae II-XVI to the A group defined above is represented by ♦; and

wherein in formulae XI and XII, m is selected from 2, 3 or 4,

and wherein in formulae XI, XII, XV or XVI J is selected from CH or N; and

wherein in formulae XIII and XIV n is selected from 0, 1 or 2, and

wherein in formulae XV and XVI o is selected from 1 or 2.

A preferred embodiment of formula I′ is one in which the DNA targeting unit is selected from one of formulae III-IX.

A preferred embodiment of formula I′ is one in which D of the DNA targeting unit of Formulae II-X is H or Me.

Preferred compounds of formula I′ include the following

wherein X is O—, Y is H, A is —(CH₂)₃NH(CH₂)₃NHCO—, the DNA targeting unit represents formula VI and D is H;

wherein X is O—, Y is H, A is —(CH₂)₃NMe(CH₂)₃NHCO—, the DNA targeting unit

represents formula VI and D is H;

wherein X is O—, Y is H, A is —(CH₂)₂NH(CH₂)₂NHCO—, the DNA targeting unit represents formula VI and D is H;

wherein X is O—, Y is H, A is —(CH₂)₂NMe(CH₂)₂NHCO—, the DNA targeting unit represents formula VI and D is H;

wherein X is O—, Y is H, A is —(CH₂)₃NH(CH₂)₃NHCO—, the DNA targeting unit represents formula VIII and D is H;

wherein X is O—, Y is H, A is —(CH₂)₃NMe(CH₂)₃NHCO—, the DNA targeting unit represents formula VIII and D is H;

wherein X is O—, Y is H, A is —(CH₂)₂NH(CH₂)₂NHCO—, the DNA targeting unit represents formula VIII and D is H;

wherein X is O—, Y is H, A is —(CH₂)₂NMe(CH₂)₂NHCO—, the DNA targeting unit represents formula VIII and D is H;

wherein X is O—, Y is H, A is —(CH₂)₃NH(CH₂)₃NHCO—, the DNA targeting unit represents formula VIII and D is Me;

wherein X is O—, Y is H, A is —(CH₂)₃NMe(CH₂)₃NHCO—, the DNA targeting unit represents formula VIII and D is Me;

wherein X is O—, Y is H, A is —(CH₂)₂NH(CH₂)₂NHCO—, the DNA targeting unit represents formula VIII and D is Me;

wherein X is O—, Y is H, A is —(CH₂)₂NMe(CH₂)₂NHCO—, the DNA targeting unit represents formula VIII and D is Me;

wherein X is O—, Y is H, A is —(CH₂)₃NH(CH₂)₃NHCO—, the DNA targeting unit represents formula IX and D is Me.

wherein X is O—, Y is H, A is —(CH₂)₃NMe(CH₂)₃NHCO— the DNA targeting unit represents formula IX and D is Me; and wherein X is O—, Y is H, A is —(CH₂)₂NH(CH₂)₂NHCO—, the DNA targeting unit represents formula IX and D is Me;

wherein X is O—, Y is H, A is —(CH₂)₃NMe(CH₂, NHCO—, the DNA targeting unit represents formula IX and D is Me;

In a third aspect the invention provides for the use in a method of therapy for treating cancers including the step of administering a compound of Formula I as defined above or a compound of Formula I′ as defined above or a mixture thereof in a therapeutically effective amount to tumour cells in a subject

Preferably the tumour cells are in a hypoxic environment.

It is preferred that the method of therapy further includes the step of administering radiotherapy to the tumor cells before, during or after the administration of the compound of Formula I as defined above or a compound of Formula I′ as defined above or a mixture thereof to the tumour cells.

It is preferred that the method of therapy further includes the step of administering one or more chemotherapeutic agents to the tumor cells before, during or after the administration of the compound of Formula I as defined above or a compound of Formula I′ as defined above or a mixture thereof to the tumour cells.

While these compounds will typically be used in cancer therapy of human subjects, they can be used to target tumor cells in other warm blooded animal subjects such as other primates, farm animals such as cattle, and sports animals and pets such as horses, dogs, and cats.

A “therapeutically effective amount”, is to be understood as an amount of a compound of Formula I as defined above or a compound of Formula I′ as defined above or a mixture thereof that is sufficient to show benefit to a patient. The actual amount, rate and time-course of administration, will depend on the nature and severity of the disease being treated. Prescription of treatment is within the responsibility of general practitioners and other medical doctors.

A hypoxic environment is to be understood as either an in vitro or in vivo environment having a poorer blood supply and lower oxygen tension than normal tissues.

It is to be understood that the compound of Formula I or Formula I′ can be administered alone or in combination with other chemotherapeutic agents or treatments, especially radiotherapy, either simultaneously or sequentially dependent upon the condition to be treated.

Preferred chemotherapeutic agents can be selected from:

Cisplatin or other platinum-based derivatives,

Temozolomide or other DNA methylating agents,

Cyclophosphamide or other DNA alkylating agents,

Doxorubicin, mitoxantrone, camptothecin or other topoisomerase inhibitors,

Methotrexate, gemcitabine or other antimetabolites.

In a fourth aspect of the present invention there is provided a pharmaceutical composition including a therapeutically effective amount of a compound of formula I or compound of formula I′ or a mixture thereof, a pharmaceutically, acceptable excipient, adjuvant, carrier, buffer or stabiliser.

The pharmaceutically acceptable excipient, adjuvant, carrier, buffer or stabiliser should be non-toxic and should not interfere with the efficacy of the active ingredient. The precise nature of the carrier or other material will depend on the route of administration, which can be oral, or by injection, such as cutaneous, subcutaneous, or intravenous injection.

Pharmaceutical compositions for oral administration can be in tablet, capsule, powder or liquid form. A tablet may comprise a solid carrier or an adjuvent. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such as gelatin.

For intravenous, cutaneous or subcutaneous injection, the active ingredient will be in the form of a parenterally acceptable aqueous solution which is pyrogen-free and has a suitable pH, isotonicity and stability. Those of relevant skill in the art are well able to prepare suitable solutions using, for example, isotonic vehicles such as Sodium Chloride injection, Ringer's injection, Lactated Ringer's injection. Preservatives, stabilisers, buffers antioxidants and/or other additives may be included as required.

In a fifth aspect of the present invention there is provided a method of making a compound of formula XVII 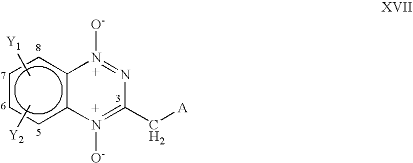
wherein
Y₁ and Y₂ at one or more of the available carbons 5-8 on the benzo ring: are each independently selected from the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
wherein each R is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring, system which are each independently selected from O, N or S; wherein each R¹ is independently selected from an optionally substituted C₁₋₄ alkyl or an optionally substituted C₂₋₄ alkenyl group and wherein the optional substituents are each independently selected from OH, OR, NH₂, NHR², NR₂² or N(OH)R² wherein each R² is independently selected from C₁₋₄ alkyl, C₂₋₄ alkenyl, OH, NO₂, NH₂, CF₃, CN, CO₂H or SH, and

A represents an optionally substituted C₁₋₁₂ alkyl group wherein the optional substituents are each independently selected from OH, OR, NH₂, NHR³, NR₂³, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH;

or a pharmacologically acceptable salt thereof,

including the step of coupling a compound (a) using a palladium reagent to form compound (b) which can then be converted into a compound of XVII as defined above; 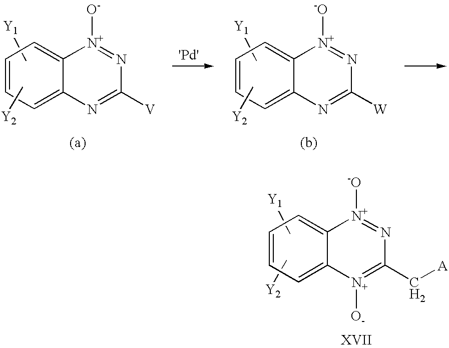
wherein in compound (a)
V is halogen which is selected from Cl, Br or I and Y₁, Y₂ are as defined above; and wherein in compound (b) Y₁, Y₂ are as defined above, W is selected from an optionally substituted
C₁₋₁₂alkyl, optionally substituted C₂₋₁₂alkenyl, and optionally substituted C₂₋₁₂alkynyl group, wherein the optional substituents is selected from halo, OH, OR⁶, NO₂, NH₂, NHR⁶, NR⁶R⁶, SH, SR⁶, imidazolyl, R⁶-piperazinyl, morpholino, SO₂R⁶, CF₃, CN, CO₂H, CO₂R⁶, CHO, COR⁶, CONH₂, CONHR⁶, CONR⁶R⁶, wherein each R⁶ is independently selected from an optionally substituted C₁₋₄ alkyl or an optionally substituted C₂₋₄ alkenyl group and wherein the optional substituents are each independently selected from OH, OR, NH₂, NHR⁷, NR₂⁷ or N(OH)R⁷ wherein each R⁷ is independently selected from C₁₋₄ alkyl, C₂₋₄ alkenyl, OH, NO₂, NH₂, CF₃, CN, CO₂H or SH.

In a sixth aspect of the present invention there is provided a method of making a compound of formula XVII′ 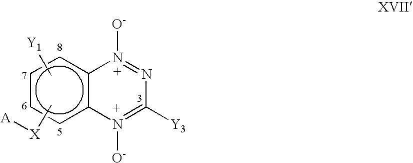
wherein Y₁ represents at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
Y₃ is selected from the following groups H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl and morpholino
wherein each R of groups Y₁ and Y₃ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from, halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₁, NHR²NR₂² or N(OH)R² wherein each R² is independently selected form C₁₋₄ alkyl, C₂₋₄ alkenyl, OH, NO₂, NH₂, CF₃, CN, CO₂H or SH, and
   wherein X represents NH, NMe, CH₂, SO, SO₂, or O;
   A represents an optionally substituted C₁₋₁₂ alkyl group wherein the optional substituents are each independently selected from OH, OR, NH₂, NHR³NR₂³ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl in is optionally interrupted by one or more heteroatom containing linkage moieties selected from O, NH, NR⁴, CONH, CONR⁴, NHCO, NR⁴CO, wherein each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH; and
   or a pharmacologically acceptable salt thereof;
   including the steps of coupling a compound (a) using a palladium reagent to form compound (b) which can then be converted into a compound of XVII′ as defined above; 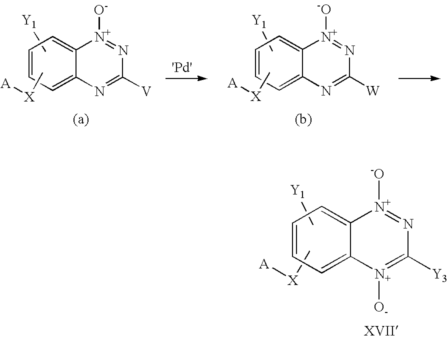
   wherein in compound (a)
   V is halogen which is selected from Cl, Br or I; Y₁, X and A are as defined above;
   and wherein in compound (b) Y₁, X and A are as defined above,
   W is selected from an optionally substituted
   C₁₋₂alkyl, optionally substituted C₂₋₁₂alkenyl, and optionally substituted C₂₋₁₂alkynyl group, wherein the optional substituents is selected from halo, OH, OR⁶, NO₂, NH₂, NHR⁶, NR⁶R⁶, SH, SR⁶, imidazolyl, R⁶-piperazinyl, morpholino, SO₂R⁶, CF₃, CN, CO₂H, CO₂R⁶, CHO, COR⁶, CONH₂, CONHR⁶, CONR⁶R⁶, wherein each R⁶ is independently selected from an optionally substituted C₁₋₄ alkyl or an optionally substituted C₂₋₄ alkenyl group and wherein the optional substituents are each independently selected from OH, OR, NH₂, NHR⁷, NR₂⁷ or N(OH)R⁷ wherein each R⁷ is independently selected from C₁₋₄ alkyl, C₂₋₄ alkenyl, OH, NO₂, NH₂, CF₃, CN, CO₂H or SH.

In a seventh aspect of the present invention there is provided a compound of formula XVIII 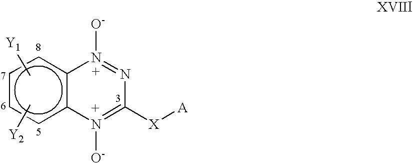
wherein
Y₁ and Y₂ at one or more of the available carbons 5-8 on the benzo ring: are each independently selected from the following groups: halo, H, R, OH, OR, NO₂, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
wherein each R is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;

wherein each R¹ is independently selected from an optionally substituted

C₁₋₄ alkyl or an optionally substituted C₂₋₄ alkenyl group and wherein the optional substituents are each independently selected from OH, OR, NH₂, NHR², NR₂² or N(OH)R² wherein each R² is independently selected from C₁₋₄ alkyl, C₂₋₄ alkenyl, OH, NO₂, NH₂, CF₃, CN, CO₂H or SH, and

wherein X represents NH, NMe, CH₂, SO, SO₂, or O;

A represents an optionally substituted C₁₋₁₂ alkyl group wherein the optional substituents are each independently selected from OH, OR, NH₂, NHR³, NR₂³, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH;

or a pharmacologically acceptable salt thereof.

In an eighth aspect of the present invention there is provided a compound of formula XVII′ 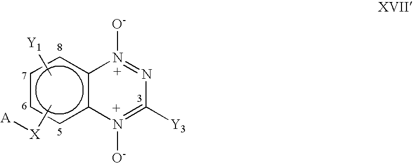
wherein
Y₁ represents at one or more of the available carbons 5-8 on the benzo ring the following groups:
halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
Y₃ is selected from the following groups H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl and morpholino
wherein each R of groups Y₁ and Y₃ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;

R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR₂² or N(OH)R² wherein each R² is independently selected from C₁₋₄ alkyl, C₂₋₄ alkenyl, OH, NO₂, NH₂, CF₃, CN, CO₂H or SH, and
   wherein X can represent NH, NMe, CH₂, SO, SO₂, or O;
   A can represent an optionally substituted C₁₋₁₂ alkyl group wherein the optional substituents are each independently selected from OH, OR, NH₂, NHR³, NR₂³ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR₂⁵ or N(OH)R⁵ wherein each R⁵ is independently selected from C₁₋₄ alkyl, C₂₋₄ alkenyl, OH, NO₂, NH₂, CF₃, CN, CO₂H or SH; and
   wherein X represents NH, NMe, CH₂, SO, SO₂, or O;
   or a pharmacologically acceptable salt thereof.

In a ninth aspect of the present invention there is provided a method of making a compound of Formula I defined above including the steps of
1 preparing a compound of Formula XVIII as defined above
2 coupling the compound of Formula XVIII with a DNA targeting agent as defined above to provide a compound of Formula I.

In a tenth aspect of the present invention there is provided a method of making a compound of Formula I′ defied above including the steps of
1 preparing a compound of Formula XVII′ as defined above
2 coupling the compound of Formula XVII′ with a DNA targeting agent as defined above to provide a compound of Formula I′.

It is to be recognised that certain compounds of the present invention may exist in one or more different enantiomeric or diastereomeric forms. It is to be understood that the enantiomeric or diasteriomeric forms are included in the above aspects of the invention.

The term halo or halogen group used throughout the specification is to be taken as meaning a fluoro, chloro, bromo or iodo group.

The term pharmaceutically acceptable salt used throughout the specification is to be taken as meaning any acid or base derived salts formed from hydrochloric, sulfuric, phosphoric, acetic, citric, oxalic, malonic, salicylic, malic, fumaric, succinic, ascorbic, maleic, methanesulfonic, isoethonic acids and the like and potassium carbonate sodium or potassium hydroxide ammonia, triethylamine, triethanolamine and the like.

Further aspects of the present invention will become apparent from the following description given by way of example only and with reference to the accompanying synthetic schemes.

### DETAILED DESCRIPTION OF THE INVENTION

Methods for Preparing Compounds of Formula I of the Invention.

3-Chloro-1,2,4-benzotriazine 1-oxide (3) was readily synthesised from 2-nitroaniline in 3 steps (50% yield) (Scheme 1). Preparation of the diamine 4 can be achieved as shown in Scheme 2. Coupling of chloride 3 with the monoprotected diamine 4, readily prepared in 85% yield from the 6-aminohexan-1-ol, gave carbamate 5 as illustrated in Scheme 3. Reaction of 5 with MCPBA in DCM gives 1,4-dioxide 6 in 39% yield and recovered starting material 5 (50%). This represents a departure from known methods (Lee et al, U.S. Pat. No. 5,616,584, April, 1997) that use trifluoroperacetic acid as the oxidant Cleavage of the 1,4-dioxide carbamate 6 with HCl in MeOH gave 1,4-dioxide 7 in good yield.

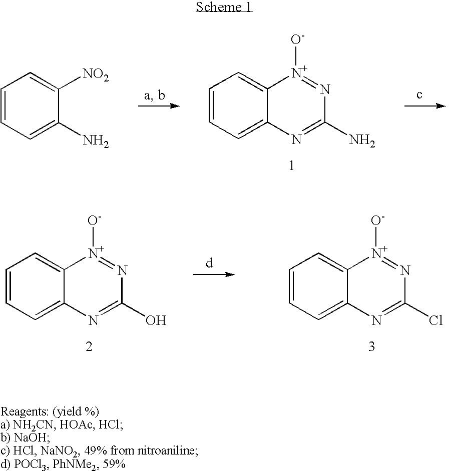

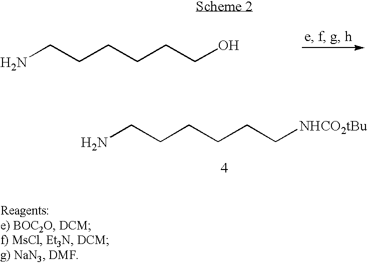

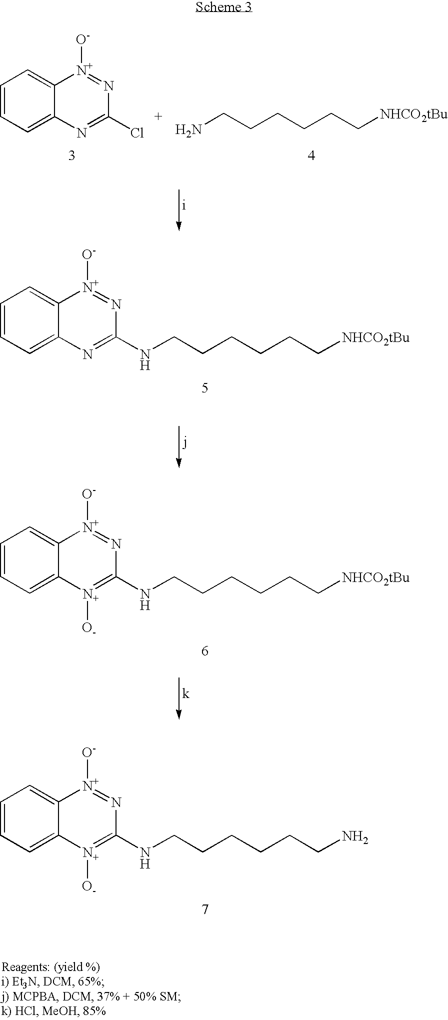

An alternative approach to using trifluoroacetic anhydride to provide protection for the primary amine and to generate trifluoroperacetic acid in situ was also used (Scheme 4). Deprotection of carbamate 5 gave the amine 8. Reaction of 8 with trifluoroacetic anhydride followed by 30% H₂O₂ gave a mixture of the 1-oxide 9 (22% yield) and 1,4-dioxide 10 (51% yield). 1-Oxide 9 was oxidised with trifluoroperacetic acid to give 10 (29% yield) as well as starting material 9 (61% yield). Deprotection of the trifluoroacetamide 10 provided 1,4-dioxide 7 in good yield.

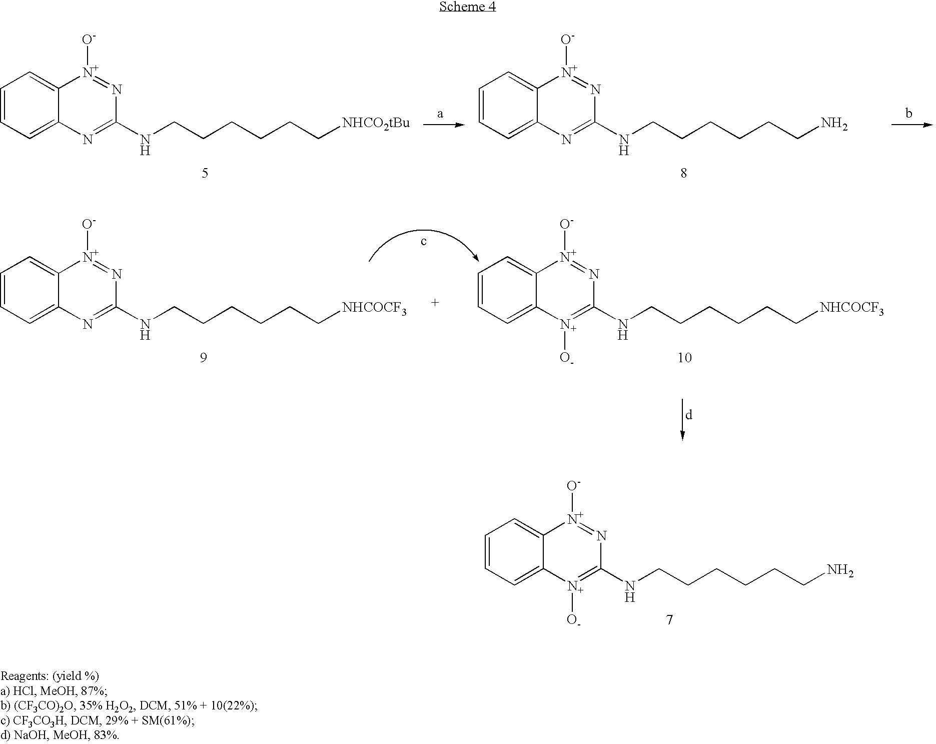

Coupling of 1,4 dioxide 7 with 9-methoxyacridine (Albert, “The Acridines” 2^{nd} ed. 1966, Edward Arnold, London, p. 281) provided a compound of Formula I: the aminoacridine derivative 11 (Scheme 5). Similarly, reaction of 7 with 4-(1H-imidazol-1-ylcarbonyl)acridine (Spicer et al., Anti-Cancer Drug Des., 1999, 14, 281-289) gave 12, a compound of Formula I. Similarly, reaction of the imidazolide of quinoline 4-acetic acid gave 13, a compound of Formula I.

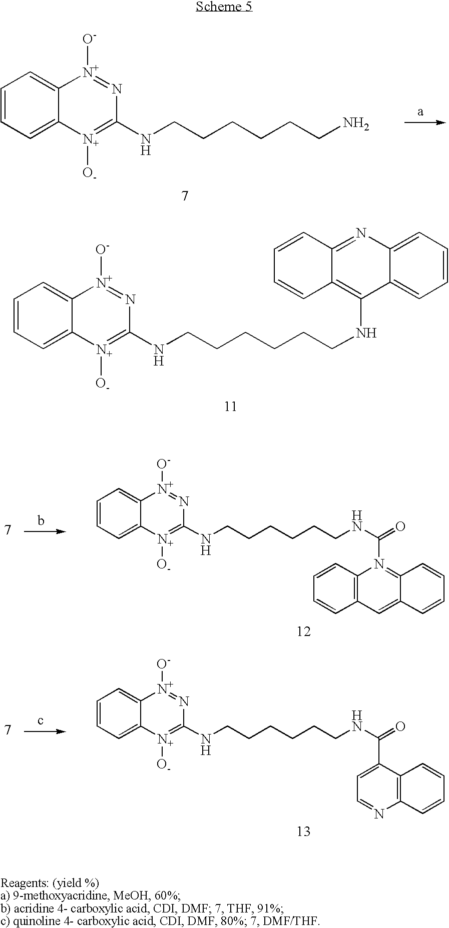

Reaction of chloride 3 with tert-butyl 3-aminopropylcarbamate gives 14, which was oxidised to 1,4-dioxide 15 with MCPBA (Scheme 6). Deprotection of 15 under acid conditions gave anine 16 which was reacted with 4-(1H-imidazol-1-ylcarbonyl)acridine to give 17, a compound of Formula I.

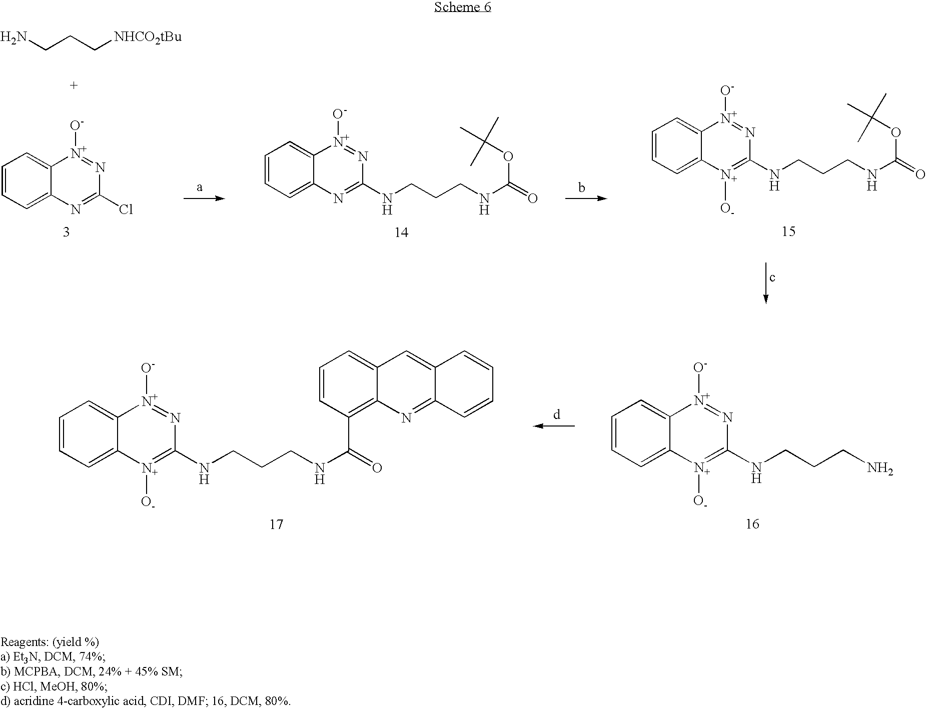

Coupling of chloride 3 with 2-(aminoethoxy)ethanol gave alcohol 18 in 63% yield which was converted to the mesylate and displaced with sodium azide to give azide 19 in 89% yield (Scheme 7). Selective reduction of the azide group rather than 1-oxide of 19 could not be effected by hydrogenation using palladium on charcoal or Lindlar catalyst (Rolla et al., J. Org. Chem., 1965, 47, 4322-432). Other methods for reducing azides such as NaBH₄ under PTC (Corey et al., Synth., 1975, 590-591), BH₃.DMS (Hassner & Levy, J. Amer. Chem. Soc., 1965, 87, 4203-4204) or Staudinger conditions using P(OEt)₃ (Koziara & Zwierzak, Synth., 1992, 1063-1065) were ineffective. However, treatment of azide 19 with propane-1,3-dithiol and Et₃N in refluxing methanol (Bayley et al., Tet. Lett., 1978, 39, 3633-3634) provided the intermediate amine which was protected without isolation with di-tert-butyldicarbonate to give carbamate 20 in 93% yield for the two steps. Oxidation of 20 with MCPBA gave 1,4-dioxide 21 in 40% yield as well as recovered staring material (50%). Deprotection of 21 with trifluoroacetic acid gave amine 22 in 91% yield. Coupling of 22 with 4-(1H-imidazol-1-ylcarbonyl)acridine gave compound 23 in 97% yield.

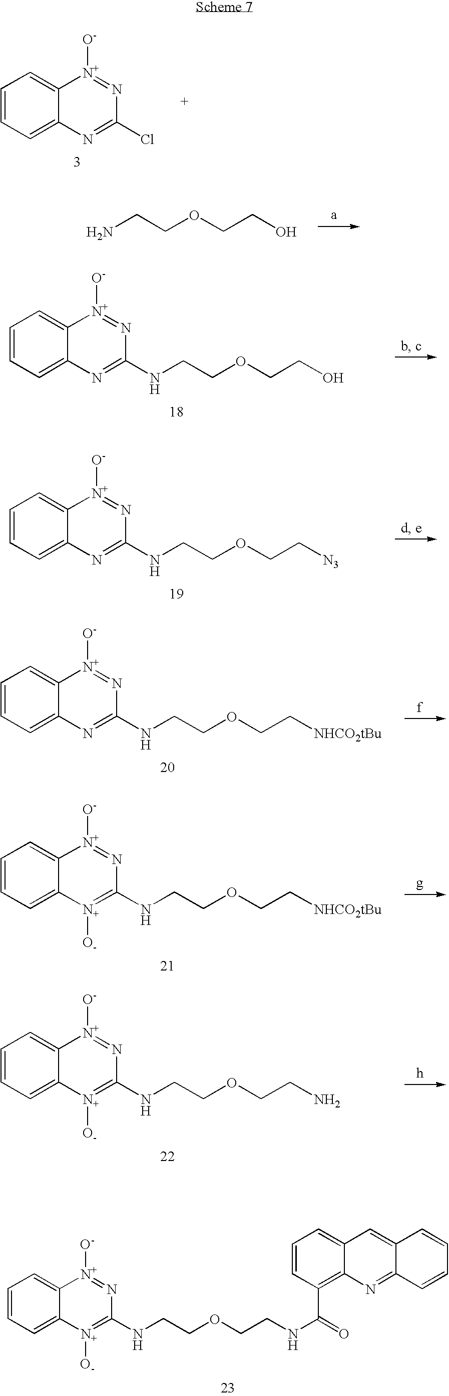

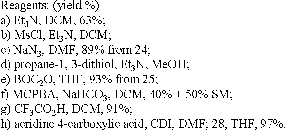

Similarly, reaction of 22 with the imidazolides of 8-quinolinecarboxylic acid, 2-phenyl-1H-benzimidazole-4-carboxylic acid (Denny et al., J. Med. Chem. 1990, 33, 814-819) and 2-(4-pyridinyl)-8-quinolinecarboxylic acid (Atwell et al., J. Med. Chem. 1989, 32, 396-401) gave compounds of Formula I: 24, 25, and 26 respectively (Scheme 8).

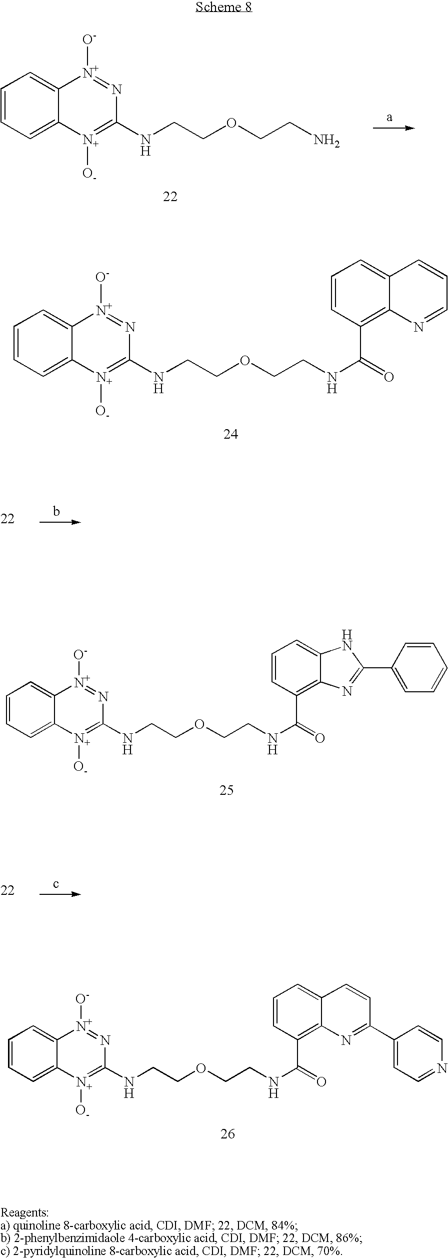

Reaction of chloride 3 with tert-butyl bis(3-aminopropyl)carbamate and protection of the intermediate primary amine with trifluoroacetic anhydride gave the trifluoroacetamide 27 in 39% for the two steps (Scheme 9). Oxidation of 27 with MCPBA gave the 1,4-dioxide 28 (8% with 65% recovered starting material). Deprotection of 28 gave amine 29 in good yield which was coupled to 4-(1H-imidazol-1-ylcarbonyl)acridine to give compound 30, a compound of Formula I.

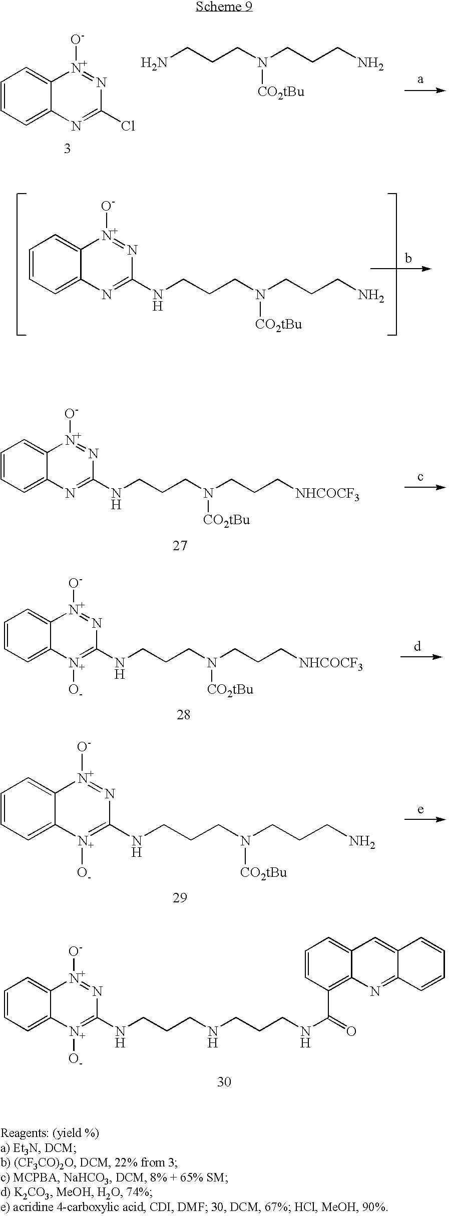

Similarly, reaction of amine 29 with imidazolides of phenazine and 9-methylphenazine followed by deprotection under acidic conditions gave compounds 31 and 32, respectively (Scheme 10).

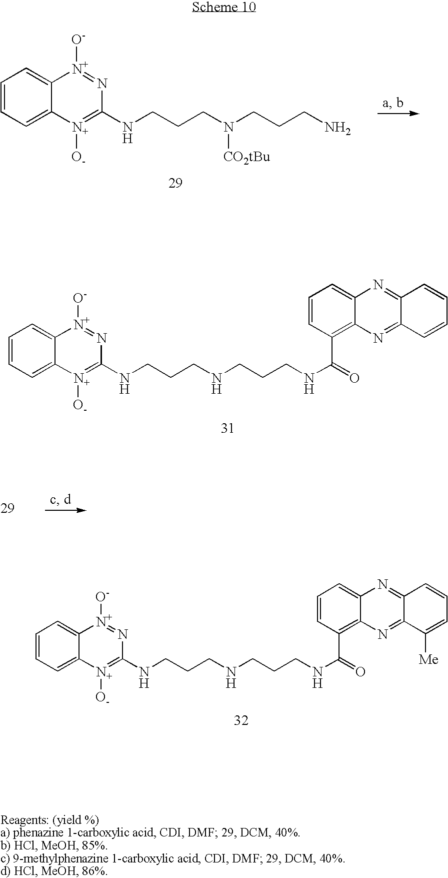

Reaction of chloride 3 with amine 33, prepared from N¹-(2-aminoethyl)-1,2-ethanediamine gave the 1-oxide 34 (Scheme 11). Compound 34 was protected as carbamate 35 and oxidized with MCPBA to give dioxide 36. Deprotection and coupling of the intermediate amine with the imidazolide of acridine 4-carboxylic acid gave compound 37, a compound of Formula I.

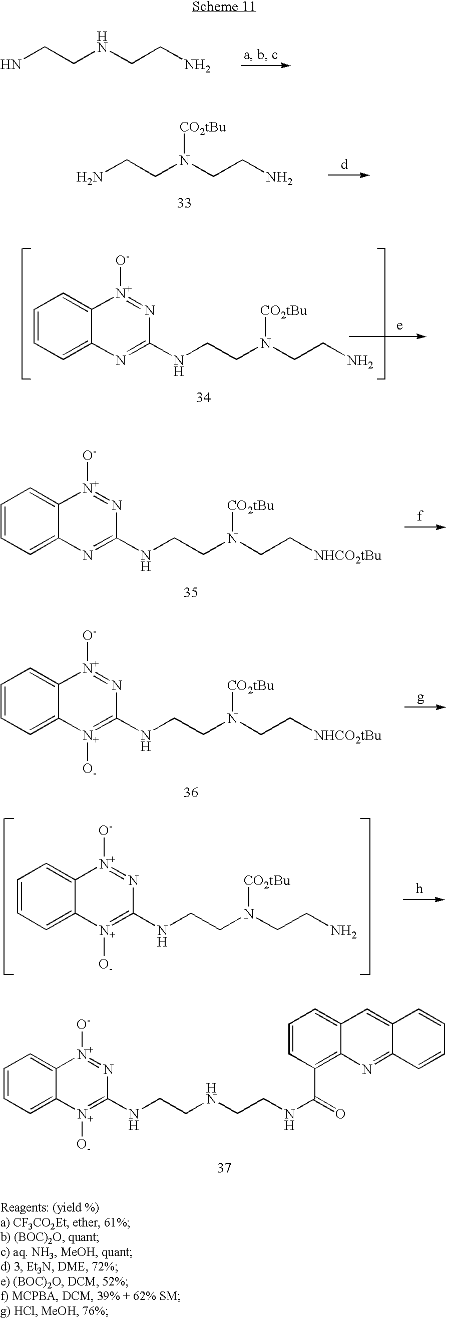

Reaction of chloride 3 with N¹-(3-aminopropyl)-N¹-methyl-1,3-propanediamine and protection of the intermediate amine gave acetamide 318 in 43% yield (Scheme 12). Oxidation of 38 with trifluoroperacetic acid under acidic conditions resulted in selective aromatic N-oxidation to give 1,4-dioxide 39 (27%) and recovered standing material 38 (24%). Deprotection of 39 gave amine 40 which was coupled with 4-(1H-imidazol-1-ylcarbonyl)acridine to give compound 41, a compound of Formula I, in 66% yield.

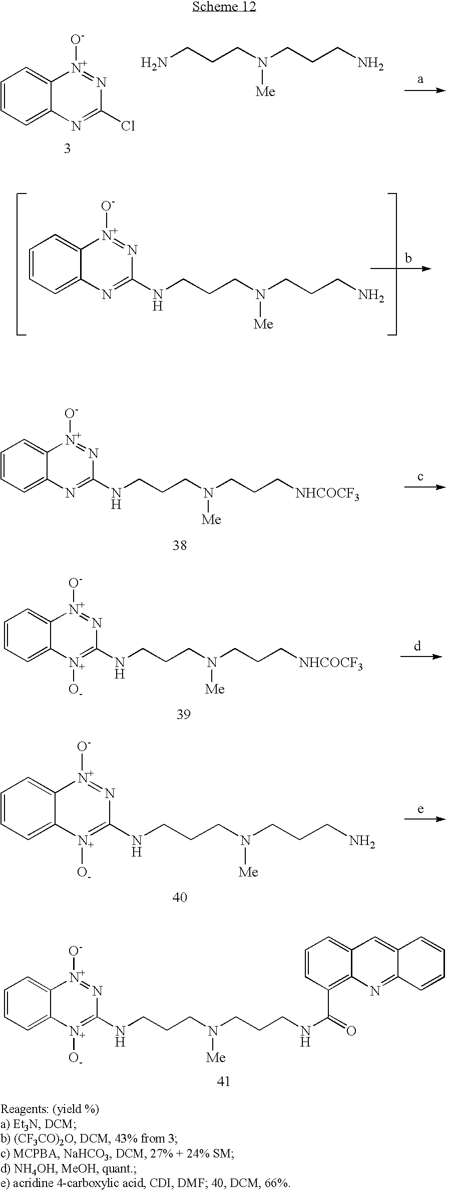

Similarly, reaction of 40 with the imidazolides of 8-quinolinecarboxylic acid, 2-(4-pyridinyl)-S-quinolinecarboxylic acid (Atwell et al., J. Med. Chem. 1989, 32, 396-401), 5-methyl-4-acridine carboxylic acid, and 9-methyl-4-phenazinecarboxylic acid gave compounds 42, 43, 44, and 45 respectively (Scheme 13).

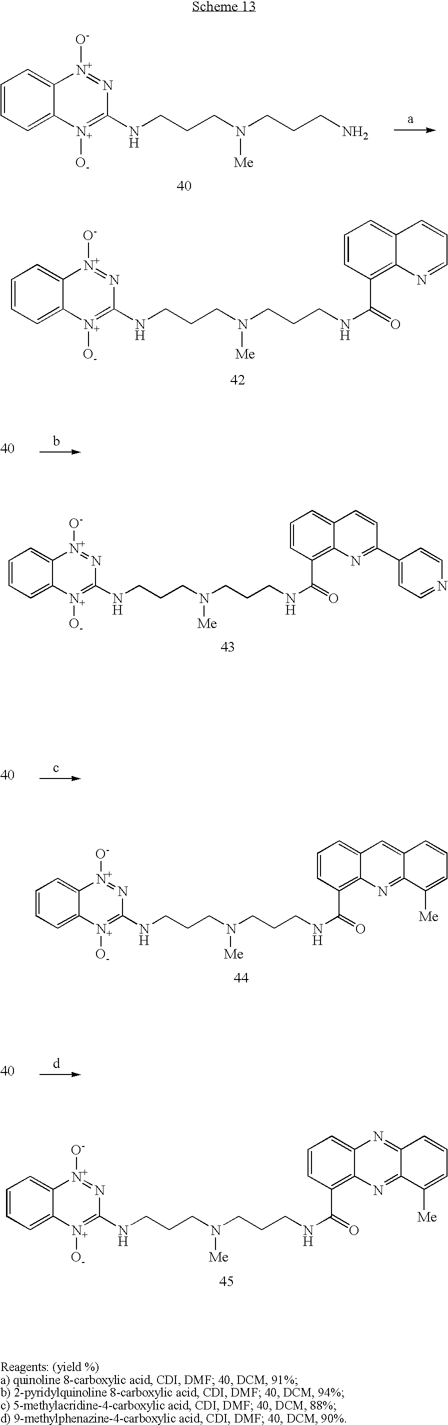

Reaction of 4-amino-3-nitrophenol with cyanamide under acidic conditions followed by condensation under basic conditions gave the phenol 46 (Friebe et. al. U.S. Pat. No. 5,856,325, Jan. 5, 1999), which was alkylated under basic conditions to give ether 47 (Scheme 14). Diazotization of 47 gave 48, which was chlorinated with POCl₃ to give chloride 49. Coupling of chloride 49 with amine 50 gave the 1-oxide 51. Protection of 51 as the trifluoroacetamide 52 and oxidation with trifluoroperacetic acid gave the dioxide 53. Deprotection of 53 gave intermediate amine 54, which was coupled with the imidazolide of acridine-4-carboxylic acid to give compound 55, a compound of Formula I.

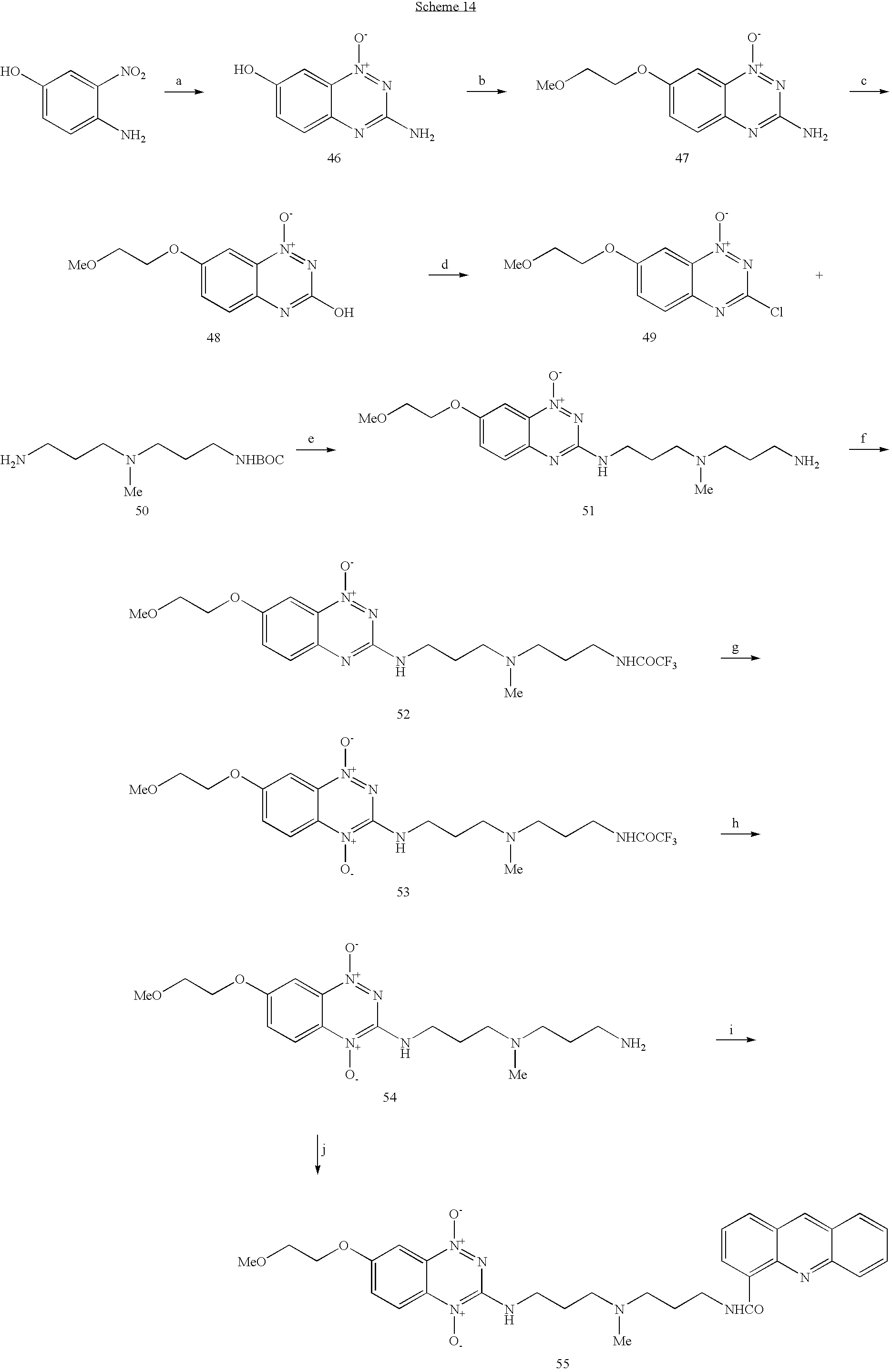

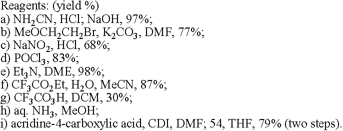

Reaction of chloride 3 with allyltributyltin in the presence of tetrakis-palladiumtriphenylphosphine in DME at reflux temperature gave alkene 56 in high yield (Scheme 15). Hydroboration of 56 gave the alcohol 57 which was activated with methanesulfonyl chloride and reacted with tert-butyl 3-aminopropylcarbamate to give the amine 58. Conversion to the trifluoroacetamide 59 and oxidation with trifluoroperacetic acid gave the 1,4-dioxide 60, which was deprotected under basic conditions to give amine 61. Coupling of amine 61 with the imidazolide of acridine-4-carboxylic acid gave compound 62, a compound of Formula I. Similarly, coupling of amine 61 with the imidazolide of phenazine-4-carboxylic acid gave compound 63, a compound of Formula I. 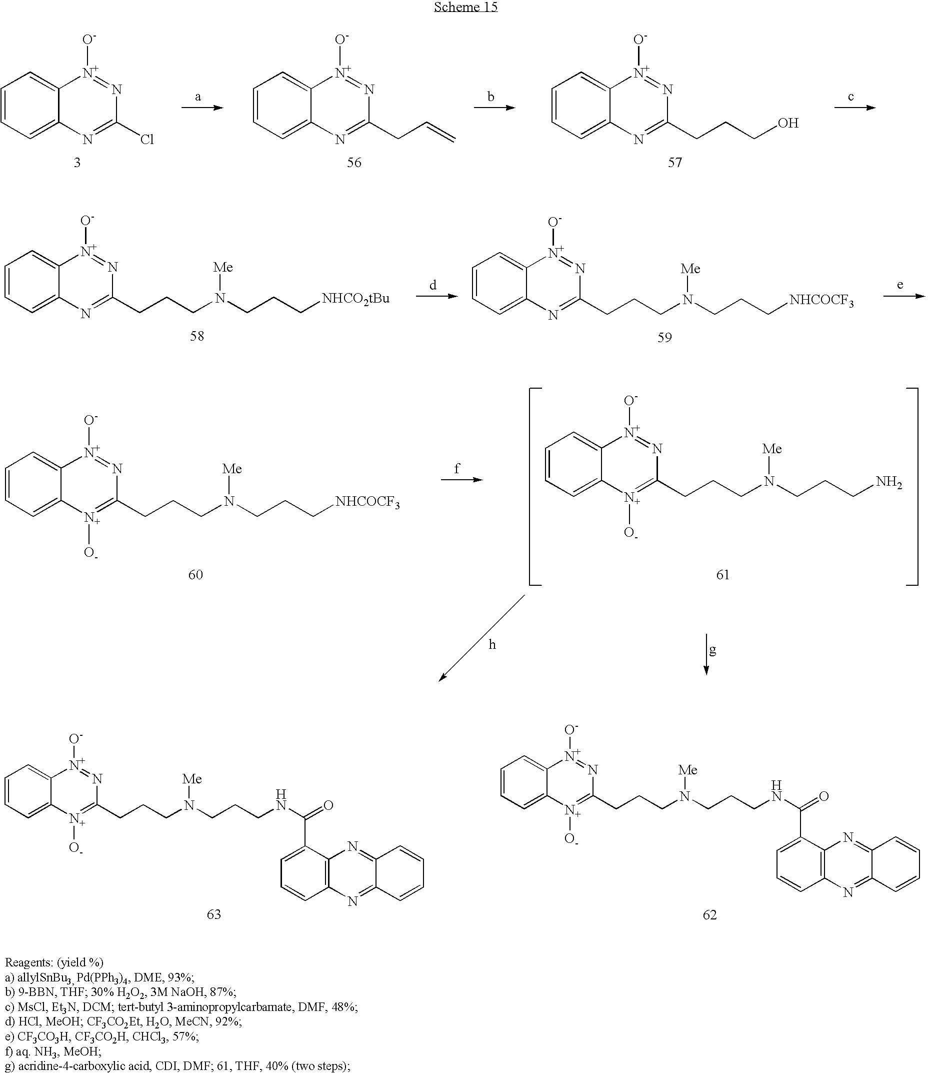
Reaction of amine 16 with the imidazolide of N-(7-chloro-4-quinolinyl)-β-alamine (64) (Titus et al, J. Org. Chem., 1948, 13, 39-62) gave amide 65, a compound of Formula I (Scheme 16).

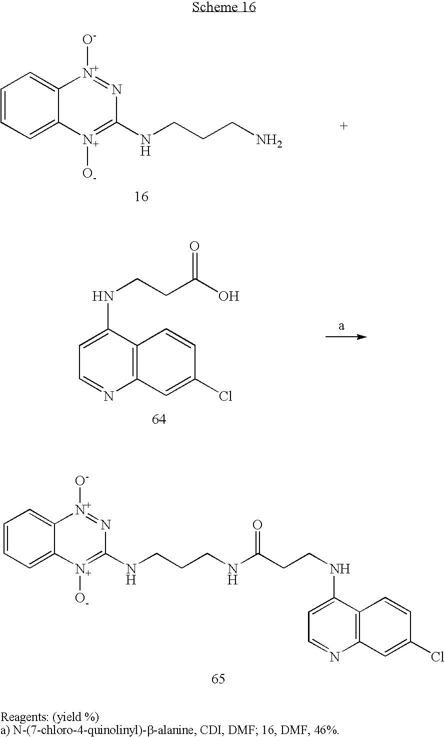

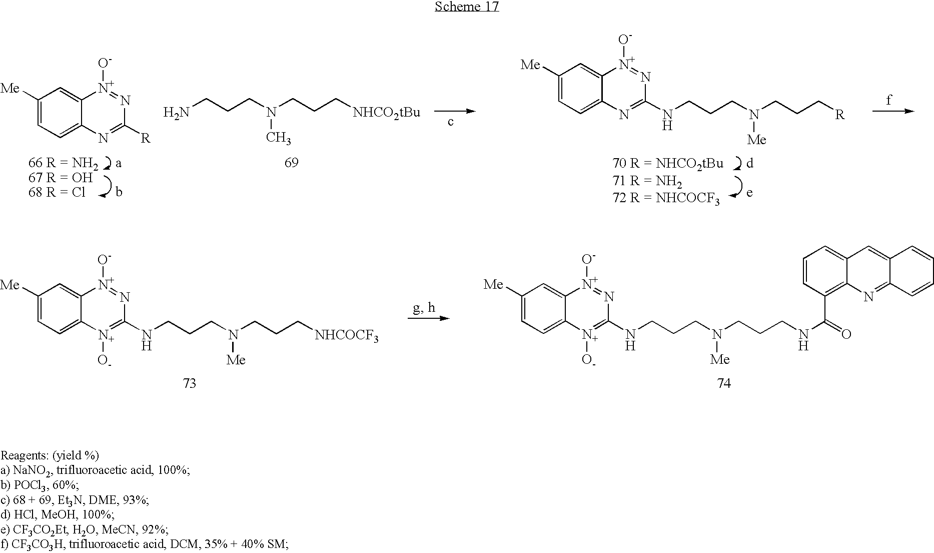

Similarly, deprotection of 73 and reaction with the imidazolides of 2-(4-pyridinyl)-8-quinolinecarboxylic acid (Atwell et al., J. Med. Chem. 1989, 32, 396-401), phenazine-1-carboxylic acid (Rewcastle et al., J. Med. Chem. 1987, 30, 843-851) and 9-methylphenazine-1-carboxylic acid (Rewcastle et al., J. Med. Chem. 1987, 30, 843-851) gave compounds of Formula I: 75, 76, and 77 respectively (Scheme 18).

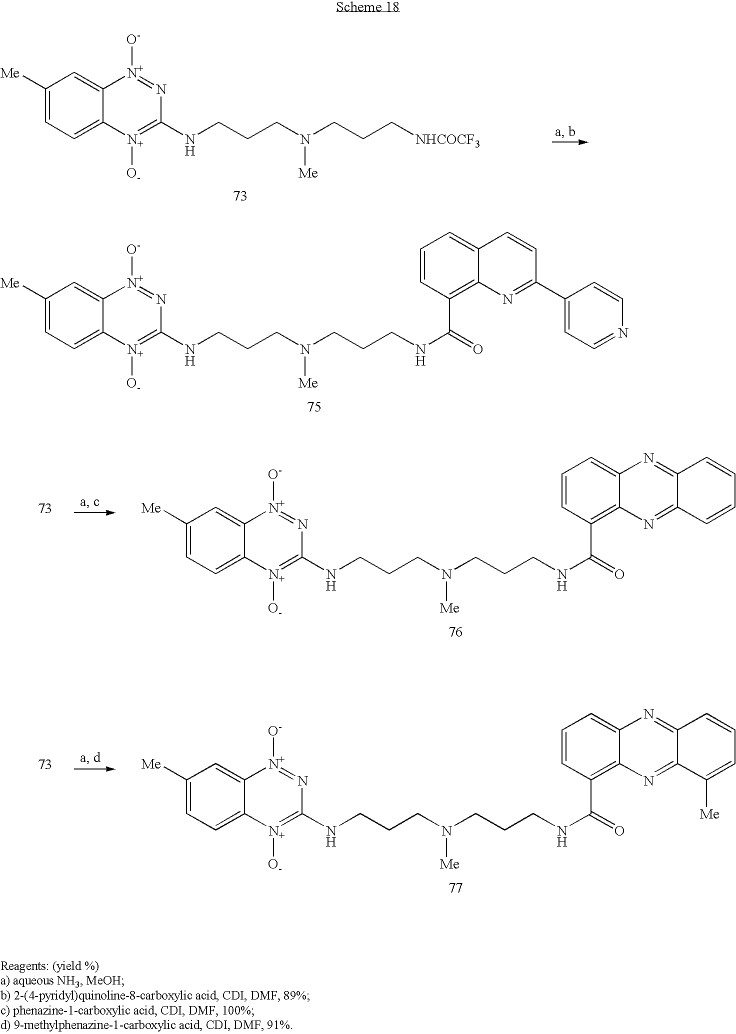

Diazotization of amine 78 [Hay et al, J. Med. Chem. 2003, 46, 169-182] with sodium nitrite in trifluoroacetic acid gave the alcohol 79 (Scheme 19) which was converted to chloride 80 in POCl₃. Coupling of chloride 80 with the mono-protected amine 69 gave carbamate 81 which was deprotected under acidic conditions to give amine 82 which was reprotected as the trifluoroacetate 83. Oxidation of 83 with trifluoroperacetic acid gave 1,4-dioxide 84 which was deprotected under basic conditions and coupled to the imidazolide of acridine-4-carboxylic acid (Spicer et al., Anti-Cancer Drug Des., 1999, 14, 281-289) to give compound 85.

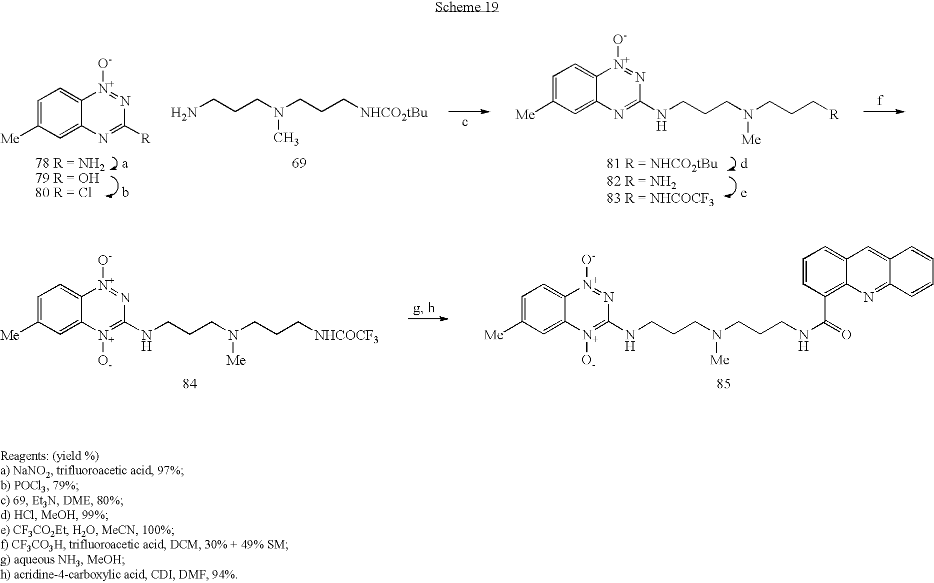

Similarly, deprotection of 84 and reaction with the imidazolides of 2-(4-pyridinyl)-8-quinolinecarboxylic acid, phenazine-1-carboxylic acid and 9-methylphenazine-1-carboxylic acid gave compounds of Formula I: 86, 87, and 88 respectively (Scheme 20).

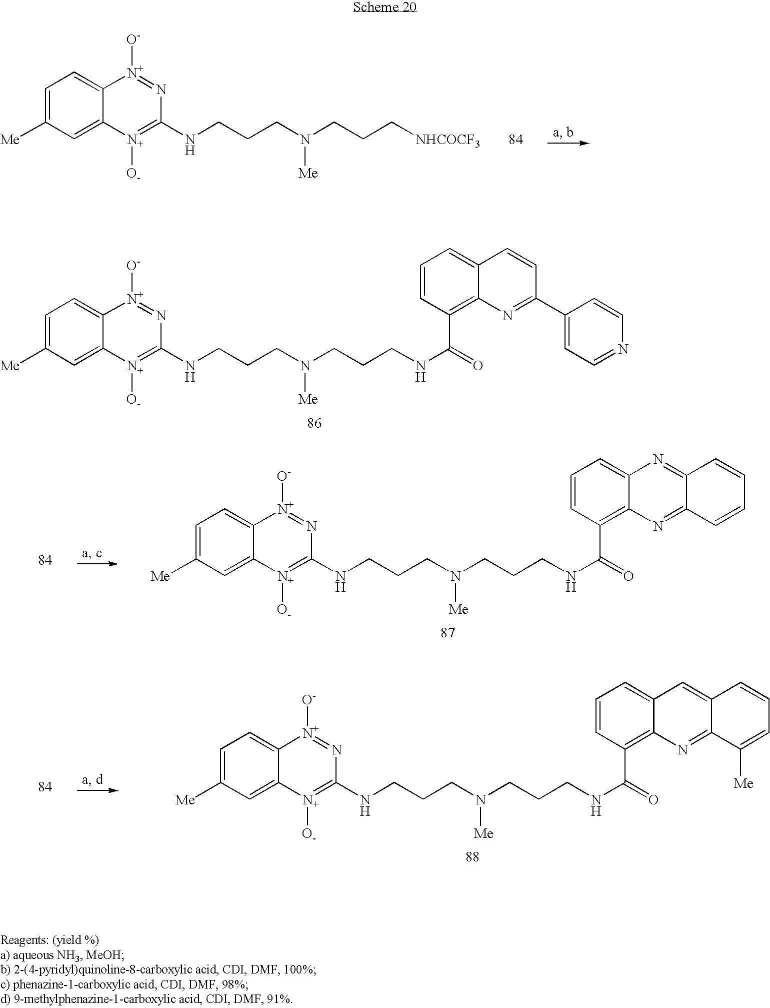

Coupling of chloride 3 with the mono-protected amine 90, prepared from 89, gave carbamate 91 which was deprotected under acidic conditions to give amine 92 which was reprotected as the trifluoroacetate 93 (Scheme 21). Oxidation of 9-3 with trifluoroperacetic acid save 1,4-dioxide 94 which was deprotected under basic conditions and coupled to the imidazolide of acridine-4-carboxylic acid to give compound 95.

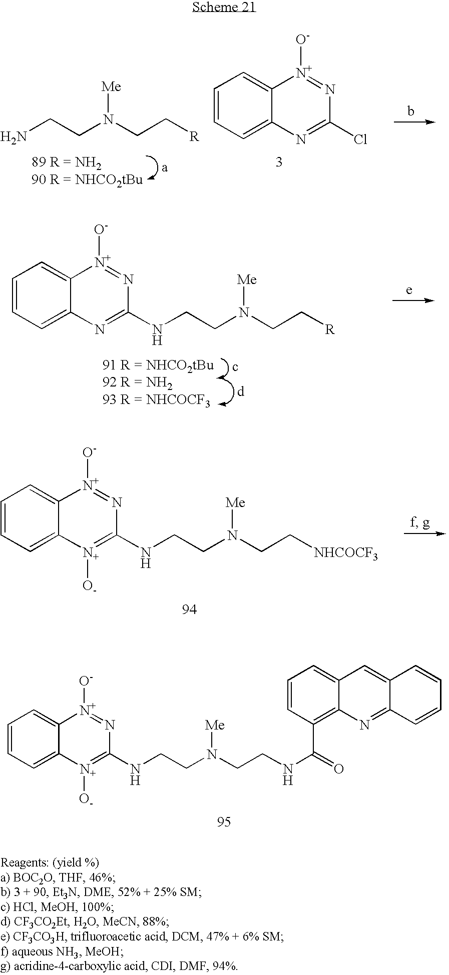

Similarly, deprotection of 94 and reaction with the imidazolides of 2-(4-pyridinyl)-8-quinolinecarboxylic acid, phenazine-1-carboxylic acid, 9-methylphenazine-1-carboxylic acid and 5-methylacridine-4-carboxylic acid gave compounds 96, 97, 98, and 99 respectively (Scheme 22).

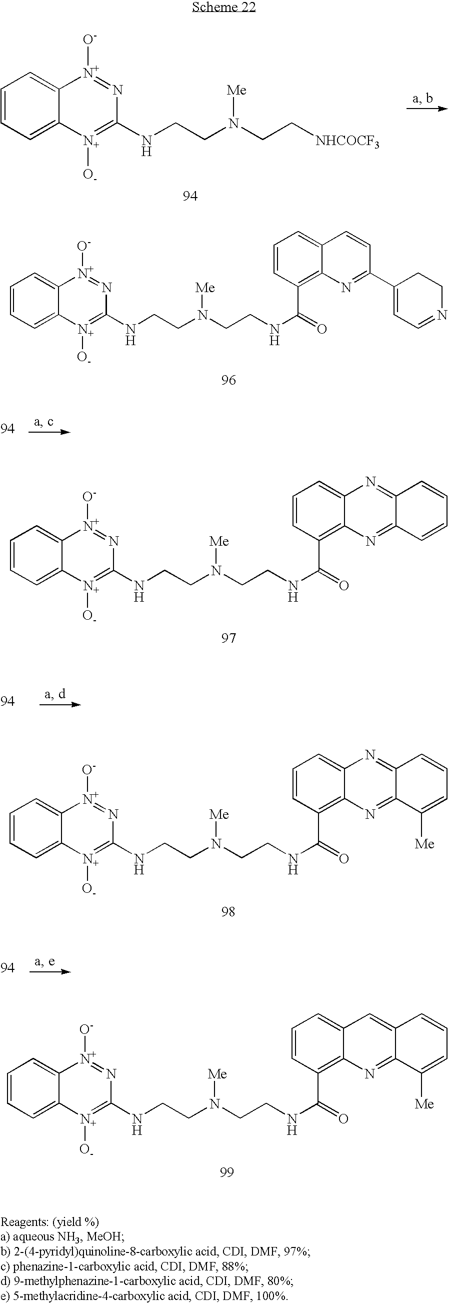

Deprotection of trifluoroacetamide 39 under basic conditions and reaction with the imidazolide of phenazine-1-carboxylic acid gave compounds 100 (Scheme 23).

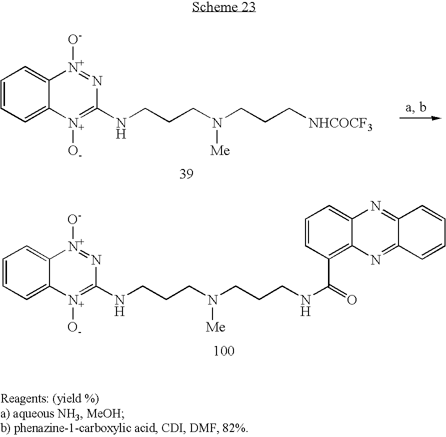

Deprotection of 36 and reaction with the imidazolides of 2-(4-pyridinyl)-8-quinolinecarboxylic acid and 5-methylacridine-4-carboxylic acid gave compounds 102 and 103 respectively (Scheme 24).

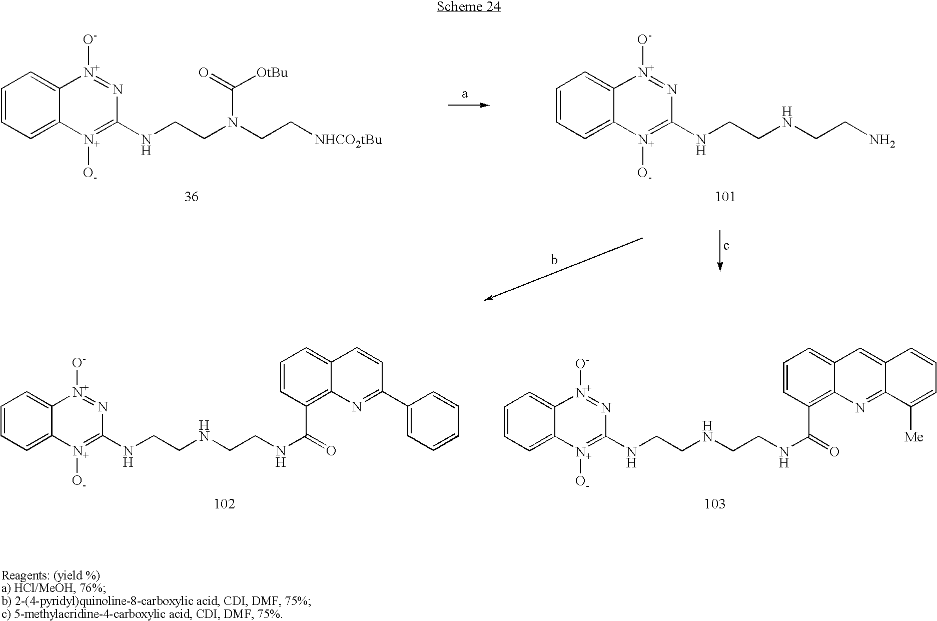

Coupling of acid 104 (Baird & Dervan, J. Am. Chem. Soc. 1996, 118, 6141-6146) and amine 105 (Baird & Dervan, J. Am. Chem. Soc. 1996, 118, 6141-6146) with EDCI and DMAP gave ester 106 (Scheme 25) which was hydrolysed under basic conditions to give acid 107. Coupling of acid 107 and 3-dimethylaminopropylamine with EDCI and DMAP gave amide 108. Reaction of chloride 3 with methyl 4-aminobutanoate gave ester 109 which was oxidised with trifluoroperacetic acid to give 1,4-dioxide 110 which was hydrolysed to acid 111. Deprotection of carbamate 108 followed by coupling to acid 111 with EDCI and DMAP gave compound 112.

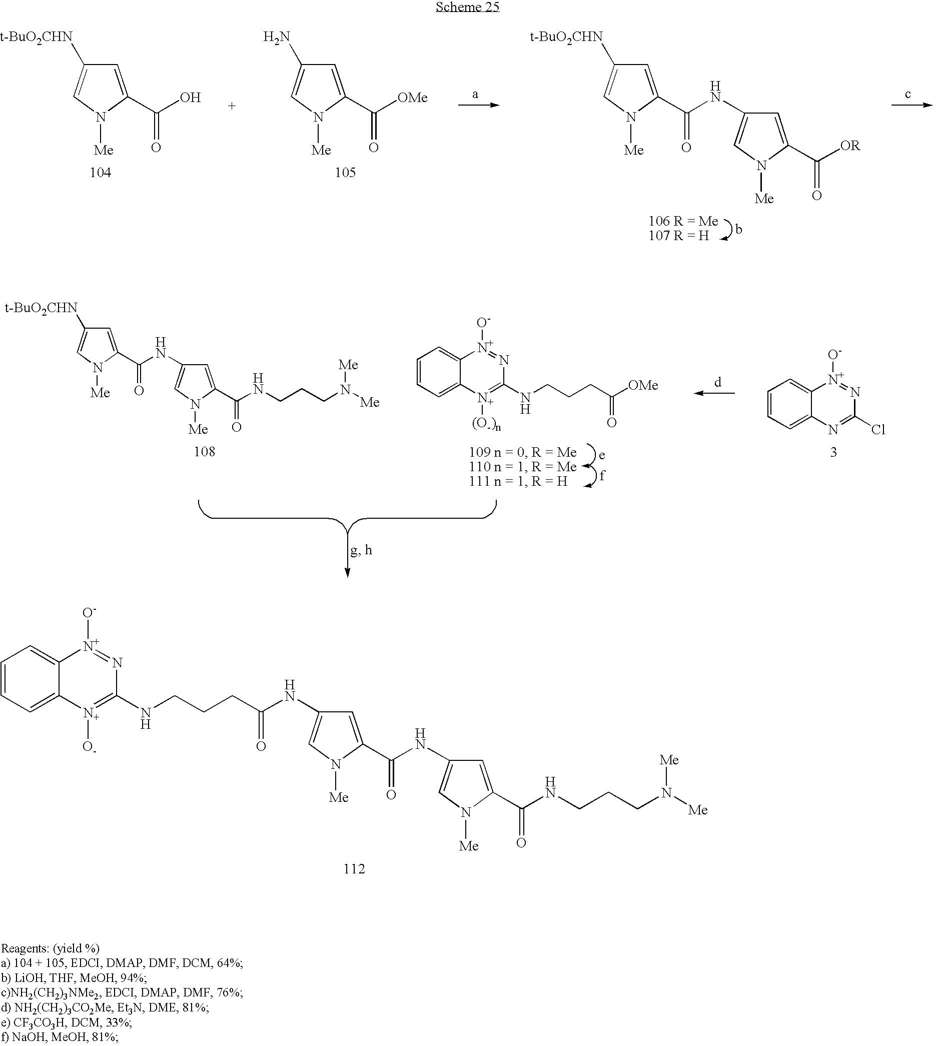

Reaction of the phenol 46 with the protected bromide gave compound 113 (Scheme 26) which underwent diazotization to the 3-hydroxy intermediate and chlorination with POCl₃ to give chloride 114. Stille coupling with tetraethyltin in the presence of a palladium catalyst gave the 3-ethyl compound 115. Deprotection followed by reprotection with dibutyldicarbonate gave compound 116 which was oxidised with MCPBA to give dioxide 116. Deprotection and coupling of the imidazolide of acridine-4-carboxylic acid is expected to afford compound 118, a compound of Formula I′.

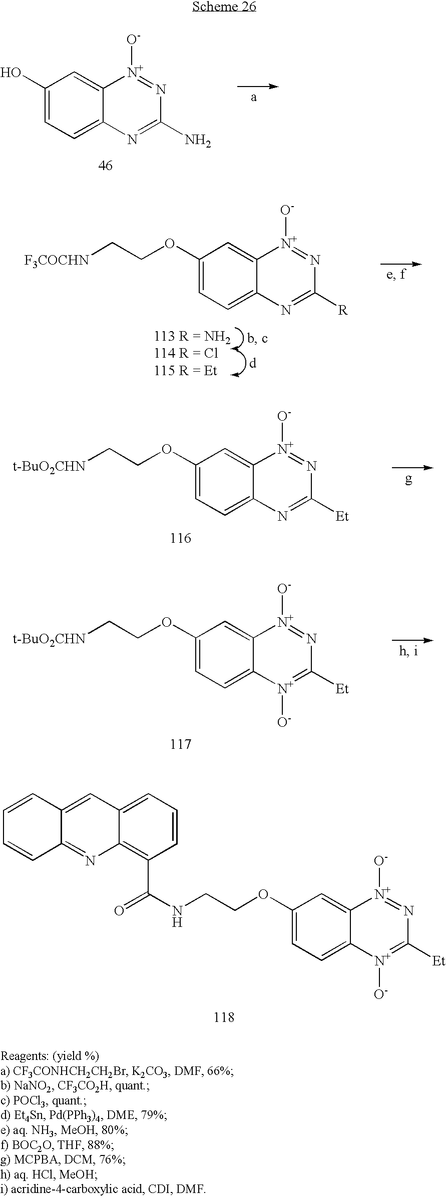

### EXAMPLES OF THE COMPOUNDS OF THE INVENTION

Table 1 gives details on examples of compounds within the scope of the invention, and preparable by methods of the invention.

TABLE 1
Table 1. Examples of compounds

| | |
|---|---|
| | I |
| 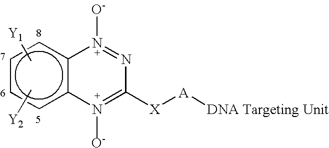 | |

| No. | # | Y₁ | Y₂ | X | A | DNA targeting unit | mp (° C.) | Anal |
|---|---|---|---|---|---|---|---|---|
| 11 | 3 | H | H | NH | (CH₂)₆ | 9-NHacridine | 118-119 | C, H, N |
| 12 | 3 | H | H | NH | (CH₂)₆ | 4-NHCOacridine | 196-198 | C, H, N |
| 13 | 3 | H | H | NH | (CH₂)₆ | 4-NHquinoline | 196-198 | C, H, N |
| 17 | 3 | H | H | NH | (CH₂)₃ | 4-NHCOacridine | 192 | C, H, N |
| 23 | 3 | H | H | NH | (CH₂)₂O(CH₂)₂ | 4-NHCOacridine | 98-100 | C, H, N |
| 24 | 3 | H | H | NH | (CH₂)₂O(CH₂)₂ | 8-NHCOquinoline | 168-170 | C, H, N |
| 25 | 3 | H | H | NH | (CH₂)₂O(CH₂)₂ | 4-NHCObenz-imidazole-2-phenyl | 203-207 | C, H, N |
| 26 | 3 | H | H | NH | (CH₂)₂O(CH₂)₂ | 8-NHCOquinoline-2-(4-pyridyl) | 128-130 | C, H, N |
| 30 | 3 | H | H | NH | (CH₂)₃NH(CH₂)₃ | 4-NHCOacridine | gum | C, H, N |
| 31 | 3 | H | H | NH | (CH₂)₃NH(CH₂)₃ | 1-NHCOphenazine | 163-169 | C, H, N |
| 32 | 3 | H | H | NH | (CH₂)₃NH(CH₂)₃ | 1-NHCO-9-methyl-phenazine | 183-186 | HRMS |
| 37 | 3 | H | H | NH | (CH₂)₂NH(CH₂)₂ | 4-NHCOacridine | 151-154 | C, H, N |
| 41 | 3 | H | H | NH | (CH₂)₃NMe(CH₂)₃ | 4-NHCOacridine | 169-171 | HRMS |
| 42 | 3 | H | H | NH | (CH₂)₃NMe(CH₂)₃ | 8-NHCOquinoline | 119-121 | C, H, N |
| 43 | 3 | H | H | NH | (CH₂)₃NMe(CH₂)₃ | 8-NHCO-2-(4-pyridyl)quinoline | 179-181 | C, H, N |
| 44 | 3 | H | H | NH | (CH₂)₃NMe(CH₂)₃ | 4-NHCO-5-methylacridine | 158-162 | C, H, N |
| 45 | 3 | H | H | NH | (CH₂)₃NMe(CH₂)₃ | 1-NHCO-9-methylphenazine | 138-142 | C, H, N |
| 55 | 3 | † | H | NH | (CH₂)₃NMe(CH₂)₃ | 4-NHCOacridine | 98-103 | HRMS |
| 62 | 3 | H | H | CH₂ | (CH₂)₂NMe(CH₂)₃ | 4-NHCOacridine | gum | HRMS |
| 63 | 3 | H | H | CH₂ | (CH₂)₂NMe(CH₂)₃ | 1-NHCOphenazine | 173 | HRMS |
| 65 | 3 | H | H | NH | (CH₂)₃NHCO(CH₂)₂ | 4-NH-7-Clquinoline | 202 | HRMS |
| 74 | 3 | 7-Me | H | NH | (CH₂)₃NMe(CH₂)₃ | 4-NHCOacridine | 166-168 | C, H, N |
| 75 | 3 | 7-Me | H | NH | (CH₂)₃NMe(CH₂)₃ | 8-NHCO-2-(4-pyridyl)quinoline | 178-180 | C, H, N |
| 76 | 3 | 7-Me | H | NH | (CH₂)₃NMe(CH₂)₃ | 1-NHCOphenazine | 118-122 | C, H, N |
| 77 | 3 | 7-Me | H | NH | (CH₂)₃NMe(CH₂)₃ | 1-NHCO-9-methyl-phenazine | 119-122 | C, H, N |
| 85 | 3 | 6-Me | H | NH | (CH₂)₃NMe(CH₂)₃ | 4-NHCOacridine | 158-160 | C, H, N |
| 86 | 3 | 6-Me | H | NH | (CH₂)₃NMe(CH₂)₃ | 8-NHCO-2-(4-pyridyl)quinoline | 178-180 | C, H, N |
| 87 | 3 | 6-Me | H | NH | (CH₂)₃NMe(CH₂)₃ | 1-NHCOphenazine | 111-114 | C, H, N |
| 88 | 3 | 6-Me | H | NH | (CH₂)₃NMe(CH₂)₃ | 1-NHCO-9-methyl-phenazine | 80-83 | HRMS |
| 95 | 3 | H | H | NH | (CH₂)₂NMe(CH₂)₂ | 4-NHCOacridine | 160-162 | HRMS |
| 96 | 3 | H | H | NH | (CH₂)₂NMe(CH₂)₂ | 8-NHCO-2-(4-pyridyl)quinoline | 130-135 | C, H, N |
| 97 | 3 | H | H | NH | (CH₂)₂NMe(CH₂)₂ | 1-NHCOphenazine | 163-165 | C, H, N |
| 98 | 3 | H | H | NH | (CH₂)₂NMe(CH₂)₂ | 1-NHCO-9-methyl-phenazine | 161-163 | C, H, N |
| 99 | 3 | H | H | NH | (CH₂)₂NMe(CH₂)₂ | 4-NHCO-5-methylacridine | 148-152 | C, H, N |
| 100 | 3 | H | H | NH | (CH₂)₃NMe(CH₂)₃ | 1-NHCOphenazine | 129-130 | C, H, N |
| 102 | 3 | H | H | NH | (CH₂)₂NH(CH₂)₂ | 8-NHCO-2-(4-pyridyl)quinoline | 160-165 | C, H, N |
| 103 | 3 | H | H | NH | (CH₂)₂NH(CH₂)₂ | 4-NHCO-5-methylacridine | 135-140 | HRMS |
| 112 | 3 | H | H | NH | (CH₂)₃CONH | 3-pyr-5-CONH-3-pyr-5-CONH(CH₂)₃NMe₂ | 140-145 | C, H, N |

# Side chain position
†7-MeOCH₂CH₂O—

In the following examples representative of the invention and the detailed methods for preparing them:

Elemental analyses were carried out in the Microchemical Laboratory, University of Otago, Dunedin, NZ.

Melting points were determined on an Electrothermal 2300 Melting Point Apparatus. IR spectra were recorded on a Midac FT-IR as KBr discs, unless otherwise stated.

NMR spectra were obtained on a Bruker Avance-400 spectrometer at 400 MHz for ¹H and 100 MHz for ¹³C spectra. Spectra were obtained in CDCl₃ unless otherwise specified, and are referenced to Me₄Si. Chemical shifts and coupling constants were recorded in units of ppm and Hz, respectively. Assignments were determined using COSY, HSQC, and HMBC two-dimensional experiments.

Mass spectra were determined on a VG-70SE mass spectrometer using an ionizing potential of 70 eV at a nominal resolution of 1000. High-resolution spectra were obtained at nominal resolutions of 3000, 5000, or 10000 as appropriate. All spectra were obtained as electron impact (EI) using PFK as the reference unless otherwise stated. Solutions in organic solvents were dried with anhydrous Na₂SO₄. Solvents were evaporated under reduced pressure on a rotary evaporator.

Thin-layer chromatography was carried out on aluminium-backed silica gel plates (Merck 60 F₂₅₄) with visualization of components by UV light (254 nm) or exposure to I₂.

Column chromatography was carried out on silica gel, (Merck 230-400 mesh). All compounds designated for testing were analyzed at >99% purity by reverse phase HPLC using an Agilent 1100 liquid chromatograph, an Alltima C₁₈ (5μ) stainless steel column (150 mm×3.2 mm i.d.) and an Agilent 1100 diode array detector. Chromatograms were run using various gradients of aqueous (0.045 M ammonium formate and formic acid at pH 3.5) and organic (80% MeCN/MilliQ water) phases. DCM refers to dichloromethane; ether refers to dimethoxyethane, DMF refers to dry dimethyl formamide; ether refers to diethyl ether; EtOAc refers to ethyl acetate; EtOH refers to ethanol; MeOH refers to methanol; pet. ether refers to petroleum ether, boiling range 40-60° C.; THF refers to tetrahydrofuran dried over sodium benzophenone ketyl. All solvents were freshly distilled.

### Example A

### 3-[(6-Aminohexyl)amino]-1,2,4-benzotriazine 1,4-dioxide (7)

3-Chloro-1,2,4-benzotriazine 1-oxide (3). 2-Nitroaniline (10 g, 72.4 mmol) and cyanamide (14.0 g, 330 mmol) were melted together and cHCl (20 mL) added cautiously. The mixture was heated at 100° C. until the foaming subsided. The mixture was made strongly alkaline with 30% w/v NaOH and heated at 100° C. for 10 min. The suspension was cooled to 25° C. and the yellow solid filtered, washed with water (20 mL) and dried. A small sample was recrystallized to give 3-amino-1,2,4-benzotriazine 1-oxide (1) mp (MeOH/EtOAc) 267-269° C.; lit. [Arndt, Ber. 1913, 46, 3522-3529] mp (EtOH) 269° C.]. The remainder was dissolved in 2 M HCl (300 mL), cooled to 5° C., and a solution of NaNO₂ (10 g, 0.145 mol) in water (100 mL) added dropwise. The resulting precipitate was filtered, dissolved in dilute NH₃, filtered, and acidified with cHCl. The precipitate was filtered, washed with water and dried to give 3-hydroxy-1,2,4-benzotriazine 1-oxide (2) (5.77 g, 49%) as a yellow powder, mp 209-212° C.; lit. [Arndt, Ber. 1913, 46, 3522-3529] mp (H₂O) 219° C.]; ¹H NMR [(CD₃)₂SO] δ 8.14 (d, J=8.4 Hz, 1H, H-8), 7.77-7.81 (m, 1H, H-6), 7.54 (d, J=8.4 Hz, 1H, H-5), 7.88-7.92 (m, 3H, H-7, NH₂); ¹³C NMR [(CD₃)₂SO] δ 160.2, 148.7, 135.6, 129.8, 125.8, 124.6, 119.8. A mixture of the alcohol (2) (5.7 g, 34.9 mmol), N,N-dimethylaniline (11 mL, 87.3 mmol), and POCl₃ (23 mL, 244 mmol) was heated at reflux temperature for 1 h then poured on to ice. The resulting solid was filtered and recrystallized to give 3-chloro-1,2,4-benzotriazine 1-oxide (3) (3.77 g, 59%) as a pale yellow powder. mp 119-119.5° C.; lit. [Robbins et al., J. Chem. Soc., 1957, 3186-3194] (MeOH) 117-118° C.]; ¹H NMR [(CD₃)₂SO] δ 8.38 (dd, J=8.7, 1.0 Hz, 1H, H-8), 8.16 (ddd, J=8.3, 7.0, 1.3 Hz, 1H, H-6), 8.06 (dd, J=8.2, 1.0 Hz, 1H, H-5), 7.90 (ddd, J=8.7, 6.9, 1.3 Hz, 1H, H-7); ¹³C NMR [(CD₃)₂SO] δ 15.3, 146.9, 137.2, 133.9, 131.5, 128.0, 119.9.

6-t-Butyloxycarbamoylhexylamine (4). A solution of di-t-butyldicarbonate (18.6 g, 85.3 mmol) in dry DCM (100 mL) was added dropwise to a stirred solution of 6-aminohexanol (10.0 g, 85.3 mmol) in dr DCM (100 mL) at 20° C. and stirred for 16 h. The solution was washed with dilute aqueous Na₂CO₃ solution (100 mL), 0.1 M HCl (100 mL), water (100 mL), brine (50 mL), dried and the solvent evaporated. The residue was dissolved in DCM (250 mL) and Et₃N (15.5 mL, 111 mmol) added. A solution of methanesulfonyl chloride (7.3 mL, 94 mmol) was added dropwise and the mixture stirred at 20° C. for 16 h. The solution was washed with saturated aqueous KHCO₃ (100 mL), water (2×100 mL), brine (50 mL), dried, and the solvent evaporated. The residue was dissolved in DMF (100 mL) and NaN₃ (5.55 g, 85.3 mmol) added. The mixture was stirred at 100° C. for 1 h, the solvent evaporated and the residue partitioned between EtOAc (200 mL) and water (200 mL). The organic fraction was washed with water (200 mL), brine (100 mL), dried and the solvent evaporated. The residue was purified by chromatography, eluting with 30% EtOAc/pet. ether, to give the 6-t-butyloxycarbamoylhexyl azide (17.5 g, 85%) as a colorless oil, ¹H NMR δ 4.53 (br s, 1H, OCONH), 3.52 (t, J=6.9 Hz, 2H, CH₂N), 3.11 (dt, J=6.5, 6.4 Hz, 2H, CH₂N), 1.57-1.63 (m, 2H, CH₂), 1.44-1.52 (m, 11H, CH₂, C(CH₃)₃), 1.30-1.40 (m, 4H, 2×CH₂); ¹³C NMR δ 156.0, 79.1, 51.3, 40.4, 29.9, 28.7, 28.4 (3), 126.4, 26.3. A mixture of azide (14.81 g, 61.1 mol) and Pd/C (0.5 g) in EtOAc/EtOH (200 mL) was stirred at 20° C. under hydrogen (60 psi) for 1 h. The mixture was filtered through celite, the cake washed with EtOAc (3×30 mL) and the solvent evaporated to give 4 (12.82 g, 97%) as a white solid, mp (EtOAc) 89-91° C.; ¹H NMR δ 4.65 (br s, 1H, OCONH), 3.52 (br s, 2H, NH₂), 2.69 (t, J=6.9 Hz, 2H, CH₂N), 1.88 (br s, 2H, CH₂N), 1.44-1.50 [m, 13H, 2×CH₂, C(CH₃)₃], 1.29-1.35 (m, 4H, 2×CH₂); ¹³C NMR δ 156.0, 78.9, 41.9, 40.4, 33.4, 29.9, 28.3 (3), 26.5, 26.4.

3-[(6-t-Butyloxycarbamoylhexyl)amino]-1,2,4-benzotriazine 1-oxide (5). A solution of amine 4 (12.8 g, 61.1 mmol) in DCM was added to a stirred solution of chloride 3 (3.70 g, 20.4 mmol) and Et₃N (5.7 mL, 40.8 mmol) in DCM (100 mL) and the solution stirred at 20° C. for 96 h. The solvent was evaporated and the residue purified by chromatography, eluting with a gradient (30-100%) of EtOAc/pet. ether, to give 1-oxide 5 (4.77 g, 65%) as a yellow powder, mp (EtOAc/pet. ether) 154-156° C.; ¹H NMR δ 8.26 (d, J=8.6 Hz, 1H, H-8), 7.70 (dd, J=8.2, 7.2 Hz, 1H, H-6), 7.59 (d, J=8.5 Hz, 1H, H-5), 7.27 (dd, J=8.0, 7.5 Hz, 1H, H-7), 5.34 (br s, 1H, NH), 4.55 (br s, 1H, OCONH), 3.51 (dd, J=6.8, 6.6 Hz, 2H, CH₂N), 3.10-3.13 (m, 2H, CH₂N), 1.64-1.72 (m, 2H, CH₂), 1.48-1.54 (m, 2H, CH₂), 1.44 [s, 9H, C(CH₃)₃], 1.38-1.43 (m, 4H, 2×CH₂); ¹³C NMR δ 158.9, 155.5, 148.9, 135.5, 130.8, 126.4, 124.8, 120.4, 79.0, 41.2, 40.3, 30.0, 29.2, 28.4 (3), 26.4, 26.3. Anal. calcd for C₁₈H₂₇N₅O₃: C, 59.8; H, 7.5; N, 19.4; found: C, 59.6; H, 7.7; N, 19.2%.

3-[(6-t-Butyloxycarbamoylhexyl)amino]-1,2,4-benzotriazine 1,4-dioxide (6). A solution of MCPBA (1.48 g, 6.02 mmol) in DCM (20 mL) was added dropwise to a stirred solution of 1-oxide 5 (1.45 g, 4.01 mmol) in DCM (100 mL) at 20° C. and the solution stirred for 4 h. The solution was partitioned between DCM (200 mL) and saturated KHCO₃ solution (200 mL). The organic fraction was dried and the solvent evaporated. The residue was purified by chromatography on neutral alumina, eluting with 50% EtOAc/DCM then a gradient (0-10%) MeOH/CHCl₃, to give (i) starting material 5 (0.73 g, 50%), and (ii) 1,4-dioxide 6 (0.55 g, 37%) as a yellow powder, mp (EtOAc/DCM) 132-134° C.; IR (KBr)) υ 3367, 3260, 1688, 1622, 1362, 1173 cm⁻¹; NMR [(CD₃)₂SO] δ 8.30 (dd, J=6.3, 6.1 Hz, 1H, OCONH), 8.19 (d, J=8.5 Hz, 1H, H-8), 8.12 (d, J=8.5 Hz, 1H, H-5), 7.91-7.95 (m, 1H, H-6), 7.53-7.57 (m, 1H, H-7), 6.76 (br s, 1H, NH), 3.32-3.39 (m, 2H, CH₂N), 2.87-2.92 (m, 2H, CH₂N), 1.56-1.61 (m, 2H, CH₂), 1.32-1.40 [m, 13H, 2×CH₂, C(CH₃)₃], 1.25-1.31 (m, 2H, CH₂); ¹³C NMR [(CD₃)₂SO] δ 155.5, 149.7, 138.1, 135.4, 129.8, 126.8, 121.1, 116.8, 77.2, 40.6, 39.8, 29.4, 28.6, 28.2 (3), 25.9, 25.8. Anal. calcd for C₁₈H₂₇N₅O₄.¼H₂O: C, 56.6; H, 7.3; N, 18.3; found: C, 56.8; H, 7.3; N, 16.8%.

N¹-(1,4-dioxido-1,2,4-benzotriazin-3-yl)-1,6-hexanediamine (7). HCl gas was bubbled through a solution of carbamate 6 (204 mg, 0.54 mmol) in MeOH (20 mL) for 2 minutes and the solution stirred at 20° C. for 16 h. The solvent was evaporated and the residue partitioned between CHCl₃ (100 mL) and saturated KHCO₃ solution (100 mL). The aqueous fraction was further extracted with CHCl₃ (3×30 mL), the combined organic extracts dried, and the solvent evaporated to give amine 7 (127 mg, 85%) as a red powder, mp 120-122° C., IR (KBR) υ 3250, 2926, 1616, 1599, 1410, 1356, 1078 cm⁻¹; ¹NMR δ 8.34 (d, J=8.5 Hz, 1H, H-8′), 8.29 (d, J=8.6 Hz, 1H, H-5′), 7.87-7.90 (m, 1H, H-6′), 7.48-7.52 (m, 1H, H-7′), 7.13 (s, 1H, NH), 3.60 (t, J=7.1 Hz, 2H, CH₂N), 2.70 (t, J=6.8 Hz, 2H, CH₂N), 1.70-1.76 (m, 2H, CH₂), 1.35-1.50 (m, 6H, 3×CH₂); ¹³C NMR [(CD₃)₂SO] δ 149.7, 138.1, 135.4, 129.8, 126.7, 121.0, 116.8, 41.5, 40.6, 33.1, 28.7, 26.1, 26.0. Anal. calcd for C₁₃H₁₉N₅O₂: C, 56.3; H, 6.9; N, 25.3; found: C, 56.3; H, 6.8; N, 22.2%. The compound was dissolved in MeOH, treated with HCl gas, and the solvent evaporated. The residue was crystallized to give the dihydrochloride of 7 as a red powder, mp (MeOH/EtOAc) 150° C. (dec.).

N¹-(1-Oxido-1,2,4-benzotriazin-3-yl)-1,6-hexanediamine (8). HCl gas was bubbled through a solution of carbamate 5 (1.0 g, 2.77 mmol) in MeOH (80 mL) for 2 minutes and the solution stirred at 20° C. for 16 h. The solvent was evaporated and the residue partitioned between CHCl₃ (100 mL) and Na₂CO₃ solution (100 mL). The aqueous fraction was further extracted with CHCl₃ (3×30 mL), the combined organic extracts dried, and the solvent evaporated to give amine 8 (0.63 g, 87%) as a red powder, mp 132-134° C., ¹H NMR δ 8.25 (dd, J=8.6, 1.0 Hz, 1H, H-8′), 7.66-7.71 (m, 1H, H-6′), 7.59 (d, J=8.4 Hz, 1H, H-5′), 7.26-7.30 (m, 1H, H-7′), 5.48 (br s, 1H, NH), 3.52 (dd, J=6.9, 6.3 Hz, 2H, H-1), 2.69 (dd, J=6.8, 6.6 Hz, 2H, H-6), 1.64-1.71 (m, 2H, H-2), 1.35-1.48 (m, 8H, H-3, H-4, H-5, NH₂); ¹³C NMR δ 159.0, 148.9, 135.5, 130.8, 126.4, 124.7, 120.4, 42.0, 41.3, 33.6, 29.3, 26.6, 26.5. Anal. calcd for C₁₃H₁₉N₅O: C, 59.7; H, 7.3; N, 26.8; found: C, 59.5; H, 7.5; N, 26.5%.

Oxidation of N¹-(1-oxido-1,2,4-benzotriazin-3-yl)-1,6-hexanediamine (8). Trifluoroacetic anhydride (11.9 mL) was added to a stirred solution of amine 8 (1.1 g, 4.2 mmol) in DCM (100 mL) and the solution stirred at 20 C for 30 min. The solution was cooled to 5° C. and 35% H₂O₂ (11.9 mL, ca 105 mmol) added dropwise and the mixture stirred vigorously for 16 h. The mixture was concentrated to 30 mL (CAUTION) and partitioned between DCM (100 mL) and sat. aqueous KHCO₃ solution (50 mL). The aqueous fraction was extracted with DCM (3×50 mL), the combined organic fraction dried and the solvent evaporated (CAUTION). The residue was purified by chromatography, eluting with a gradient (0-10%) MeOH/(40-0%) EtOAc/DCM, to give (i) 2,2,2-trifluoro-N-{6-[(1-oxido-1,2,4-benzotriazin-3-yl)amino]hexyl}acetamide (9) (0.77 g, 51%) as a yellow solid, mp (EtOAc/DCM) 188-189° C.; IR (KBr) υ 3306, 1699, 1588, 1570, 1176 cm⁻¹; ¹H NMR δ 8.27 (dd, J=8.7, 1.3 Hz, 1H, H-8′), 7.70 (ddd, J=8.5, 6.9, 1.3 Hz, 1H, H-6′), 7.59 (d, J=8.5 Hz, 1H, H-5′), 7.29 (ddd, J=8.7, 6.9, 1.3 Hz, 1H, H-7′), 6.33 (br s, 1H, NH), 5.22 (s, 1H, CONH), 3.51 (q, J==6.9 Hz, 2H, H-1), 3.38 (q, J=6.8 Hz, 2H, H-6), 1.66-1.73 (m, 2H, H-5), 1.59-1.65 (m, 2H, H-2), 1.40-1.47 (m, 4H, H-3, H-4); ¹³C NMR δ 158.7, 156.8 (q, J=37 Hz), 148.6, 135.0, 130.2, 126.0, 124.1, 119.8, 115.7 (q, J=288 Hz), 40.6, 39.2, 28.6, 28.2, 25.9, 25.8. Anal. calcd for C₁₅H₁₈F₃N₅O₂: C, 50.4; H, 5.1; N, 19.6; found: C, 50.7; H, 4.9; N, 19.6%, and: (ii) N-{6-[(1,4-dioxido-1,2,4-benzotriazin-3-yl)amino]hexyl}-2,2,2-trifluoroacetamide (10) (346 mg, 22%) as a red solid, mp (MeOH/DCM) 163-165° C.; IR (KBr) υ 3437, 3266, 1699, 1634, 1175 cm¹; ¹H NMR [(CD₃)₂SO] δ 9.42 (s, 1H, NH), 8.31 (t, J=6.0 Hz, 1H, CONH), 8.20 (d, J=8.7 Hz, 1H, H-8′), 8.12 (d, J=8.6 Hz, 1H, H-5′), 7.91-7.95 (m, 1H, H-6′), 7.53-7.57 (m, 1H, H-7′), 3.40 (q, J=6.7 Hz, 2H, H-1), 3.18 (q, J=6.6 Hz, 2H, H-6), 1.58-1.64 (m, 2H, H-2), 1.46-1.53 (m, 2H, H-5), 1.28-1.38 (m, 4H, H-3, H-4) ¹³C NMR [(CD₃)₂SO] δ 156.0 (q, J=36 Hz), 149.7, 138.1, 135.4, 129.8, 126.7, 121.1, 116.8, 115.9 (q, J=288 Hz), 40.5, 39.0, 28.5, 28.1, 25.8, 25.7. Anal. calcd for C₁₅H₁₈F₃N₅O₃: C, 48.3; H, 4.9; N, 18.8; found: C, 48.5; H, 4.7; N, 18.0%.

Oxidation of 2,2,2-trifluoro-N-{6-[(1-oxido-1,2,4-benzotriazin-3-yl)amino]hexyl}acetamide (9). Trifluoroacetic anhydride (4.0 mL, 28.6 mmol) was added dropwise to a stirred suspension of 35% H₂O₂ (2.2 mL, ca. 23 mmol) in DCM (20 mL) at 5° C. and the mixture was stirred for 15 min. The mixture was dried and added to a stirred solution of 1-oxide 9 (409 mg, 1.14 mmol) in DCM (50 mL) and the solution stirred at 20° C. for 48 h. The solution was partitioned between sat. aqueous KHCO₃ (50 mL) and CHCl₃ (50 mL). The aqueous fraction was extracted with CHCl₃ (3×40 mL), the combined organic fraction dried, and the solvent evaporated (CAUTION). The residue was purified by chromatography, eluting with a gradient (0-10%) MeOH/(40-0%) EtOAc/DCM, to give (i) starting material 9 (250 mg, 61%); and (ii) 1,4-dioxide 10 (124 mg, 29%), spectroscopically identical to a sample obtained above.

N¹-(1,4-Dioxido-1,2,4-benzotriazin-3-yl)-1,6-hexanediamine (7). 1 M NaOH solution (2.8 mL, 2.8 mmol) was added to a stirred solution of trifluoroacetamide 10 (209 mg, 0.56 mmol) in MeOH (20 mL) and the mixture stirred at 20° C. for 16 h. The solvent was evaporated and the residue partitioned between sat. aqueous KHCO₃ (70 mL) and CHCl₃ (70 mL). The aqueous fraction was extracted with CHCl₃ (3×30 mL), the combined organic fraction dried, and the solvent evaporated to give amine 7 (129 mg, 83%), spectroscopically identical with the sample obtained above.

### Example B

N¹-(9-Acridinyl)-N⁶-(1,4-dioxido-1,2,4-benzotriazin-3-yl)-1,6-hexanediamine (11). A solution of amine 7 (64 mg, 0.23 mmol) and 9-methoxyacridine (53 mg, 0.25 mmol) in MeOH (10 mL) was stirred at reflux temperature for 10 h. The solvent was evaporated and the residue purified by chromatography on neutral alumina, eluting with a gradient (0-5%) of MeOH/CHCl₃, to give compound 11 (63 mg, 60%) as a red solid, IR (KBr) υ 3293, 1593, 1414, 1362 cm⁻¹; ¹H NMR δ 8.30 (d, J=8.5 Hz, 1H, H-8″), 8.29 (d, J=8.5 Hz, 1H, H-5″), 8.11 (d, J=8.6 Hz, 2H, H-1′, H-8′), 8.04 (d, J=8.6 Hz, 2H, H-4′, H-5′), 7.84 (ddd, J=8.5, 7.2, 1.2 Hz, 1H, H-6″), 7.59-7.64 (m, 2H, H-3′, H-6′), 7.57 (ddd, J=8.5, 7.2, 1.2 Hz, 1H, H-7″), 7.31-7.35 (m, 2H, H-2′, H-7′), 7.15 (br s, 1H, NH), 3.84 (dd, J=7.2, 7.1 Hz 2H, CH₂N), 3.57 (dt, J=6.7, 6.5 Hz, 2H, CH₂N), 1.78-1.85 (m, 2H, CH₂), 1.67-1.74 (m, 2H, CH₂), 1.43-1.53 (m, 4H, 2×CH₂), NH not observed, ¹³C NMR δ 151.9 (2), 149.8, 148.0, 138.1, 135.8, 130.3, 130.2 (2), 128.1 (2), 127.1, 123.0 (2), 122.9 (2), 121.6, 117.2, 116.1 (2), 50.4, 41.2, 31.4, 29.2, 26.4, 26.3; MS (FAB⁺) m/z 455 (MH⁺, 20%), 439 (10); HRMS (FAB⁺) calcd for C₂₆H₂₇N₆O₂ (MH+) m/z 455.2196, found 455.2182. The compound was dissolved in MeOH and treated with HCl gas and the solvent evaporated. The residue was crystallized from MeOH/EtOAc to give the hydrochloride of 11, mp (MeOH/EtOAc) 118-119° C. Anal. calcd for C₂₆H₂₆N₆O₂.2HCl.½H₂O: C, 58.2; H, 5.5; N, 15.7; found: C, 57.8; H, 5.5; N, 15.3%.

### Example C

N-{6-[(1,4-Dioxido-1,2,4-benzotriazin-3-yl)amino]hexyl}-4-acridinecarboxamide (12). A solution of the amine 7 (447 mg, 1.6 mmol) in THF (20 mL) and DMF (10 mL) was added dropwise to a stirred solution of acridine-4-carboxylic acid imidazolide (440 mg, 1.61 mmol) in THF (20 mL) at 5° C. and the solution Bred at 20° C. for 16 h. The solvent was evaporated and the residue purified by chromatography, eluting with a gradient (0-5%) of MeOH/DCM, to give compound 12 (708 mg, 91%) as a red solid, mp (EtOAc) 196-198° C.; ¹H NMR δ 11.88 (s, 1H, NH, 8.99 (dd, J=7.1, 1.5 Hz, 1H, H-3), 8.90 (s, 1H, H-9), 8.30 (d, J=8.4 Hz, 1H, H-8′), 8.28 (d, J=8.7 Hz, 1H, H-1′), 8.17 (d, J=9.1 Hz, 1H, H-5), 8.14 (dd, J=8.4, 1.5 Hz, 1H, H-1), 8.04 (d, J=8.1 Hz, 1H, H-8), 7.84-7.91 (m, 2H, H-6, H-6′), 7.66 (dd, J=8.3, 7.2 Hz, 1H, H-2), 7.59 (ddd, J=7.9, 7.0, 0.9 Hz, 1H, H-7), 7.59 (ddd, J=8.4, 7.2, 1.2 Hz, 1H, H-7′), 7.11 (br dd, J=5.7, 5.5 Hz, 1H, CONH), 3.71 (dd, J=6.9, 5.6 Hz, 2H, CH₂N), 3.63 (dd, J=6.9, 6.7 Hz, 2H, CH₂N), 1.83-1.89 (m, 2H, CH₂), 1.74-1.81 (m, 2H, CH₂), 1.55-1.68 (m, 4H, 2×CH₂); ¹³C NMR δ 164.6, 149.7, 147.0, 145.5, 145.0, 138.7, 138.1, 135.4, 134.5, 132.8, 132.0, 129.8, 128.5, 128.3, 126.8, 126.5, 126.4, 125.6, 125.3, 121.1, 116.8, 40.6, 39.0, 29.0, 29.6, 26.5, 26.0. Anal. calcd for C₂₇H₂₆N₆O₀.H₂O: C, 64.8; H, 5.6; N, 16.8; found: C, 65.0; H, 5.5; N, 17.1%.

### Example D

N-{6-[(1,4-Dioxido-1,2,4-benzotriazin-3-yl)amino]hexyl}-4-quinolinecarboxamide (13). A solution of 4-quinolinecarboxylic acid (308 mg, 1.78 mmol) and CDI (346 mg, 2.13 mmol) in DMF (20 mL) were stirred at 50° C. for 1 h. The solvent was evaporated and the residue recrystallised from DCM/pet ether to give 4-(1H-imidazol-1-ylcarbonyl)quinoline which was used directly without characterisation. A solution of the amine 7 (494 mg, 1.78 mmol) in DMF (10 mL) was added dropwise to a stirred solution of imidazolide in THF (20 mL) at 5° C. and the solution was stirred at 20° C. for 16 h. The solvent was evaporated and the residue purified by chromatography, eluting with a gradient (0-5%) of MeOH/DCM, to give compound 13 (619 mg, 80%) as a red powder, mp (MeOH/DCM) 196-198° C.; ¹H NMR [(CD₃)₂SO] δ 8.96 (d, J=4.3 Hz, 1H, H-2), 8.75 (t, J=5.5 Hz, 1H, CONH), 8.32 (t, J=6.1 Hz, 1H, NH), 8.20 (d, J=8.6 Hz, 1H, H-8″), 8.12 (d, J=8.6 Hz, 1H, H-5″), 8.11 (d, J=8.7 Hz, 1H, H-5), 8.06 (d, J=8.4 Hz, 1H, H-8), 7.92 (ddd, J=8.4, 7.1, 1.3 Hz, 1H, H-6″), 7.80 (ddd, J=8.4, 7.1, 1.0 Hz, 1H, H-7), 7.66 (ddd, J=8.5, 7.0, 1.0 Hz, 1H, H-6), 7.55 (ddd, J=8.5, 7.1, 1.3 Hz, 1H, H-7″), 7.52 (d, J=4.4 Hz, 1H, H-3), 3.39-3.43 (m, 2H, H-1′), 3.3-3.35 (m, 2H, H-6′), 1.65-1.70 (m, 2H, H-2′), 1.56-1.73 (m, 2H, H-5′), 1.38-1.45 (m, 4H, H-3′, H-4′); ¹³C NMR [(CD₃)₂SO] δ 166.4, 150.2, 149.7, 147.8, 142.4, 138.1, 135.4, 129.8, 129.6, 129.2, 127.1, 126.7, 125.3, 124.1, 121.0, 118.8, 116.7, 40.6, 38.9, 28.8, 28.6, 26.1, 25.9. Anal. calcd for C₂₃H₂₄N₆O₃: C, 63.9; H, 5.6; N, 19.4; found: C, 63.9; H, 5.4; N, 19.5%.

### Example E

### N-{3-[(1,4-Dioxido-1,2,4-benzotriazin-3-yl)amino]propyl}-4-acridinecarboxamide (17)

tert-Butyl 3-[(1-oxido-1,2,4-benzotriazin-3-yl)amino]propylcarbamate (14). A solution of chloride 3 (4.0 g, 22.0 mmol), tert-butyl 3-aminopropylcarbamate (5.76 g, 33.0 mmol) and Et₃N (4.6 mL, 33.0 mmol) in DCM (150 mL) was stirred at 20° C. for 5 d. The solvent was evaporated, and the residue purified by chromatography, eluting with 20% EtOAc/DCM, to give 1-oxide 14 (5.21 g, 74%) as a yellow powder, mp (EtOAc/DCM) 145-147° C.; ¹H NMR [(CD₃)₂SO] δ 8.13 (dd, J=8.6, 1.1 Hz, 1H, H-8′), 7.84 (s, 1H, NH), 7.78 (ddd, J=8.4, 7.1, 1.1 Hz, 1H, H-6′), 7.56 (d, J=8.4 Hz, 1H, H-5′), 7.32 (ddd, J=8.6, 7.1, 1.1 Hz, 1H, H-7′), 6.83 (t, J=5.3 Hz, 1H, NHCO₂), 3.32-3.36 (m, 2H, H-1), 2.99-3.04 (m, 2H, H-3), 1.66-1.73 (m, 2H, H-2), 1.37 [s, 9H, C(CH₃)₃]; ¹³C NMR [(CD₃)₂SO] δ 158.9, 155.6, 148.2, 135.7, 130.0, 125.9, 124.4, 119.9, 77.4, 38.2, 37.5, 28.9, 28.2 (3). Anal. calcd for C₁₅H₂₁N₅O₃: C, 56.4; H, 6.6; N, 21.9; found: C, 56.4; H, 6.6; N, 22.1%.

tert-Butyl 3-[(1,4-dioxido-1,2,4-benzotriazin-3-yl)amino]propylcarbamate (15). A solution of MCPBA (6.74 g, 27.3 mmol) in DCM (80 mL) was added dropwise to a stirred solution of 1-oxide 14 (5.82 g, 18.2 mmol) in DCM (300 mL) and NaHCO₃ (3.1 g, 36.5 mmol). The mixture was stirred at 20° C. for 1 h, partitioned between DCM (400 mL) and sat. aqueous KHCO₃ solution (100 mL). The organic fraction was dried and the solvent evaporated. The residue was purified by chromatography, eluting with a gradient (0-10%) of MeOH/40% EtOAc/DCM, to give (i) starting material 14 (2.63 g, 45%) and (ii) 1,4-dioxide 15 (1.47 g, 24%) as a red solid, mp (EtOAc/MeOH) 134-136° C.; ¹H NMR [(CD₃)₂SO] δ 8.30 (t, J=6.2 Hz, 1H, NH), 8.20 (d, J=8.5 Hz, 1H, H-8′), 8.13 (d, J=8.5 Hz, 1H, H-5′), 7.93 (ddd, J=8.5, 7.1, 1.3 Hz, 1H, H-6′), 7.57 (ddd, J=8.5, 7.1, 1.3 Hz, 1H, H-7′), 6.86 (t, J=5.6 Hz, 1H, NHCO₂), 3.38-3.42 (m, 2H, H-1), 2.98-3.02 (m, 2H, H-3), 1.68-1.74 (m, 2H, H-2), 1.37 [s, 9H, C(CH₃)₃]; ¹³C NMR [(CD₃)₂SO] δ 155.6, 149.7, 138.1, 135.4, 129.8, 126.8, 121.0, 116.8, 77.4, 38.2, 37.1, 28.9, 28.1 (3). Anal. calcd for C₁₅H₂₁N₅O₄.¼EtOAc: C, 53.8; H, 6.5; N, 19.6; found: C, 53.5; H, 6.5; N, 19.5%.

N¹-(1,4-Dioxido-1,2,4-benzotriazin-3-yl)-1,3-propanediamine (16). HCl saturated MeOH (20 mL) was added to a solution of carbamate 15 (1.47 mg, 4.38 mmol) in MeOH (30 mL) and the solution stirred at 20° C. for 16 h. The solution was evaporated and the residue dissolved in water (20 mL) the solution neutralized with KHCO₃ and extracted with CHCl₃ (5×50 mL). The combined organic fraction was dried and the solvent evaporated to give compound 16 (0.82 g, 80%) as a red solid, mp (MeOH) 121-123° C.; ¹H NMR [(CD₃)₂SO] δ 8.24 (d, J=8.4 Hz, 1H, H-8′), 8.13 (d, J=8.6 Hz, 1H, H-5′), 7.99 (ddd, J=8.6, 7.1, 1.0 Hz, 1H, H-6′), 7.61 (ddd, J=8.4, 7.1, 1.0 Hz, 1H, H-7′), 4.01 (br s, 3H, NH, NH₂), 3.48 (t, J=6.7 Hz, 2H, H-1), 2.66 (t, J=7.0 Hz, 2H, H-3), 1.73-1.77 (m, 2H, H-2); MS (FAB⁺) m/z 236 (MH⁺, 6%), 220 (10), 204 (5); HRMS calcd for C₁₀H₁₄N₅O₂ (MH⁺) m/z 236.1148, found 236.1139.

N-{3-[(1,4-Dioxido-1,2,4-benzotriazin-3-yl)amino]propyl}-4-acridinecarboxamide (17). A solution of amine 16 (128 mg, 0.54 mmol) in DCM (5 mL) was added dropwise to a stirred solution of 4-(1H-imidazol-1-ylcarbonyl)acridine (156 mg, 0.57 mmol) in DCM (10 mL) at 5° C. and the solution was stirred at 20° C. for 6 d. The solvent was evaporated and the residue purified by chromatography, eluting with a gradient (0-5%) of MeOH/DCM, to give compound 17 (102 mg, 80%) as a red gum, ¹H NMR [(CD₃)₂SO] δ 11.39 (t, J=5.5 Hz, 1H, CONH), 9.31 (s, 1H, H-9), 8.71 (dd, J=7.1, 1.4 Hz, 1H, H-3), 8.48 (br s, 1H, NH), 8.38 (dd, J=8.4, 1.4 Hz, 1H, H-1), 833 (d, J=9.2 Hz, 1H, H-5), 8.22 (d, J=8.5 Hz, 1H, H-8), 8.13 (d, J=8.4 Hz, 1H, H-8′), 8.06 (d, J=8.7 Hz, 1H, H-5′), 7.87-7.95 (m, 2H, H-6, H-6′), 7.76 (dd, J=8.4, 7.1 Hz, 1H, H-2), 7.68 (dd, J=8.5, 7.2 Hz, 1H, H-7), 7.54 (ddd, J=8.5, 7.1, 1.3 Hz, 1H, H-7′), 3.64-3.70 (m, 4H, 2 CH₂N), 2.05-2.10 (m, 2H, CH₂); ¹³C NMR (CD₃OD) δ 168.9, 152.2, 151.5 (2), 141.0, 140.5, 140.1 (2), 138.8, 137.9, 135.7, 133.7, 131.5, 130.1, 128.9, 128.5, 128.1, 127.0, 123.0, 121.9, 121.6, 40.6, 38.1, 29.6. An analytical sample was recrystallized as the dihydrochloride salt, mp (MeOH/EtOAc) 192° C. Anal. calcd for C₂₄H₂₀N₆O₃.2HCl.½H₂O: C, 55.2; H, 4.4; N, 16.1; found: C, 55.3; H, 4.5; N, 16.1%.

### Example F

### N-(2-{2-[(1,4-Dioxido-1,2,4-benzotriazin-3-yl)amino]ethoxy}ethyl)-4-acridinecarboxamide (23)

3-{[2-(2-Hydroxyethoxy)ethyl]amino}-1,2,4-benzotriazine 1-oxide (18). A solution of chloride 3 (3.0 g, 16.52 mmol) in DCM (50 mL) was added to a stirred solution of 2-(aminoethoxy)ethanol (2.49 mL, 24.8 mmol) and Et₃N (3.45 mL, 24.8 mmol) in DCM (80 mL) and the solution stirred at 20° C. for 16 h. The solvent was evaporated and the residue purified by chromatography, eluting, with 40% EtOAc/DCM, to give 1-oxide 18 (2.62 g, 63%) as a yellow powder, mp (DCM/EtOAc) 131-131.5° C.; ¹H NMR δ 8.25 (dd, J=8.7, 1.2 Hz, 1H, H-8), 7.68 (ddd, J=8.4, 7.2, 1.5 Hz, 1H, H-6), 7.57 (d, J=8.4 Hz, 1H, H-5), 7.28 (ddd, J=8.7, 7.2, 1.3 Hz, 1H, H-7), 6.02 (br s, 1H, NH), 3.74-3.80 (m, 6H, 3×CH₂O), 3.64-3.67 (m, 2H, CH₂N), 2.71 (t, J=5.9 Hz, 1H, OH); ¹³C NMR δ 158.9, 149.7, 135.5, 130.9, 126.4, 124.9, 120.4, 72.4, 69.5, 61.7, 41.9. Anal. calcd for C₁₁H₁₄N₄O₃: C, 52.8; H, 5.6; N, 22.4; found: C, 52.9; H, 5.7; N, 22.6%.

3-{[2-(2-Azidoethoxy)ethyl]amino}-1,2,4-benzotriazine 1-oxide (19). Methanesulfonyl chloride (0.82 mL, 10.6 mmol) was added dropwise to a stirred solution of alcohol 18 (2.41 g, 9.63 mmol) and Et₃N (1.74 mL, 12.5 mmol) in DCM (100 mL) at 5° C. and the solution stirred at 20° C. for 1 h. The solution was diluted with DCM (100 mL) and washed with water (3×50 mL), brine (50 mL), dried and the solvent evaporated. The residue was dissolved in DMF (50 mL) and NaN₃ (0.69 g, 10.6 mmol) added. The mixture was heated at 100° C. for 2 h. cooled to 30° C. and the solvent evaporated. The residue was partitioned between EtOAc (100 mL) and water (100 mL). The organic fraction was washed with brine (50 mL), dried, and the solvent evaporated. The residue was purified by chromatography, eluting with 50% EtOAc/pet. ether, to give azide 19 (2.35 g, 89%) as yellow crystals, mp (EtOAc/pet. ether) 102-104° C.; ¹H NMR δ 8.27 (dd, J=8.7, 1.4 Hz, 1H, H-8), 7.70 (ddd, J=8.6, 7.1, 1.5 Hz, 1H, H-6), 7.59 (d, J=8.6 Hz, 1H, H-5), 7.29 (ddd, J=8.6, 7.1, 1.4 Hz, 1H, H-7), 5.70 (br s, 1H, NH), 3.71-3.78 (m, 4H, 2×CH₂O), 3.69 (dd, J=5.3, 4.8 Hz, 2H, CH₂N₃), 3.41 (dd, J=5.1, 4.9 Hz, 2H, CH₂N); ¹³C NMR δ 158.9, 148.7, 135.5, 131.1, 126.5, 125.0, 120.4, 70.0, 69.6, 50.7, 41.1. Anal. calcd for C₁₁H₁₃N₇O₂; C, 48.0; H, 4.8; N, 35.6; found: C, 48.3; H, 4.6; N, 35.7%.

3-{[2-(2-tert-Butyloxycarbamoylethoxy)ethyl]amino}-1,2,4-benzotriazine 1-oxide (20). Propane-1,3-dithiol (5.7 mL, 57.0 mmol) was added dropwise to a stirred solution of azide 19 (1.57 g, 5.70 mmol) and Et₃N (7.95 mL, 57 mmol) in MeOH (100 mL) under N₂ and the solution heated at reflux temperature for 8 h. The solution was cooled to 30° C. and partitioned between 1 M HCl (100 mL) and Et₂O (100 mL). The aqueous fraction was adjusted to pH 12 with 7 M NaOH solution and extracted with DCM (3×50 mL). The organic fraction was dried and the solvent evaporated. The residue was dissolved in THF (100 mL) and a solution of di-tert-butyldicarbonate (1.87 g, 8.55 mmol) in THF (50 ml) added dropwise. The solution was stirred at 20° C. for 16 h, the solvent evaporated and the residue purified by chromatography, eluting with 40% EtOAc/pet. ether, to give carbamate 20 (1.85 g, 93%) as a yellow solid, mp (EtOAc/pet. ether) 134-137° C.; ¹H NMR δ 8.26 (dd, J=8.4, 0.9 Hz, 1H, H-8), 7.71 (ddd, J=8.3, 7.1, 1.4 Hz, 1H, H-6), 7.59 (d, J=8.3 Hz, 1H, H-5), 7.29 (ddd, J=8.4, 7.1, 1.3 Hz, 1H, H-7), 5.74 (br s, 1H, NH), 4.93 (br s, 1H, NH), 3.67-3.73 (m, 4H, 2×CH₂O), 3.56 (t, J=5.2 Hz, 2H, CH₂N), 3.29-3.36 (m, 2H, CH₂N), 1.45 [s, 9H, C(CH₃)₃]; ¹³C NMR δ 159.9, 155.9, 148.7, 135.5, 131.0, 126.5, 125.0, 120.4, 79.4, 70.2, 69.2, 41.1, 40.4, 28.4 (3). Anal. calcd for C₁₆H₂₃N₅O₄: C, 55.0; H, 6.6; N, 20.1; found: C, 55.3; H, 6.8; N, 20.1%.

3-{[2-(2-tert-Butyloxycarbamoylethoxy)ethyl]amino}-1,2,4-benzotriazine 1,4-dioxide (21). A solution of MCPBA (1.57 g, 6.35 mmol) in DCM (50 mL) was added dropwise to a stirred solution of carbamate 20 (1.85 g, 5.29 mmol) in DCM (100 mL) and NaHCO₃ (0.89 g, 10.6 mmol) and the mixture was stirred at 20° C. for 6 h. The suspension was filtered through celite, the solvent evaporated and the residue purified by chromatography, eluting with a gradient of (0-5%) MeOH/(40-0%) EtOAc/DCM, to give (i) starting material 20 (926 mg, 50%), spectroscopically identical with an authentic sample, and (ii) 1,4-dioxide 21 (702 mg, 40%) as a red solid, mp (EtOAc) 139-140° C.; ¹H NMR δ 8.33 (d, J=8.7 Hz, 1H, H-8), 8.30 (d, J=8.7 Hz, 1H, H-5), 7.88 (ddd, J=8.7, 7.2, 1.2 Hz, 1H, H-6), 7.43-7.50 (m, 2H, H-7, NH), 5.06 (br s, 1H, NH), 3.78-3.83 (m, 2H, CH₂O), 3.69 (dd, J=5.1, 5.0 Hz, 2H, CH₂O), 3.56 (dd, J=5.1, 5.0 Hz, 2H, CH₂N), 3.29-3.36 (m, 2H, CH₂N), 1.43 [s, 9H, C(CH₃)₃]; ¹³C NMR δ 156.0, 149.8, 138.5, 135.9, 130.6, 129.5, 121.6, 117.4, 79.4, 70.3, 68.9, 41.3, 40.3, 28.3 (3); MS (FAB⁺) m/z 366 (MH⁺, 40%), 350 (5) 310 (20); HRMS (FAB⁺) calcd for C₁₆H₂₄N₅O₅ (MH⁺) m/z 366.1777, found 366.1767. Anal. calcd for C₁₆H₂₃N₅O₅.½H₂O: C, 51.3; H, 6.5; N, 18.7; found: C, 51.3; H, 6.2; N, 16.9%.

3-{[2-(2-Aminoethoxy)ethyl]amino}-1,2,4-benzotriazine 1,4-dioxide (22). Trifluoroacetic acid (1.66 mL, 34.6 mmol) was added dropwise to a stirred solution of 1,4-dioxide 21 (632 mg, 1.73 mmol) in DCM (50 mL) and the solution stirred at 20° C. for 16 h. The solvent was evaporated and the residue partitioned between sat. aqueous KHCO₃ solution (100 mL) and CHCl₃ (100 mL). The aqueous phase was extracted with CHCl₃ (8×50 mL), the combined organic fractions dried, and the solvent evaporated. The residue was crystallized from CHCl₃ to give the amine 22 (406 mg, 91%) as a red solid, mp (CHCl₃) 124° C. (dec.); ¹H NMR δ 8.26 (d, J=8.9 Hz, 1H, H-8), 8.23 (d, J=8.9 Hz, 1H, H-5), 7.79 (dd, J=8.8, 7.8 Hz, 1H, H-6), 7.45 (dd, J=8.9, 7.7 Hz, 1H, H-7), 3.75 (dd, J=5.0, 4.8 Hz, 2H, CH₂O), 3.66 (dd, J=5.0, 4.9 Hz, 2H, CH₂O), 3.47 (dd, J=5.1, 5.0 Hz, 2H, CH₂N), 2.82 (dd, J=5.1, 5.0 Hz, 2H, CH₂N), NH and NH₂ not observed; ¹³C NMR δ 149.8, 138.3, 135.8, 130.5, 127.2, 121.6, 117.4, 73.0, 68.9, 41.7, 41.3; MS (FAB⁺) m/z 266 (MH⁺, 20%), 250 (5); HRMS (FAB⁺) calcd for C₁₁H₁₆N₅O₃ (MH⁺) m/z 266.1253, found 266.1230. Anal. calcd for C₁₁H₁₅N₅O₃.¼H₂O: C, 49.0; H, 5.8; N, 26.0; found: C, 49.0; H, 5.7; N, 24.7%.

N-(2-{2-[(1,4-Dioxido-1,2,4-benzotriazin-3-yl)amino]ethoxy}ethyl)-4-acridinecarboxamide (23). A solution of the amine 22 (54 mg, 0.20 mmol) in THF (2 mL) was added dropwise to a stirred solution of 4-(1H-imidazol-1-ylcarbonyl)acridine (58 mg, 0.21 mmol) in THF (5 mL) at 5° C. and the solution stirred at 20° C. for 16 h. The solvent was evaporated and the residue purified by chromatography, eluting with a gradient (0-5%) of MeOH/DCM, to give compound 23 (93 mg, 97%) as a red solid, mp (EtOAc) 98-100° C.; ¹H NMR δ 12.14 (s, 1H, CONH), 8.96 (dd, J=7.1, 1.5 Hz, 1H, H-3′), 8.82 (s, 1H, H-9), 8.25 (d, J=8.4 Hz, 1H, H-8′), 8.16 (d, J=8.4 Hz, 1H, H-5′), 8.11-8.13 (m, 2H, H-1, H-5), 7.94 (d, J=8.2 Hz, 1H, H-8), 7.76-7.84 (m, 2H, H-6, H-6′), 7.66 (dd, J=8.4, 7.1 Hz, 1H, H-2), 7.44-7.52 (m, 2H, H-7, H-7′), 7.36 (br s, 1H, NH), 3.85-3.95 (m, 8H, 2×CH₂O, 2×CH₂N); ¹³C NMR δ 166.1, 149.8, 147.2, 146.3, 138.1, 137.6, 135.5, 135.3, 132.4, 131.3, 130.4, 128.8, 128.3, 128.0, 127.1, 126.8, 126.2, 125.8, 125.4, 121.5, 117.3, 70.2, 68.9, 41.1, 39.5; MS (FAB⁺) m/z 471 (MH⁺, 5%), 455 (4); HRMS (FAB⁺) calcd for C₂₅H₂₃N₆O₄ (MH⁺) m/z 471.1781, found 471.1790. Anal. calcd for C₂₅H₂₂N₆O₄.½H₂O: C, 62.6; H, 4.8; N, 17.5; found: C, 63.0; H, 4.7; N, 17.5%.

### Example G

N-(2-{2-[(1,4-Dioxido-1,2,4-benzotriazin-3-yl)amino]ethoxy}ethyl)-8-quinolinecarboxamide (24). A solution of 8-quinolinecarboxylic acid (308 mg, 1.78 mmol) and CDI (346 mg, 2.13 mmol) in DMF (20 mL) were stirred at 50° C. for 1 h. The solvent was evaporated and the residue recrystallised from DCM/pet. ether to give 4-(1H-imidazol-1-ylcarbonyl)quinoline (50 mg, 0.21 mmol) which was used directly without characterisation. A solution of the amine 22 (57 mg, 0.21 mmol) in DCM (10 mL) was added dropwise to a stirred solution of imidazolide (50 mg, 0.21 mmol) in DCM (5 mL) at 5° C. and the solution was stirred at 20° C. for 16 h. The solvent was evaporated and the residue purified by chromatography, eluting with a gradient (0-5%) of MeOH/DCM, to give compound 24 (74 mg, 84%) as a red powder, mp (MeOH/DCM) 168-170° C.; ¹H NMR δ 11.51 (br s, 1H, NH), 9.01 (dd, J=4.2, 1.9 Hz, 1H, H-2), 8.85 (dd, J=7.3, 1.6 Hz, 1H, H-4), 8.30 (d, J=8.3 Hz, 1H, H-8″), 8.23-8.26 (m, 2H, H-7, H-5″), 7.93 (dd, J=8.1, 1.5 Hz, 1H, H-5), 7.86 (ddd, J=8.4, 7.0, 1.8 Hz, 1H, H-6″), 7.67 (dd, J=7.9, 7.5 Hz, 1H, H-6), 7.46-7.51 (m, 2H, H-3, H-7″), 7.46 (br s, 1H, NH, 3.78-3.85 (m, 8H, 2×CH₂O, 2×CH₂N); ¹³C NMR δ 166.0, 149.8, 149.6, 145.6, 138.3, 137.6, 135.7, 133.8, 131.9, 130.5, 128.7, 128.4, 127.2, 126.4, 121.6, 120.9, 117.4, 70.3, 68.9, 41.4, 39.6; MS (FAB⁺) m/z 421 (MH⁺, 8%), 405 (5), 389 (1); HRMS (FAB⁺) calcd for C₂₁H₂₁N₆O₄ (MH⁺) m/z 421.1624, found 421.1615. Anal. calcd for C₂₁H₂₀N₆O₄.½MeOH: C, 59.2; H, 5.1; N, 19.3; found: C, 59.2; H, 4.8; N, 19.2%.

### Example H

N-(2-{2-[(1,4-Dioxido-1,2,4-benzotriazin-3-yl)amino]ethoxy}ethyl)-2-phenyl-1H-benzimidazole-4-carboxamide (25). A solution of 2-phenyl-1H-benzimidazole-4-carboxylic acid (396 mg, 1.67 mmol) and CDI (270 mg, 1.67 mmol) in DMF (10 mL) was stirred at 50° C. for 1 h. The solvent was evaporated and the residue recrystallised from DCM/pet. ether to give 4-(1H-imidazol-1-ylcarbonyl)-2-phenyl-1H-benzimidazole (309 mg, 0.21 mmol) which was used directly without characterisation. A solution of the amine 22 (56 mg, 0.21 mmol) in DCM (5 mL) was added dropwise to a stirred solution of imidazolide (61 mg, 0.21 mmol) in DCM (5 mL) at 5° C. and the solution was stirred at 20° C. for 16 h. The solvent was evaporated and the residue purified by chromatography, eluting with a gradient (0-5%) of MeOH/DCM, to give compound 25 (89 mg, 86%) as a red powder, mp (DCM) 203-207° C.; ¹H NMR [(CD₃)₂SO] δ 13.30 (br s, 1H, NH), 10.24 (s, 1H, NH), 8.18-8.24 (m, 3H, H-2′, H-6′, NH), 8.13 (d, J=8.7 Hz, 1H, H-8″), 8.04 (d, J=8.5 Hz, 1H, H-D″), 7.90-7.94 (m, 1H, H-6″), 7.87 (d, J=7.9 Hz, 1H, H-5), 7.72 (d, J=7.9 Hz, 1H, H-7), 7.52-7.58 (m, 3H, H-3′, H-5′, H-7″), 7.46-7.48 (m, 1H, H-4′), 7.34 (t, J=7.9 Hz, 1H, H-6), 3.78-3.82 (m, 2H, CH₂O), 3.74-3.77 (m, 2H, CH₂O), 3.63-3.78 (m, 4H, 2×CH₂N); ¹³C NMR [(CD₃)₂SO] δ 164.5, 151.7, 149.7, 141.0, 138.0, 135.4, 135.1, 130.4, 129.9, 128.9 (2), 128.8, 126.9, 126.6 (2), 122.5, 122.3, 122.0, 121.0, 116.7, 114.8, 69.1, 68.2, 40.3, 38.8; MS (FAB⁺) m/z 486 (MH⁺, 4%), 470 (2); HRMS (FAB⁺) calcd for C₂₅H₂₄N₇O₄ (MH⁺) m/z 486.1890, found 486.1903. Anal. calcd for C₂₅H₂₃N₇O₄: C, 61.8; H, 4.8; N, 20.2; found: C, 61.6; H, 4.7; N, 20.0%.

### Example I

N-(2-{2-[(1,4-Dioxido-1,2,4-benzotriazin-3-yl)amino]ethoxy}ethyl)-2-(4-pyridinyl)-8-quinolinecarboxamide (26). A solution of 2-(4-pyridinyl)-8-quinolinecarboxylic acid (268 mg, 1.07 mmol) and CDI (173 mg, 1.07 mmol) in DMF (10 mL) were stirred at 50° C. for 1 h. The solvent was evaporated and the residue recrystallized from DCM/pet. ether to give S-(1H-imidazol-1-ylcarbonyl)-2-(4-pyridinyl)quinoline (238 mg, 0.86 mmol) which was used directly without characterization. A solution of the amine 22 (39 mg, 0.15 mmol) in DCM (5 mL) was added dropwise to a stirred solution of imidazolide (41 mg, 0.15 mmol) in DCM (5 mL) at 5° C. and the solution was stirred at 20° C. for 16 h. The solvent was evaporated and the residue purified by chromatography, eluting with a gradient (0-5%) of MeOH/DCM, to give compound 26 (51 mg, 70%) as a red powder, mp (DCM) 128-130° C.; ¹H NM [(CD₃)₂SO] δ 12.01 (br s, 1H, NH, 10.83 (t, J=5.3 Hz, 1H, NH), 8.70 (dd, J=4.5, 1.5 Hz, 2H, H-3′, H-5′), 8.63 (d, J=8.6 Hz, 1H, H-7), 8.58 (dd, J=7.3, 1.5 Hz, 1H, H-4), 8.23 (d, J=8.7 Hz, 1H, H-5), 8.19 (dd, J=8.7, 1.5 Hz, H, H-8′), 8.09 (dd, J=4.5, 1.5 Hz, 2H, H-2′, H-6′), 7.95 (d, J=8.5 Hz, 1H, H-5″), 7.82-7.90(m, 2H, H-3, H-6′), 7.76(t, J=8.7 Hz, 1H, H-6), 7.47 (ddd, J=8.7, 7.0, 1.6 Hz, 1H, H-7″), 3.70-3.78 (m, 6H, 2×CH₂O, CH₂N), 3.49-3.54 (m, 2H, CH₂N); MS (FAB⁺) m/z 498 (MH⁺, 10%), 482 (5); HRMS (FAB⁺) calcd for C₂₆H₂₄N⁷O₄ (MH⁺) m/z 498.1890, found 498.1898. Anal. calcd for C₂₆H₂₃N₇O₄.H₂O: C, 60.7; H, 4.9; N, 22.0; found: C, 60.6; H, 4.9; N, 19.0%.

### Example J

### N-[3-({3-[(1,4-Dioxido-1,2,4-benzotriazin-3-yl)amino]propyl}amino)propyl]-4-acridinecarboxamide (30)

tert-Butyl 3-[(1-oxido-1,2,4-benzotriazin-3-yl)amino]propyl{3-[(trifluoroacetyl)amino]propyl}carbamate (27). A solution of chloride 3 (1.34 g, 7.41 mmol) in DCM (50 mL) was added dropwise to a stirred solution of tert-butyl bis(3-aminopropyl)carbamate (2.57 g, 11.1 mmol) and Et₃N (1.55 mL, 11.1 mol) in DCM (200 mL) at 20° C. The solution was stirred at 20° C. for 3 d. The solvent was evaporated and the residue purified by chromatography, eluting with 50% EtOAc/acetone, to give a crude oil (2.31 g). Trifluoroacetic anhydride (3.5 mL, 24.3 mmol) was added dropwise to a stirred solution of crude amine in pyridine (50 mL) at 5° C. The solution was stirred at 20° C. for 16 h. The solvent was evaporated and the residue purified by chromatography, eluting with a gradient (30-50%) of EtOAc/pet. ether, to give trifluoroacetamide 27 (0.51 g, 22%) as a yellow solid, mp (EtOAc/pet. ether) 89-90° C.; ¹H NMR δ 8.22-8.26 (m, 2H, H-8, NH), 7.71 (br dd, J=8.4, 7.0 Hz, 1H, H-6), 7.59 (d, J=8.4 Hz, 1H, H-5), 7.29 (br dd, J=8.5, 7.0 Hz, 1H, H-7), 5.45 (br s, 1H, NH), 4.12 (br dd, J=6.6, 6.5 Hz, 2H, CH₂N), 3.26-3.37 (m, 6H, 3×CH₂N), 1.84-1.95 (m, 2H, CH₂), 1.71-1.77 (m, 2H, CH₂), 1.48 [s, 9H, C(CH₃)₃]; ¹³C NMR δ 158.9, 157.3 (q, J=37 Hz), 156.8, 148.0, 135.6, 130.9, 126.5, 125.1, 120.4, 116 (q, J=288 Hz), 80.8, 44.5, 43.0, 38.8, 35.8, 29.7, 28.3 (3), 27.1; MS (FAB⁺) m/z 473 (MH⁺, 60%), 457 (10), 373 (100); HRMS (FAB⁺) calcd for C₂₀H₂₈F₃N₆O₄ (MH⁺) m/z 473.2124, found 473.2136. Anal. calcd for C₂₀H₂₇F₃N₆O₄: C, 50.8; H, 5.8; N, 17.8; found: C, 50.5; H, 5.7; N, 17.8%.

tert-Butyl 3-[(1,4-dioxido-1,2,4-benzotriazin-3-yl)amino]propyl{3-[(trifluoroacetyl)amino]propyl}carbamate (28). A solution of MCPBA (2.12 g, 8.6 mmol) in DCM (50 mL) was added dropwise to a stirred solution of 1-oxide 27 (3.13 g, 6.6 mmol) in DCM (250 mL) and NaHCO₃, (1.1 g, 13.2 mmol). The mixture was stirred at 20° C. for 16 h, partitioned between DCM (200 mL) and sat. aqueous KHCO₃ solution (100 mL). The organic fraction was dried and the solvent evaporated. The residue was purified by chromatography, eluting with a gradient (0-4%) of MeOH/40% EtOAc/DCM, to give (i) starting material 27 (2.04 g, 65%) and (ii) 1,4-dioxide 28 (252 mg, 8%) as a red solid, ¹H NMR δ 8.34 (d, J=8.7 Hz, 1H, H-8), 8.30 (d, J=8.4 Hz, 1H, H-5), 8.25 (br s, 1H, NH), 7.88 (br dd, J=8.4, 7.0 Hz, 1H, H-6), 7.52 (br dd, J=8.7, 7.0 Hz, 1H, H-7), 7.20 (br s, 1H, NH), 3.62 (dt, J=6.8, 6.7 Hz, 2H, CH₂N), 3.26-3.38 (m, 6H, 3×CH₂N), 1.92-1.98 (m, 2H, CH₂), 1.73-1.79 (m, 2H, CH₂), 1.49 [s, 9H, C(CH₃)₃]; ¹³C NMR δ 157.3 (q, J=37 Hz), 156.8, 149.8, 138.2, 135.9, 130.5, 127.4, 121.7, 117.4, 116.1 (q, J=288 Hz), 80.9, 44.4, 43.2, 38.9, 31.9, 29.7, 28.4 (3), 22.7; MS (FAB⁺) m/z 489 (MH⁺, 10%), 473 (12), 373 (15); HRMS (FAB⁺) calcd for C₂₀H₂₈F₃N₆O₅ (MH⁺) m/z 489.2073, found 489.2086.

tert-Butyl 3-aminopropyl{3-[(1,4-dioxido-1,2,4-benzotriazin-3-yl)amino]propyl}carbamate (29). A mixture of trifluoroacetamide 28 (541 mg, 1.11 mmol) and K₂CO₃ (0.77 g, 5.54 mmol) in MeOH (20 mL) and water (5 mL) was heated at reflux temperature for 1 h. The mixture was partitioned between CHCl₃ (50 mL) and water (30 mL). The aqueous fraction was extracted with CHCl₃ (3×30 mL), the combined organic fraction dried, and the solvent evaporated to give amine 29 (322 mg, 74%) as a red oil, ¹H NMR [(CD₃)₂SO] δ 10.50 (br s, 1H, NH), 8.21 (d, J=8.7 Hz, 1H, H-8), 8.13 (d, J=8.6 Hz, 1H, H-5), 7.94 (br dd, J=8.6, 7.5 Hz, 1H, H-6), 7.56 (br dd, J=8.6, 7.5 Hz, 1H, H-7), 7.94 (br s, 2H, NH₂), 3.39 (t, J=6.9 Hz, 2H, CH₂N), 3.11-3.21 (m, 6H, 3×CH₂N), 1.78-1.86 (m, 2H, CH₂), 1.49-1.58 (m, 2H, CH₂), 1.39 [s, 9H, C(CH₃)₃]; ¹³C NMR [(CD₃)₂SO] δ 154.7, 149.7, 138.1, 135.4, 129.8, 127.9, 121.0, 116.7, 78.3, 44.3, 43.9, 38.8, 38.4, 32.2, 31.6, 27.9 (3); MS (FAB⁺) m/z 393 (MH⁺, 15%), 377 (9), 338 (3); HRMS (FAB⁺) calcd for C₁₈H₂₉N₆O₄ (MH⁺) m/z 393.2250, found 393.2249.

N-[3-({3-[(1,4-Dioxido-1,2,4-benzotriazin-3-yl)amino]propyl}amino)propyl]-4-acridinecarboxamide (30). A solution of 4-acridinecarboxylic acid (846 mg, 4.35 mmol) and CDI (846 mg, 5.21 mmol) in DMF (20 mL) were stirred at 50° C. for 1 h The solvent was evaporated and the residue recrystallized from DCM/pet. ether to give 4-(1H-imidazol-1-ylcarbonyl)acridine (746 mg, 63%) which was used directly without characterization. A solution of the amine 29 (320 mg, 0.82 mmol) in DCM (10 mL) was added dropwise to a stirred solution of imidazolide (234 mg, 0.86 mmol) in THF (25 mL) at 5° C. and the solution was stirred at 20° C. for 16 h. The solvent was evaporated and the residue purified by chromatography, eluting with a gradient (0-5%) of MeOH/DCM, to give tert-butyl 3-[(4-acridinylcarbonyl)amino]propyl{3-[(1,4-dioxido-1,2,4-benzotriazin-3-yl)amino]propyl}carbamate (330 mg, 67%) as a red gum, ¹H NMR δ 11.92 (br s, 1H, CONH), 8.98 (dd, J=7.2, 1.5 Hz, 1H, H-3), 8.89 (s, 1H, H-9), 8.26-8.32 (m, 3H, H-5, H-5′, H-8′), 8.16 (d, J=8.3 Hz, 1H, H-1), 8.07 (d, J=8.8 Hz, 1H, H-8), 7.82-7.89 (m, 3H, H-3, H-6, H-6′), 7.65-7.69 (m, 1H, H-7′), 7.58-7.62 (m, 1H, H-7), 7.48 (br s, 1H, NH), 3.72 (dt, J=6.6, 6.0 Hz, 2H, CH₂N), 3.61 (dt, J=6.6, 6.4 Hz, 2H, CH₂N), 3.38-3.50 (m, 4H, 2×CH₂N), 2.04-0.08 (m, 2H, CH₂), 1.88-1.94 (m, 2H, CH₂), 1.40 [s, 9H, C(CH₃)₃]; MS (FAB⁺) m/z 598 (MH⁺, 8%), 582 (6); HRMS (FAB⁺) calcd for C₃₂H₃₆N₇O₅ (MH⁺) m/z 598.2778, found 598.2772.

HCl saturated MeOH (30 mL) was added to a solution of carbamate (328 mg, 0.55 mmol) in MeOH (30 mL) and the solution stirred at 20° C. for 16 h. The solution was evaporated and the residue dissolved in water (20 mL) the solution neutralized with KHCO₃ and extracted with CHCl₃ (5×50 mL). The combined organic fraction was dried and the solvent evaporated to give compound 30 (247 mg, 90%) as a red solid, ¹H NMR [(CD₃)₂SO] δ 11.38 (t, J=5.5 Hz, 1H, CONH, 10.50 (br s, 1H, NH), 9.28 (s, 1H, H-9), 8.71 (dd, J=7.1, 1.5 Hz, 1H, H-3), 8.35 (dd, J=8.4, 1.5 Hz, 1H, H-1), 8.24 (d, J=8.7 Hz, 1H, H-5), 8.19 (d, J=8.3 Hz, 1H, H-8), 8.14 (d, J=8.5 Hz, 1H, H-8′), 8.03 (d, J=8.5 Hz, 1H, H-5′), 7.92-7.96 (m, 1H, H-6), 7.83-7.88 (m, 1H, H-6′), 7.75 (dd, J=8.3, 7.1 Hz, 1H, H-2), 7.65-7.68 (m, 1H, H-7), 7.48-7.54 (m, 1H, H-7′), 7.38 (s, 1H, NH), 3.64 (dt, J=6.9, 5.9 Hz, 2H, CH₂N), 3.46 (t, J=6.7 Hz, 2H, CH₂N), 2.79 (t, J=6.9 Hz, 2H, CH₂N), 2.70 (t, J=6.5 Hz, 2H, CH₂N), 1.88-1.94 (m, 2H, CH₂), 1.76-1.82 (m, 2H, CH₂); ¹³C NMR [(CD₃)₂SO] δ 164.7, 149.6, 147.0, 145.4, 138.5, 138.0, 135.3, 134.4, 132.6, 131.8, 129.7, 128.5, 128.4, 128.3, 126.7, 1264.4, 126.3, 125.5, 125.2, 121.0, 116.7, 47.1, 46.9, 39.6, 37.2, 29.3, 28.2; MS (FAB⁺) m/z 498 (MH⁺, 15%), 482 (5); HRMS (FAB⁺) calcd for C₂₇H₂₈N₇O₃ (MH⁺) m/z 498.2254, found 498.2258. Anal. calcd for C₂₇H₂₇N₇O₃.2H₂O: C, 60.8; H, 5.9; N, 18.4; found: C, 60.7; H, 5.6; N, 17.1%.

### Example K

N-[3-({3-[(1,4-dioxido-1,2,4-benzotriazin-3-yl)amino]propyl}amino)propyl]-1-phenazinecarboxamide hydrochloride (31). A solution of the amine 29 (223 mg, 0.57 mmol) in THF (10 mL) was added dropwise to a stirred solution of 1-(1H-imidazol-1-ylcarbonyl)phenazine (171 mg, 0.63 mmol) in THF (25 mL) at 5° C. and the solution was stirred at 20° C. for 16 h. The solvent was evaporated and the residue purified by chromatography, eluting with a gradient (0-3%) of MeOH/DCM, to give tert-butyl 3-[(1-phenazinecarbonyl)amino]propyl{3-[(1,4-dioxido-1,2,4-benzotriazin-3-yl)amino]propyl}carbamate (137 mg, 40%) as a red gum, ¹H NMR δ 11.22 and 11.06 (2×s, 1H, CONH, 9.03 (dd, J=7.1, 1.4 Hz, 1H, H-2), 8.64 and 8.27 (2×s, 1H, NH), 8.42 (d, J=8.2 Hz, 1H, H-9), 8.29-8.37 (m, 3H, H-4, H-6, H-8′″), 7.86-8.03 (m, 5H, H-3, H-7, H-8, H-5′″, H-6′″), 7.49-7.56 (m, 1H, H-7′″), 3.69-3.77 (m, 2H, CH₂N), 3.63-3.68 (m, 2H, CH₂N), 1.93-2.10 (m, 4H, 2×CH₂N), 1.67 and 1.63 [2×s, 9H, C(CH₃)₃], 1.45-1.53 (m, 4H, 2×CH₂); MS (FAB⁺) m/z 599 (MH⁺, 12%), 583 (3); HRMS (FAB⁺) calcd for C₃₁H₃₅N₈O₅ (MH⁺) m/z 599.2730, found 599.2733. HCl saturated MeOH (5 mL) was added to a solution of carbamate (135 mg, 0.23 mmol) in MeOH (20 mL) and the solution stirred at 20° C. for 16 h. The solution was evaporated and the residue dissolved in water (20 mL) the solution neutralized with dil. aqueous NH₃ and extracted with CHCl₃ (5×50 mL). The combined organic fraction was dried and the solvent evaporated to give compound 31 (97 mg, 85%) as a red solid, which was converted to the HCl salt and recrystallized, mp (MeOH/EtOAc) 163-169° C.; ¹H NMR [(CD₃)₂SO] δ 10.29 (t, J=5.8 Hz, 1H, CONH), 9.26 (br s, 2H, NH₂⁺Cl⁻), 8.97 (t, J=6.1 Hz, 1H, NH), 8.59 (dd, J=9.0, 2.0 Hz, 1H, H-2), 8.55 (dd, J=9.0, 2.0 Hz, 1H, H-9), 8.41 (dd, J=8.7, 1.3 Hz, 1H, H-4), 8.28 (dd, J=7.9, 2.0 Hz, 1H, H-6), 8.19 (d, J=8.2 Hz, 1H, H-8′″), 7.98-8.08 (m, 5H, H-3, H-7, H-8, H-5′″, H-6′″), 7.60 (ddd, J=8.7, 7.1, 1.4 Hz, 1H, H-7′″), 3.65-3.69 (m, 2H, H′), 3.55-3.59 (m, 2H, H-3″), 3.04-3.13 (m, 4H, H-3′, H-1″), 2.03-2.15 (m, 4H, H-2′, H-2″); ¹³C NMR [(CD₃)₂SO] δ 164.8, 149.8, 142.6, 142.5, 141.3, 139.9, 137.5, 136.5, 133.4, 132.6, 131.8, 131.6, 131.0, 130.5, 130.2, 129.5, 129.0, 127.5, 121.8, 116.2, 44.6, 44.2, 38.1, 36.4, 25.9, 25.1. Anal. calcd for C₂₆H₂₇ClN₈O₃.MeOH: C, 57.2; H, 5.5; N, 19.8; found: C, 57.3; H, 5.8; N, 20.0%.

### Example L

N-[3-({3-[(1,4-dioxido-1,2,4-benzotriazin-3-yl)amino]propyl}amino)propyl]-9-methyl-1-phenazinecarboxamide hydrochloride (32). A solution of the amine 29 (265 mg, 0.68 mmol) in THE (10 mL) was added dropwise to a stirred solution of 1-(1H-imidazol-1-ylcarbonyl)-9-methylphenazine (214 mg, 0.74 mmol) in THF (25 mL) at 5° C. and the solution was stirred at 20° C. for 16 h. The solvent was evaporated and the residue purified by chromatography, eluting with a gradient (5-10%) of MeOH/DCM, to give tert-butyl 3-[(1,4-dioxido-1,2,4-benzotriazin-3-yl)amino]propyl(3-{[(9-methyl-1-phenazinyl)carbonyl]amino}propyl)carbamate (168 mg, 40%) as a red gum, MS (FAB⁺) m/z 613 (MH⁺, 20%), 597 (5), 513 (15), 497 (5); HRMS (FAB⁺) calcd for C32H37N₈O₅ (MH⁺) m/z 613.2887, found 613.2881. HCl saturated MeOH (5 mL) was added to a solution of carbamate (168 mg, 0.27 mmol) in MeOH (20 mL) and the solution stirred at 20° C. for 16 h. The solution was evaporated and The residue dissolved in water (20 mL) the solution neutralized with dil. aqueous NH₃ and extracted with CHCl₃ (5×50 mL). The combined organic fraction was dried and the solvent evaporated to give compound 32 (121 mg, 86%) as a red solid, which was converted to the HCl salt and recrystallized, mp (MeOH/EtOAc) 183-186° C.; ¹NMR [(CD₃)₂SO] δ 10.45 (t, J=5.8 Hz, 1H, CONH), 9.18 (br s, 2H, NH₂⁺C⁻), 8.73 (t, J=6.2 Hz, 1H, NH), 8.63 (dd, J=7.0, 1.4 Hz, 1H, H-2), 8.39 (dd, J=8.7, 1.4 Hz, 1H, H-4), 8.18 (d, J=8.7 Hz, 1H, H-8′″), 8.03-8.11 (m, 3H, H-3, H-5′″, H-7), 7.92 (ddd, J=8.5, 7.1, 1.3 Hz, 1H, H-6′″), 7.87-7.93 (m, 2H, H-6, H-8), 7.57 (ddd, J=8.7, 7.1, 1.3 Hz, 1H, H-7′″), 3.61-3.66 (m, 2H, H′), 3.51-3.57 (m, 2H, H-3″), 2.98-3.10 (m, 4H, H-3′, H-1″), 2.86 (s, 3H, CH₃), 2.08-2.15 (m, 2H, H-2′), 1.99-2.05 (m, 2H, H-2″); ¹³C NMR [(CD₃)₂SO] δ 164.5, 149.7, 142.6, 142.3, 140.4, 138.7, 137.7, 136.6, 136.0, 133.7, 132.7, 131.5, 131.2, 130.2, 130.1, 130.0, 127.1, 127.0, 121.0, 116.4, 44.8, 44.7, 37.9, 36.6, 26.2, 25.1, 17.5; MS (FAB⁺) m/z 513 (MH⁺, 20%), 497 (5); HRMS (FAB⁺) calcd for C₂₇H₂₉N₈O₃ (MH⁺) m/z 513.2363, found 513.2352.

### Example M

### N-[2-({2-[(1,4-Dioxido-1,2,4-benzotriazin-3-yl)amino]ethyl}amino)ethyl]-4-acridinecarboxamide (37)

tert-Butyl bis-(2-aminoethyl)carbamate (33). Diethylenetriamine (9.9 mL, 96 mmol) was added to a solution of CF₃CO₂Et (22.8 mL, 192 mmol) in dry ether (80 mL) at 5° C. and the reaction mixture was stirred at 20° C. for 20 h. The resulting white precipitate was filtered and washed with cold ether (100 mL), dried under vacuum to give 2,2,2-trifluoro-N-[2-({2-[(trifluoroacetyl)amino]ethyl}amino)ethyl]acetamide (17.26 g, 61%), ¹NMR [(CD₃)₂SO] δ 7.26 (br, 2H, 2×CONH), 3.43 (br s, 4H, 2×CH₂), 2.86 (t, J=5.8 Hz, 4H, 2×CH₂); ¹³C NMR [(CD₃)₂SO] δ 157.7 (q, J=37 Hz), 115.8 (q, J=288 Hz), 47.3 (2), 39.3 (2).

Di-tert butyldicarbonate (8.26 g, 37.8 mmol) was added to a solution of acetamide (10.15 g, 34.4 mmol) in THF (100 mL) at 0° C. and the mixture was stirred at 20° C. for 20 h. Saturated aqueous N₄Cl (80 mL) added and the mixture stirred at 20° C. for 5 h. The mixture was extracted with DCM (3×50 mL), dried, and the solvent evaporated to give tert-butyl bis{2-[(trifluoroacetyl)amino]ethyl}carbamate (13.5 g, 100%), ¹H NMR [(CD₃)₂SO] δ 9.47 (br, 1H, CONH), 9.40 (br, 1H, CONH), 3.30 (m, 8H, 4×CH₂), 1.38 [s, 9H, C(CH₃)₃]; ¹³C NMR [(CD₃)₂SO] δ 156.4 (q, J=36 Hz), 154.7, 115.8 (q, J=288 Hz), 78.9, 45.4, 45.0, 37.7, 37.4, 27.7 (3). Conc. ammonia (50 mL) was added to a solution of carbamate (14.0 g, 35.5 mmol) in MeOH (100 mL) and heated at reflux temperature for 20 hr. The solvent was evaporated to give diamine 33 as a yellow Loam, ¹H NMR [(CD₃)₂SO] δ 3.39 (t, J=6.4 Hz, 4H, 2×CH₂), 2.94 (t, J=6.4 Hz, 4H, 2×CH₂), 1.42 [s, 9H, C(CH₃)₃]; ¹³C NMR [(CD₃)₂SO] δ 154.9, 79.9, 45.1 (2), 37.4 (2), 27.9 (3).

Di-tert-butyl 2-aminoethyl{2-[(1-oxido-1,2,4-benzotriazin-3-yl)amino]ethyl}dicarbamate (35). A solution of chloride 3 (1.0 g, 5.5 mmol), diamine 33 (4.47 g, 22.0 mmol), and Et₃N (2.24 g, 22 mmol) in DME (20 mL) was heated at 90° C. for 3 h. The solvent was evaporated and the residue purified by chromatography, eluting with a gradient (0-4%) of MeOH/DCM to give (i) tert-butyl bis{2-[(1-oxido-1,2,4-benzotriazin-3-yl)amino]ethyl}carbamate (0.34 g, 25%), ¹H NMR δ 8.17 (br d, J=8.5 Hz, 2H, H-8), 7.71-7.62 (m, 2H, H-6), 7.52 (br d, J=8.3 Hz, 2H, H-8), 7.26-7.22 (m, 2H, H-7), 6.15 (br s, 1H, NH), 5.95 (br s, 1H, NH), 3.71 (br q, J=5.8 Hz, 4H, 2×CH₂), 3.37 (br s, 4H, 2×CH₂), 1.50 [s, 9H, C(CH₃)₃]; ¹³C NMR δ 158.9, 156.3 (2), 148.6 (2), 135.5 (2), 130.9 (2), 126.4 (2), 124.9 (2), 120.3 (2), 80.7, 47.7 (2), 40.9 (2), 28.4 (3); and (ii) crude tert-butyl 2-aminoethyl{2-[(1-oxido-1,2,4-benzotriazin-3-yl)amino]ethyl}carbamate 34 (1.38 g, 72%) as a yellow foam.

Di-tert-butyldicarbonate (2.7 g, 12.4 mmol) was added to a solution of carbamate 34 (1.38 g, 4.0 mmol) in THF (50 mL) and the solution stirred at 20° C. for 36 h. Water (100 mL) was added and the mixture stirred at 20° C. for 1 h. The mixture was extracted with DCM (3×50 mL), the organic fraction dried, and the solvent evaporated. The residue was purified by chromatography, eluting with a gradient (0-1%) of aqueous NH₃/(0-7%) MeOH/DCM, to give carbamate 35 (0.94 g, 52%) as a yellow powder, mp (DCM/hexane) 160-163° C.; ¹H NMR [(CD₃)₂SO] δ 8.14 (dd, J=8.3, 0.7 Hz, 1H, H-8), 8.00 and 7.92 (2×br s, 1H, CONH), 7.79 (dd, J=7.5, 1.2 Hz, 1H, H-6), 7.58 (br d, J=7.5 Hz, 1H, H-5), 7.34 (dd, J=7.7, 1.2 Hz, 1H, H-7), 6.81 (br s, 1H, NH), 3.43-3.47 (m, 2H, CH₂), 3.37 (m, 2H, CH₂), 3.22 (t, J=6.2 Hz, 2H, CH₂), 3.03-3.07 (m, 2H, CH₂), 1.34 [s, 9H, C(CH₃)₃], 1.34 and 1.27 [2×s, 9H, C(CH₃)₃]; ¹³C NMR [(CD₃)₂SO] δ 158.9, 155.5, 154.7, 148.2, 135.7, 130.1, 126.0, 124.6, 119.8, 78.4, 77.5, 47.0, 46.2, 38.5, 38.1, 28.1(3), 27.8 (3). Anal. calcd for C₂₁H₃₂N₆O₅C, 56.2; H, 7.2; N, 18.7; found C, 56.5; H, 7.5; N, 18.8%.

Di-tert-butyl 2-aminoethyl{2-[(1,4-dioxido-1,2,4-benzotriazin-3-yl)amino]ethyl}dicarbamate (36). MCPBA (247 mg, 1.0 mmol) was added to a solution of 1-oxide 35 (300 mg, 0.67 mmol) in DCM (10 mL) and the mixture was stirred at 20° C. for 16 h. The mixture was partitioned between dil. aqueous NH₃ (50 mL) and DCM (50 mL) and the aqueous fraction extracted with DCM (3×30 mL). The combined organic fraction was dried, and the solvent evaporated. The residue was purified by chromatography, eluting with a gradient (0-2%) of MeOH/DCM, to give (i) starting material (188 mg, 62%) and (ii) 1,4-dioxide 36 (122 mg 39%), mp (DCM/hexane) 128-134° C.; ¹H NMR [(CD₃)₂SO] δ 8.39 and 8.32 (2×br s, 1H, CONH), 8.21 (dd, J=8.7, 0.7 Hz, 1H, H-8), 8.14 (t, J=8.0 Hz, 1H, H-5), 7.94 (t, J=7.6 Hz, 1H, H-6), 7.57 (t, J=7.9 Hz, 1H, H-7), 6.77 (br s, 1H, NH), 3.50-3.54 (m, 2H, CH₂), 3.41-3.44 (m, 2H, CH₂), 3.20 (t, J=6.5 Hz, 2H, CH₂), 3.03 (br q, J=5.6 Hz, 2H, CH₂), 1.33 [s, 9H, C(CH₃)₃], 1.33 and 1.26 [2×s, 9H, C(CH₃)₃]; ¹³C NMR [(CD₃)₂SO] δ 155.5 154.6, 149.8, 138.1, 135.5, 129.9, 127.0, 121.0, 116.81, 78.6, 77.4, 46.8, 46.1, 38.5, 38.0, 28.8, 27.7; HRMS calcd for C₂₁H₃₃H₆O₆ (M⁺) m/z 465.2462, found 465.2456.

N-[2-({2-[(1,4-Dioxido-1,2,4-benzotriazin-3-yl)amino]ethyl}amino)ethyl]-4-acridinecarboxamide (37). A solution of carbamate 36 (252 mg, 0.54 mmol) in HCl saturated MeOH (10 mL) was stirred at 20° C. for 24 h. The solvent was evaporated and the residue partitioned between aqueous NH₃ (20 mL) and DCM (50 mL). The aqueous fraction was extracted with DCM (5×20 mL) and the combined organic extracts dried. The solvent was evaporated to give N¹-(2-aminoethyl)-N²-(1,4-dioxido-1,2,4-benzotriazin-3-yl)-1,2-ethanediamine (109 mg, 76%), ¹H NMR δ 8.33 (d, J=8.7 Hz, 1H, H-8), 8.30 (d, J=8.8 Hz, 1H, H-5), 7.87 (ddd, J=8.5, 7.1, 1.0 Hz, 1H, H-6), 7.50 (ddd, J=8.4, 7.1, 1.2 Hz, 1H, H-7), 3.70 (t, J=5.9 Hz, 2H, CH₂), 2.98 (t, J=5.9H, 2H, CH₂), 2.84 (t, J=5.6 Hz, 2H, CH₂), 2.74 (t, J=5.6 Hz, 2H, CH₂), 2×NH and NH₂ not observed.

A solution of N¹-(2-aminoethyl)-N²-(1,4-dioxido-1,2,4-benzotriazin-3-yl)-1,2-ethanediamine (96 mg, 0.36 mmol) and 4-(1H-imidazol-1-ylcarbonyl)acridine (119 mg, 0.43 mmol) in DMF (5 mL) was stirred at 20° C. for 5 h. The solvent was evaporated and the residue purified by chromatography, eluting with a gradient (0-2%) of aqueous NH₃/(0-7%) MeOH/DCM, to give compound 37 (168 mg, 99%) as a red solid, rap (DCM/hexane) 151-154° C.; ¹H NMR δ 11.98 (t, J=5.3 Hz, 1H, CONH), 8.82 (dd, J=8.2, 1.4 Hz, 1H, ArH), 8.81 (s, 1H, ArH), 8.08-8.17 (m, 4H, 4×ArH), 7.97 (d, J=8.3 Hz, 1H, ArH), 7.75-7.82 (m, 2H, 2×ArH), 7.60 (dd, J=8.2, 7.2 Hz, 1H, ArH), 7.54 (ddd, J=8.3, 7.3, 0.9 Hz, 1H, ArH), 7.40 (ddd, J=8.6, 7.2, 1.2 Hz, 1H, ArH), 3.86 (br q, J=5.8 Hz, 2H, CH₂), 3.70 (t, J=5.8 Hz, 2H, CH₂), 3.17 (br q, J=6.3 Hz, 4H, 2×CH₂), 2×NH not observed; ¹³C NMR δ 166.5, 149.7, 147.4, 146.2, 138.0, 137.7, 135.6, 135.3, 132.5, 131.4, 130.2, 128.9, 128.0, 126.9 (2), 126.7, 126.3, 125.9, 125.4, 121.5, 117.2, 48.6, 47.8, 40.6, 39.3; HRMS (FAB⁺) calcd for C₂₅H₂₄N₇O₃ (MH⁺) m/z 470.1941 found 470.1934. Anal. calcd for C₂₅H₂₃N₇O₃.1½H₂O: C, 60.5; H, 5.3; N, 19.8; found C, 60.5; H, 5.0; N, 20.0%.

### Example N

### N-{3-[{3-[(1,4-Dioxido-1,2,4-benzotriazin-3-yl)amino]propyl}(methyl)amino]propyl}-4-acridinecarboxamide (41)

2,2,2-Trifluoro-N-[3-(methyl{3-[(1-oxido-1,2,4-benzotriazin-3-yl)amino]propyl}amino)propyl]acetamide (38). A solution of chloride 3 (2.07 g, 11.4 mmol), N¹-(3-aminopropyl)-N¹-methyl-1,3-propanediamine (3.31 g, 22.8 mmol) and Et₃N (3.2 mL, 22.8 mmol) in DCM (200 mL) was stirred at 20° C. for 2 d. The solvent was evaporated and the residue dissolved in MeCN (150 mL). Ethyl trifluoroacetate (5.4 mL, 45.6 mmol) and water (0.8 mL, 45.6 mmol) added and the solution heated at reflux temperature for 16 h. The solvent was evaporated, and the residue purified by chromatography, eluting with a gradient (0-1%) of Et₃N/(0-10%) MeOH/DCM, followed by further chromatography, eluting with 10% MeOH/DCM, to give 1-oxide 38 (1.89 g, 43%) as a yellow solid, mp (DCM) 111-115° C.; ¹H NMR δ 9.04 (br s, 1H, NH), 8.25 (dd, J=8.7, 1.4 Hz, 1H, H 8′), 7.70 (ddd, J=8.4, 7.1, 1.4 Hz, 1H, H-6′), 7.57 (d, J=8.4 Hz, 1H, H-5′), 7.29 (ddd, J=8.7, 7.1, 1.1 Hz, 1H, H-7′), 6.17 (br s, 1H, NH), 3.58 (dd, J=6.6, 5.8 Hz, 2H, CH₂N), 3.49 (br t, J=6.0 Hz, 2H, CH₂N), 2.52-2.58 (m, 4H, 2×CH₂N), 2.27 (s, 3H, NCH₃), 1.84-1.90 (m, 2H, CH₂), 1.75-1.82 (m, 2H, CH₂); ¹³C NMR δ 158.9, 157.3 (q, J=36 Hz), 148.8, 135.6, 130.8, 126.4, 124.9, 120.4, 116.1 (q, J=288 Hz), 57.1, 56.4, 41.3, 40.3 (2), 26.3, 24.4; MS (FAB⁺) m/z 387 (MH⁺, 100%), 371 (8), 338 (30); HRMS (FAB⁺) calcd for C₁₆H₂₂F₃N₆O₂ (MH⁺) m/z 387.1756, found 387.1765. Anal. calcd for C₁₆H₂₁F₃N₆O₂.½MeOH: C, 49.2; H, 5.8; N, 20.9; found: C, 49.1; H, 5.5; N, 20.7%.

N-{3-[{3-[(1,4-Dioxido-1,2,4-benzotriazin-3-yl)amino]propyl}(methyl)amino]propyl}-2,2,2-trifluoroacetamide (39). Trifluoroacetic anhydride (4.31 mL, 29.2 mmol) was added to a stirred solution of 1-oxide 38 (1.13 g, 2.92 mmol) in CHCl₃ (50 mL) and the solution stirred at 20° C. for 30 min. The solution was cooled to −10° C. and 70% H₂O₂ (2 mL) (CAUTION) added dropwise. The solution was stirred at 20° C. for 30 d, partitioned between CHCl₃ (50 mL) and sat aqueous KHCO₃ (50 mL). The aqueous fraction was extracted with CHCl₃ (3×30 mL), the combined organic fraction dried and the solvent evaporated (CAUTION: safety shield). The residue was purified by chromatography, eluting with 10% MeOH/DCM, to give (i) starting material 38 (275 mg, 24%) and (ii) 1,4-dioxide 39 (319 mg, 27%) as a red gum, ¹H NMR [(CD₃)₂SO] δ 9.44 (br s, 1H, NH), 8.45 (t, J=5.9 Hz, 1H, NH), 8.20 (d, J=8.8 Hz, 1H, H-8′), 8.12 (d, J=8.6 Hz, 1H, H-5′), 7.93 (ddd, J=8.6, 7.1, 1.2 Hz, 1H, H-6′), 7.57 (ddd, J=8.8, 7.1, 1.3 Hz, 1H, H-7′), 3.42-3.47 (ma, 2H, CH₂N), 3.21-3.25 (m, 2H, CH₂N), 2.39 (t, J=6.7 Hz, 2H, CH₂N), 2.32 (t, J=6.9 Hz, 2H, CH₂N), 2.16 (s, 3H, NCH₃), 1.72-1.80 (m, 2H, CH₂), 1.61-1.68 (m, 2H, CH₂); ¹³C NMR [(CD₃)₂SO] δ 155.9 (q, J=36 Hz), 149.7, 138.1, 135.4, 129.8, 126.7, 121.0, 116.7, 115.9 (q, J=288 Hz), 54.9, 54.6, 41.4, 39.5, 37.6, 25.9, 25.8; MS (FAB⁺) m/z 403 (MH⁺, 25%), 387 (5); HRMS (FAB⁺) calcd for C₁₆H₂₂F₃N₆O₃ (MH⁺) m/z 403.1706, found 403.1695.

N-{3-[{3-[(1,4-Dioxido-1,2,4-benzotriazin-3-yl)amino]propyl}(methyl)amino]propyl}-4-acridinecarboxamide (41). A solution of trifluoroacetamide 39 (175 mg, 0.44 mmol) and NH₄OH (5 mL) in MeOH (20 mL) was stirred at 30° C. for 4 h. The solvent was evaporated and the residue dried to give N¹-(3-aminopropyl)-N³-(1,4-dioxido-1,2,4-benzotriazin-3-yl)-N¹-methyl-1,3-propanediamine (40) as a red gum, ¹H NMR [(CD₃)₂SO] δ 8.43 (br s, 1H, NH) 8.21 (d, J=8.5 Hz, 1H, H-8′), 8.13 (d, J=8.4 Hz, 1H, H-5′), 7.94 (ddd, J=8.4, 7.1, 1.2 Hz, 1H, H-6′), 7.75 (br s, 2H, NH₂), 7.57 (ddd, J=8.7, 7.2, 1.3 Hz, 1H, H-7′), 3.45 (t, J=6.8 Hz, 2H, CH₂N), 3.20-3.25 (m, 2H, CH₂N), 2.88 (dd, J=7.4, 7.2 Hz, 2H, CH₂N), 2.40-2.46 (m, 2H, CH₂N), 2.20 (s, 3H, NCH₃), 1.77-1.83 (m, 2H, CH₂), 1.68-1.75 (m, 2H, CH₂); MS (FAB⁺) m/z 307 (MH⁺, 2%), 291 (5); HRMS (FAB⁺) calcd for C₁₄H₂₃N₆O₃ (MH⁺) m/z 307.1883, found 307.1883. The amine 40 was dissolved in DCM (5 mL) and added to a stirred solution of 4-(1H-imidazol-1-ylcarbonyl)acridine (125 mg, 0.46 mmol) in THF (20 mL) and the solution stirred at 20° C. for 16 h. The solvent was evaporated and the residue purified by chromatography, eluting with a gradient (0-1%) of Et₃N/(0-15%) MeOH/DCM, to give compound 41 (146 mg, 66%) as a red solid, mp (EtOAc/DCM) 169-171° C.; ¹H NMR [(CD₃)₂SO] δ 11.41 (t, J=5.3 Hz, 1H, CONH), 9.31 (s, 1H, H-9), 8.69 (dd, J=7.0, 14 Hz, 1H, H-3), 8.43 (t, J=5.6 Hz, 1H, NH), 8.38 (d, J=7.4 Hz, 1H, H-1), 8.32 (d, J=8.8 Hz, 1H, H-5), 8.21 (d, J=8.4 Hz, 1H, H-8), 8.16 (d, J=8.7 Hz, 1H, H-8′), 8.09 (d, J=8.7 Hz, 1H, H-5′), 7.96 (ddd, J=8.7, 7.1, 1.1 Hz, 1H, H-6′), 7.91 (dd, J=8.8, 7.5 Hz, 1H, H-6), 7.74 (dd, J=7.4, 7.0 Hz, 1H, H-2), 7.69 (br dd, J=8.7, 7.1 Hz, 1H, H-7′), 7.55 (dd, J=8.4, 7.5 Hz, 1H, H-7), 3.60-3.65 (m, 2H, CH₂N), 3.42-3.48 (m, 2H, CH₂N), 3.39 (s, 3H, NCH₃), 3.00-3.08 (m, 2H, CH₂N), 2.60-2.68 (m, 2H, CH₂N), 2.02-2.08 (m, 2H, CH₂), 1.92-1.98 (m, 2H, CH₂); MS (FAB⁺) m/z 512 (MH⁺, 25%), 496 (10); HRMS (FAB⁺) calcd for C₂₈H₃₀N₇O₃ (MH⁺) m/z 512.2410, found 512.2424.

### Example O

N-{3-[{3-[(1,4-Dioxido-1,2,4-benzotriazin-3-yl)amino]propyl}(methyl)amino]propyl}-8-quinolinecarboxamide (42). A solution of 8-quinolinecarboxylic acid (90 mg, 0.5 mmol) and CDI (97 mg, 0.6=mol) in DMF (5 mL) was stirred at 55° C. for 24 h. The solution was diluted with dry benzene (10 mL), Sephadex LH-20 (300 ma) was added and the mixture stirred at 20° C. for 1 h. The mixture was filtered and the solvent evaporated. The residue was dissolved in dry THF (5 mL) and a solution of (40) (80 mg, 0.25 mmol) in THF (5 mL) added, and the solution stirred at 20° C. for 70 h. The solvent was evaporated and the residue purified by chromatography, eluting with a gradient (0-2%) of aqueous NH₃/(0-8%) MeOH/DCM, to give compound 42 (110 mg, 91%) as a red powder, mp (DCM/pet. ether) 119-121° C.; ¹H NMR δ 11.39 (br s, 1H, CONH), 8.96 (dd, J=4.3, 1.8 Hz, 1H, ArH), 8.74 (dd, J=7.3, 1.5 Hz, 1H, ArH), 8.34 (dd, J=8.8, 1.8 Hz, 1H, ArH), 8.25 (d, J=8.3 Hz, 1H, ArH), 8.17 (d. J=8.6 Hz, 1H, ArH), 7.98 (br s, 1H, NH), 7.92 (dd, J=8.1, 1.5 Hz, 1H, ArH), 7.78 (dd, J=8.1, 1.1 Hz, 1H, ArH), 7.62 (t, J=7.7 Hz, 1H, ArH), 7.48 (dd, J=8.3, 1H, 4.0 Hz), 7.43 (dd, J=7.9, 1.0 Hz, 1H, ArH), 3.68-3.73 (m, 4H, 2×CH₂), 3.05 (br m, 4H, 2×CH₂), 2.67 (s, 3H, CH₃), 2.25-2.17 (m, 4H, 2×CH₂); ¹³C NMR δ 166.4, 149.7, 149.6, 145.4, 138.2, 137.7, 135.6, 133.6, 132.0, 130.3, 128.5, 128.4, 127.1, 126.4, 121.5, 121.0, 117.3, 54.7, 54.5, 40.6, 39.4, 37.2, 25.5, 24.5; MS (FAB⁺) m/z 462 (MH⁺, 25%), 446 (5); JMS calcd for C₂₄H₂₈N₇O₃ (MH⁺) m/z 462.2254, found 462.2249. Anal. calcd for C₂₄H₂₇N₇O₃: C, 62.5; H, 5.9; N, 21.2; found: C, 62.1; H, 6.0; N, 21.2%.

### Example P

N-{3-[{3-[(1,4-dioxido-1,2,4-benzotriazin-3-yl)amino]propyl}(methyl)amino]-propyl}-2-(4-pyridyl)-8-quinolinecarboxamide (43). A solution of 2-(4-pyridyl)-quinoline-8-carboxylic acid (160 mg, 0.62 mmol) and CDI (150 mg, 0.92 mmol) in DMF (10 mL) was stirred at 55° C. for 24 h. The solution was cooled to 20° C., diluted with dry benzene (15 mL), Sephadex LH-20 (300 ma) was added and the mixture stirred at 20° C. for 1 h. The mixture was filtered and the solvent evaporated. The residue was dissolved in dry THF (5 mL) and a solution of (39) (90 mar, 0.33 mmol) in THF (5 mL) added, and the solution stirred at 20° C. for 4 days. The solvent was evaporated and the residue purified by chromatography, eluting with a gradient (0-2%) of aqueous NH₃/(0-8%) MeOH/DCM, to give compound 43 (160 mg, 94%) as a red powder, mp (DCM/pet. ether) 179-181° C.; ¹H NMR δ 11.08 (br s, 1H, CONH), 8.86 (dd, J=4.5, 1.6 Hz, 2H, ArH), 8.78 (dd, J=7.4, 1.5 Hz, 1H, ArH), 8.37 (d, J=8.6 Hz, 1H, ArH), 8.21 (d, J=8.6 Hz, 1H, ArH), 8.10 (d, J=8.6 Hz, 1H, ArH), 7.95 (dd, J=8.2, 1.4 Hz, 1H, ArH) 7.92-7.90 (m, 4H, NH, 3×ArH), 7.98 (ddd, J=8.6, 7.5, 1.3 Hz, 1H, ArH), 7.66 (t, J=7.7 Hz, 1H, ArH) 7.40 (ddd, J=8.6, 7.2, 1.2 Hz, 1H, ArH), 3.74 (br q, J=6.4 Hz, 2H, CH₂), 3.61 (br m, H, CH₂), 2.85 (br m, 2H, CH₂), 2.81 (br m, 2H, CH₂), 2.45 (s, 3H, CH₃), 2.17 (br q, J=7.2 Hz, 2H, CH₂), 1.98 (br m, 2H, CH₂); ¹³C NMR δ 166.1, 154.5, 150.9, 149.7, 146.2, 145.3, 139.0, 138.2, 135.5, 134.4, 131.5, 130.2, 129.4, 127.9, 127.2, 126.9, 121.7, 121.5, 118.7, 117.2, 55.3, 55.2, 41.0, 40.1, 37.7, 26.6, 24.7; HRMS (FAB⁺) calcd for C₂₉H₃₁N₈O₃ (MH⁺) m/z 539.2519, found 539.2527. Anal. calcd for C₂₉H₃₀N₈O₃.½H₂O: C, 64.7; H, 5.6; N, 20.8; found: C, 64.1; H, 5.7; N, 20.6%.

### Example Q

N-{3-[{3-[(1,4-dioxido-1,2,4-benzotriazin-3-yl)amino]propyl}(methyl)amino]-propyl}-5-methyl-4-acridinecarboxamide (44). A solution of 5-methylacridine-4-carboxylic acid (0.13 g, 0.55 mmol) and CDI (0.21 g, 1.3 mmol) in DMF (5 mL) was stirred at 55° C. for 24 h. The solution was diluted with dry benzene (10 mL), Sephadex LH-20 (300 mg) was added and the mixture stirred at 20° C. for 1 h. The mixture was filtered and the solvent evaporated. The residue was dissolved in dry THF (5 mL) and a solution of 40 (80 mg, 0.27 mmol) in THF (5 mL) added, and the solution stirred at 20° C. for 70 h. The solvent was evaporated and the residue purified by chromatography, eluting with a gradient (0-2%) of aqueous NH₃/(0-8%) MeOH/DCM, to give compound 44 (0.13 g, 88%) as a red powder, mp (DCM/pet. ether) 158-162° C.; ¹H NMR δ 12.08 (br s, 1H, CONH), 8.83 (d, J=6.9 Hz, 1H, ArH), 8.76 (s, 1H, NH), 8.06 (t, J=8.9 Hz, 2H, ArH), 7.97 (br d, J=8.4 Hz, 2H, ArH), 7.83 (d, J=8.4 Hz, 1H, ArH), 7.66 (d, J=6.7 Hz, 1H, ArH), 7.56-7.63 (m, 2H, ArH), 7.46 (dd, J=7.6, 6.5 Hz, 1H, ArH), 7.30 (d, J=7.9 Hz, 1H, ArH), 3.77 (br q, J=6.3 Hz, 2H, CH₂), 4.83 (br m, 2H, CH₂), 3.08 (br m, 4H, 2×CH₂), 2.83 (s, 3H, CH₃), 2.67 (br s, 3H, CH₃), 2.31 (br m, 2H, CH₂), 2.15 (br m, 2H, CH₂); ¹³C NMR δ 166.5, 149.6, 146.9, 145.1, 137.9, 137.9, 135.8, 135.3, 135.1, 132.4, 131.2, 130.0, 127.9, 126.8, 126.4, 126.3, 126.2, 125.8, 125.2, 121.3, 117.0, 55.1, 54.5, 40.5, 39.2, 37.4, 26.1, 24.5, 19.0; HRMS (FAB⁺) calcd for C₂₉H₃₂N₇O₃ (MH⁺) m/z 526.2593, found 526.2582. Anal. calcd for C₂₉H₃₁N₇O₃.0.5H₂O: C, 65.2; H, 6.0; N, 18.3; found: C, 65.0; H, 5.8; N, 18.1%.

### Example R

N-{3-[{3-[(1,4-Dioxido-1,2,4-benzotriazin-3-yl)amino]propyl}(methyl)amino]-propyl}-9-methyl-4-phenazinecarboxamide (45). A solution of 9-methylphenazine-4-carboxylic acid (130 mg, 0.53 mmol) and CDI (100 mg, 0.61 mmol) in DMF (5 ml) was stirred at 55° C. for 6 h. The solution was cooled to 20° C., diluted with dry benzene (10 mL), Sephadex LH-20 (300 mg) was added and the mixture stirred at 20° C. for 1 h. The mixture was filtered and the solvent evaporated. The residue was dissolved in dry THF (5 mL) and a solution of 40 (80 mg, 0.26 mmol) in THF (5 mL) added, and the solution stirred at 20° C. for 24 h. The solvent was evaporated and the residue purified by chromatography, eluting with a gradient (0-2%) of aqueous NH₃/(0-8%) MeOH/DCM, to give compound 45 (130 mg, 90%) as a red powder, mp (DCM/pet. ether) 138-142° C.; ¹H NMR δ 11.23 (br s, 1H, CONH), 8.84 (d, J=6.6 Hz, 1H, ArH), 8.29 (d, J=7.6 Hz, 1H, ArH), 8.07 (d, J=8.5 Hz, 1H, ArH), 8.04 (d, J=8.5 Hz, 1H, ArH), 7.98 (d, J=8.6 Hz, 1H, ArH), 7.85 (t, J=7.8 Hz, 1H, ArH), 7.78-7.71 (m, 3H, ArH, NH), 6.48 (t, J=7.6 Hz, 1H, ArH), 7.31 (t, J=7.7 Hz, 1H, ArH), 3.78-3.71 (m, 4H, 2×CH₂), 3.15 (br m, 4H, 2×CH₂), 2.88 (s, 3H, CH₃), 2.73 (br s, 3H, CH₃), 2.32, (br m, 2H, CH₂), 2.21 (br m, 2H, CH₂); ¹³C NMR δ 165.6, 149.6, 143.2, 142.9, 140.7, 139.4, 137.9, 136.4, 135.4, 135.1, 133.7, 131.3, 131.2, 130.1, 129.7, 128.5, 127.7, 127.0, 121.3, 116.9, 54.9, 54.2, 40.2, 38.9, 37.3, 25.7, 24.3, 18.1. Anal. calcd for C₂₈H₃₀N₈O₃: C, 63.9; H, 5.9; N, 21.3; HRMS (FAB⁺) calcd for (C₂₈H₃₁N₈O₃) (MH⁺) m/z 527.2519 found 527.2533. Anal. calcd for C₂₈H₃₀N₈O₃.1.75H₂O: C, 60.3; H, 6.0; N, 20.1; found: C, 60.3; H, 5.6; N, 19.6%.

### Example S

### N-{3-[(3-{[7-(2-Methoxyethoxy)-1,4-dioxido-1,2,4-benzotriazin-3-yl]amino}propyl)(methyl)amino]propyl}-4-acridinecarboxamide (55)

3-Amino-1,2,4-benzotriazin-7-ol 1-oxide (46). A mixture of 4-amino-3-nitrophenol (5.0 g, 32.4 mmol) and cyanamide (8.2 g, 194.6 mmol) was heated at 100° C. for 10 min. The resulted solution was cooled to 20° C. and c.HCl (15 mL) was added dropwise, and the mixture was heated at 100° C. for 1.5 h, cooled to 20° C. A solution of 30% NaOH (40 mL) was then added and heated at 100° C. for 1 h. The reaction mixture was cooled to 20° C., diluted with water (20 mL), and the precipitate was filtered, washed with water (100 mL), diethyl ether (100 mL), and dried to give amine 46 (5.45 g, 97%) as a yellow powder, mp>300° C. [lit. (Friebe et. al. U.S. Pat. No. 5,856,325, Jan. 5, 1999) mp (HOAc)>270° C.]; ¹H NMR [(CD₃)₂SO] δ 10.37 (br s, 1H, OH), 7.48 (dd, J=7.7, 2.6 Hz, 1H, H-6), 7.40-7.37 (m, 2H, H-5, H-8), 6.96 (br s, 2H, NH₂).

7-(2-Methoxyethoxy)-1,2,4-benzotriazin-3-amine 1-oxide (47). A mixture of 46 (1.00 g, 5.8 mmol), dry K₂CO₃ (2.40 g, 17.4 mmol) and 2-bromoethyl methyl ether (2.42 g, 17.4 mmol) in DMF (20 mL) was heated at 80° C. for 2 h. The solvent was evaporated and the residue purified by chromatography, eluting with a gradient (0-3%) of MeOH/DCM, to give compound 47 (1.06 g, 77%) as a yellow powder, mp (DCM/pet. ether) 201-203° C.; ¹H NMR [(CD₃)₂SO] δ 8.07(d, J=9.5 Hz, 1H, H-5), 7.82(br s, 2H, NH₂), 7.76 (dd, J=9.5, 2.6 Hz, 1H, H-6), 7.50 (d, J=2.6 Hz, 1H, H-8), 4.26, (t, J=4.3 Hz, 2H, CH₂), 3.72 (t, J=4.3 Hz, 2H, CH₂), 3.33 (s, 3H, OCH₃). Anal. calcd for C₁₀H₁₂N₄O₅: C, 50.8; H, 5.1; N, 23.7; found: C, 51.1; H, 5.0; N, 23.7%.

3-Hydroxy-7-(2-methoxyethoxy)-1,2,4-benzotriazine 1-oxide (48). A suspension of 47 (1.00 g, 4.2 mmol) in 2 N HCl (32 mL) was cooled to 5° C. and a solution of NaNO₂ (0.58 g, 8.5 mmol) in water (1.5 mL) was added over 1 h. More NaNO₂ (0.58 g, 8.5 mmol) in water (1.5 mL) was added and the suspension stirred 72 h at 20° C. The precipitate was filtered and washed with water. The solid was dissolved in 5% aqueous NH₃ and filtered. The filtrate was acidified with conc. HCl to give a precipitate which was filtered dried and purified by chromatography, eluting with a gradient (0-5%) of MeOH/DCM to give compound 48 (0.68 g, 68%) as a yellow solid, mp (DCM/pet. ether) 190-192° C.; ¹H NMR [(CD₃)₂SO] δ 12.52 (br, 1H, OH), 7.69 (br s, H, H-8), 7.53 (dd, J=8.8, 2.8 Hz, 1H, H-6), 7.33 (d, J=8.8 Hz, 1H, H-5), 4.19 (t, J=4.4 Hz, 2H, CH₂), 3.68 (t, J=4.4 Hz, 2H, CH₂), 3.33 (s, 3H, OCH₃); ¹³C NMR [(CD₃)₂SO] δ 154.6, 152.9, 131.8, 129.5, 127.4, 117.8, 101.8, 70.0, 67.9, 58.1. Anal. calcd for C₁₀H₁₁N₃O₄: C, 50.6; H, 4.2; N, 17.7; found: C, 50.5; H, 4.7; N, 17.7.

3-Chloro-7-(2-methoxyethoxy)-1,2,4-benzotriazine 1-oxide (49). A mixture of 48 (1.00 g, 4.3 mmol) in POCl₃ (8 mL) was refluxed for 2 h. Excess reagent was evaporated under vacuum, and ice cold water (50 mL) was added to the residue, then solid Na₂CO₃ (1.0 g) was added portionwise. The resulting precipitate was filtered and purified by chromatography, eluting with a gradient (50-100%) of DCM/pet. ether, to give compound 49 (0.90 g, 83%) as a pale yellow solid. mp (DCM/pet. ether) 121-125° C.; ¹H NMR [(CD₃)₂SO] δ 8.00 (d, J=9.2 Hz, 1H, H-5), 7.81 (dd, J=9.2, 2.9 Hz, 1H, H-6), 7.68 (d, J=2.8 Hz, 1H, H-8), 4.35 (t, J=4.4 Hz, 2H, CH₂), 3.74 (t, J=4.4 Hz, 2H, CH₂), 3.33 (s, 3H, OCH₃). Anal. calcd for C₁₀H₁₀ClN₃O₃: C, 47.0; H, 3.9; N, 16.4, Cl, 13.9; found: C, 46.9; H, 4.3; N, 16.4; Cl, 13.7.

N¹-(3-Aminopropyl)-N³-[7-(2-methoxyethoxy)-1-oxido-1,2,4-benzotriazin-3-yl]-N¹-methyl-1,3-propanediamine (51). A solution of chloride 49 (0.90 g, 3.5 mmol) tert-butyl 3-[(3-aminopropyl)(methyl)amino]propylcarbamate (50) (1.60 g, 5.25 mmol) and Et₃N (4 ml) in DME (20 mL) was heated to 90° C. for 4 h. The solvent was evaporated, the residue was dissolved in MeOH (10 mL), and treated with methanolic HCl (100 mL). The reaction mixture was stirred at 20° C. for 20 h, the solvent evaporated and the residue partitioned between DCM and dil. aqueous NH₃. The aqueous layer was extracted with DCM (4×25 mL), the combined extracts dried, and the solvent evaporated. The residue was purified by chromatography, eluting with a gradient (0-2%) of aqueous NH₃/(0-10%) MeOH/DCM, to give compound 51 (1.25 g, 98%) as a yellow solid, ¹H NMR [(CD₃)₂SO] δ 7.68 (br s, 1H, NH), 7.55-7.52 (m, 1H, ArH), 7.50-7.47 (m, 2H, ArH), 4.20 (t, J=4.4 Hz, 2H, CH₂), 3.70 (t, J=4.4 Hz, 2H, CH₂), 3.34 (br m, 2H, CH₂), 3.32 (s, 3H, OCH₃), 2.54 (br t, J=6.1H, 2H, CH₂), 2.35 (t, J=6.9 Hz, 2H, CH₂), 2.31 (t, J=7.2 Hz, 2H, CH₂), 2.13 (s, 3H, NCH₃), 1.70 (br quin, J=6.9 Hz, 2H, CH₂), 1.47 (br quin, J=7.0 Hz, 2H, CH₂); ¹³C NMR [(CD₃)₂SO] δ 158.3, 155.3, 144.5, 129.8, 138.3, 127.5, 98.9, 70.0, 67.7, 58.1, 55.0, 54.9, 41.8, 39.9, 39.1, 30.7, 26.2; HRMS (FAB⁺) calcd for C₁₇H₂₉N₆O₃ (MH⁺) m/z 365.2301, found 365.2311.

2,2,2-Trifluoro-N-{3-[(3-{[7-(2-methoxyethoxy)-1,4-dioxido-1,2,4-benzotriazin-3-yl]amino}propyl)(methyl)amino]propyl}acetamide (52). Ethyl trifluoroacetate (1.2 mL, 9.8 mmol) and H₂O (0.17 mL, 9.8 mmol) were added to a solution of 51 (1.19 g, 3.3 mmol) in CH₃CN and the reaction mixture was heated at reflux for 18 h. The solvent was evaporated and the residue partitioned between aqueous Na₂CO₃ Solution and DCM. The aqueous layer was extracted with DCM, the combined organic extracts dried and the solvent evaporated. The residue was purified by chromatography, eluting with a gradient (0-5%) of MeOH/DCM to give compound 52 (1.3 g, 87%) as a yellow solid, mp (DCM/pet. ether) 117-119° C.; ¹H NMR [(CD₃)₂SO] δ 9.43 (br s, 1H, CONH), 7.66 (br t, J=5.3 Hz, 1H, NH), 7.54-7.45 (m, 3H, ArH), 4.20 (t, J=4.4 Hz, 2H, CH₂), 3.70 (t, J=4.4 Hz, 2H, CH₂), 3.36-3.27 (m, 2H, CH₂), 3.30 (s, 3H, OCH₃), 3.21 (t, J=7.0 Hz, 2H, CH₂), 2.37 (t, J=6.9 Hz, 2H, CH₂), 2.31 (t, J=6.9 Hz, 2H, CH₂), 2.14 (s, 3H, NCH₃), 1.70 (br quin, J=7.0 Hz, 2H, CH₂), 1.63 (br quin, J=7.0 Hz, 2H, CH₂); ¹³C NMR [(CD₃)₂SO] δ 158.3, 156.5 (q, J=18 Hz), 155.3, 144.5, 129.8, 128.3, 127.5, 115.9 (q, J=288 Hz), 98.9, 70.0, 67.7, 58.1, 54.8, 54.5, 41.5, 39.0, 37.7, 26.2, 95.8; HRMS (EI⁺) calcd for C₁₉H₂₇F₃N₆O₄ (M⁺) m/z 460.2046, found 460.2040. Anal. calcd for C₁₉H₂₇F₃N₆O₄C, 49.6; H, 5.9; N, 18.3; F, 12.4; found: C, 49.9; H, 5.9; N, 18.2; F, 12.4%.

2,2,2-Trifluoro-N-{3-[(3-{[7-(2-methoxyethoxy)-1,4-dioxido-1,2,4-benzotriazin-3-yl]amino}propyl)(methyl)amino]propyl}acetamide (53). 70% H₂O₂ (1.05 mL, 21.7 mmol) was added dropwise to a solution of trifluoroacetic anhydride (3.0 mL, 21.7 mmol) in DCM (10 mL) at 5° C. The solution was stirred at 5° C. for 10 min, 20° C. for 10 min, and then cooled to 5° C. The solution was added dropwise to a solution of 1-oxide 52 (1.0 g, 2.2 mmol) and TFA (0.33 mL, 4.3 mmol) in DCM (50 mL). The reaction mixture was stirred at 20° C. for 18 h. The solution was partitioned between aqueous NaHCO₃ solution and DCM, the aqueous layer extracted further with DCM (5×30 mL), the combined extracts dried, and the solvent evaporated. The residue was chromatographed, eluting with a gradient (0-5%) of MeOH/DCM to give compound 53 (0.32 g, 30%) as a red solid, mp (DCM/pet. ether) 91-94° C.; ¹H NMR [(CD₃)₂SO] δ 9.43 (br s, 1H, CONH), 8.24 (t, J=5.6 Hz, 1H, NH,), 8.05 (d, J=9.5 Hz, 1H, H-5), 7.60 (dd, J=9.5, 2.7 Hz, 1H, H-6), 7.50 (d, J=2.6 Hz, 1H, H-8), 4.26 (t, J=4.3 Hz, 2H, CH₂), 3.72 (t, J=4.3 Hz, 2H, CH₂), 3.41 (br q, J=6.6 Hz, 2H, CH₂), 3.33 (s, 3H, OCH₃), 3.23 (br q, J=6.3 Hz, 2H, CH₂), 2.38 (t, J=6.7 Hz, 2H, CH₂), 2.32 (t, J=6.9 Hz, 2H, CH₂), 2.15 (s, 3H, NCH₃), 1.75 (br quin, J=6.7 Hz, 2H, CH₂), 1.65 (br quin, J=6.9 Hz, 2H, CH₂); ¹³C NMR [(CD₃)₂SO] δ 157.0, 156.0 (q, J=6 Hz), 149.0, 134.2, 130.2, 128.2, 120.2, 115.9 (q, J=279 Hz), 99.5, 69.9, 68.0, 58.1, 54.9, 54.6, 41.5, 39.5, 37.7, 25.9, 25.8; HRMS (FAB⁺) calcd for C₁₉H₂₈F₃N₆O₅ (MH⁺) m/z 477.2073, found 477.2074.

N-{3-[(3-{[7-(2-Methoxyethoxy)-1,4-dioxido-1,2,4-benzotriazin-3-yl]amino}propyl)(methyl)amino]propyl}-4-acridinecarboxamide (55). A solution of trifluoroacetamide 53 (1.55 g, 0.33 mmol) and aqueous NH₃ (8 mL) in MeOH (10 mL) was stirred at 20° C. for 18 h. The solvent was evaporated and the residue dried to give the intermediate amine 54 as a red solid. The solid was dissolved in dry THF (10 mL) and 4-(1H-imidazol-1-ylcarbonyl)acridine (0.18 g, 0.65 mmol) added and solution stirred at 20° C. for 72 h. The solvent was evaporated and the residue purified by chromatography, eluting with a gradient (0-2%) of aqueous NH₃/(0-5%) MeOH/DCM, to give compound 55 (150 mg, 79%) as a red solid, mp (DCM/pet. ether) 98-103° C.; ¹H NMR [(CD₃)₂SO] δ 11.32 (t, J=5.3 Hz, 1H, CONH), 9.27 (s, 1H, NH), 8.68 (dd, J=7.0, 1.5 Hz, 1H, ArH), 8.33 (dd, J=8.4, 1.3 Hz, 1H, ArH), 8.27-8.18 (m, 3H, ArH), 7.96-7.91 (m, 2H, ArH), 7.72 (dd, J=8.2, 7.2 Hz, 1H, ArH), 7.66 (t, J=7.3 Hz, 1H, ArH), 7.50 (dd, J=9.5, 2.6 Hz, 1H, ArH), 7.40 (d, J=2.6 Hz, 1H, ArH), 4.23 (t, J=4.3 Hz, 2H, CH₂), 3.71 (t, J=4.4 Hz, 2H, CH₂), 3.59 (br q, J=6.4 Hz, 2H, CH₂), 3.43 (br q, J=6.4 Hz, 2H, CH₂), 2.56 (t, J=7.0 Hz, 2H, CH₂), 2.46 (t, J=6.7 Hz, 2H, CH₂), 2.23 (s, 3H, NCH₃), 1.91 (br quin, J=6.8 Hz, 2H, CH₂), 1.78 (br quin, J=6.6 Hz, 2H, CH₂); ¹³C NMR [(CD₃)₂SO] δ 164.6, 156.9, 148.9, 146.9, 145.4, 138.5, 134.3, 134.1, 132.6, 131.8, 130.0, 128.5, 128.3, 128.3, 128.0, 126.4, 126.3, 125.5, 125.1, 118.4, 99.4, 69.9, 68.0, 58.1, 55.2, 55.0, 41.8, 39.7, 37.4, 26.9, 25.8; HRMS (FAB⁺) calcd for C₃₁H₃₆N₇O₅ (MH⁺) m/z 586.2778, found 586.2768. Anal. calcd for C₃₁H₃₅N₇O₅: C, 63.6; H, 6.0; N, 16.7; found: C, 62.3; H, 6.1; N, 16.5%.

### Example T

### N-{2-[[3-(1,4-Dioxido-1,2,4-benzotriazin-3-yl)propyl](methyl)amino]propyl}-4-acridinecarboxamide (62)

3-Allyl-1,2,4-benzotriazine 1-oxide (56). Pd(PPh₃)₄ (640 mg, 0.55 mmol) was added to a stirred solution of chloride 3 (2.0 g, 11.0 mmol) and allyltributyltin (3.8 mL, 12.1 mmol), the solution degassed, and stirred under N₂ at reflux temperature for 6 h. The solvent was evaporated and the residue purified by chromatography, eluting with 20% EtOAc/pet. ether to give an oil which was purified by chromatography, eluting with 5% EtOAc/DCM, to give alkene 56 (1.92 g, 93%) as a white solid, mp (EtOAc/pet. ether) 57-58° C., ¹H NMR δ 8.45 (dd, J=8.6, 1.4 Hz, 1H, H-8), 8.10 (dd, J=8.4, 1.4 Hz, 1H, H-5), 7.94 (ddd, J=8.4, 7.1, 1.4 Hz, 1H, H-6), 7.70 (ddd, J=8.6, 7.1, 1.4 Hz, 1H, H-7), 6.15-6.24 (m, 1H, H-2′), 5.31 (dq, J=17.0, 1.5 Hz, 1H, H-3′), 5.24 (dq, J=10.1, 1.5 Hz, 1H, H-3′), 3.80 (dq, J=6.8, 1.5 Hz, 2H, H-1′); ¹³C NMR 165.2, 147.5, 135.6, 133.3, 132.7, 130.1, 128.8, 120.8, 118.5, 41.8. Anal. calcd for C₁₀H₉N₃O: C, 64.2; H, 4.85; N, 22.45; found: C, 63.85; H, 4.9; N, 22.7%.

3-(3-Hydroxypropyl)-1,2,4-benzotriazine 1-oxide (57). A solution of 9-BBN in THF (13.7 mL, 6.8 mmol) was added to a stirred solution of alkene 56 (1.07 g, 5.7 mmol) in THF (50 mL) and the solution stirred at 20° C. for 1 h. A solution of NaOH (3 M; 2.9 ml, 8.5 mmol), followed by 35% H₂O₂ (2.6 mL, 25.6 mmol) were carefully added and the mixture stirred at 20° C. for 1 h. The mixture was diluted with brine (100 mL), extracted with EtOAc (3×100 mL), the combined organic fraction dried, and the solvent evaporated. The residue was purified by chromatography, eluting with a gradient (10-50%) of EtOAc/DCM, to give alcohol 57 (1.02 g, 87%) as a white solid, mp (EtOAc/pet. ether) 99-100° C.; ¹H NMR δ 8.46 (dd, J=8.7, 1.0 Hz, 1H, H-8), 7.99 (dd, J=8.5, 1.2 Hz, 1H, H-5), 7.93 (ddd, J=8.5, 7.0, 1.0 Hz, 1H, H-6), 7.70 (ddd, J=8.7, 7.0, 1.2 Hz, 1H, H-7), 3.80 (t, J=6.1 Hz, 2H, CH₂O), 3.18 (t, J=7.3 Hz, 2H, CH₂), 2.15-2.22 (m, 2H, CH₂), (OH not observed); ¹³C NMR δ 166.9, 147.3, 135.7, 133.3, 130.1, 128.6, 120.1, 62.1, 34.1, 30.5. Anal. calcd for C₁₀H₁₁N₃O₂: C, 58.5; H, 5.4; N, 20.5; found: C, 58.6; H, 5.5; N, 20.5%.

tert-Butyl 3-{methyl[3-(1-oxido-1,4-benzotriazin-3-yl)propyl]amino}propylcarbamate (58). MsCl (0.52 mL, 6.7 mmol) was added dropwise to a stirred solution of alcohol 57 (1.06 g, 5.2 mmol) and Et-N (1.1 mL, 7.8 mmol) in DCM (50 mL) and the solution stirred at 20° C. for 1 h. The solution was diluted with DCM (50 mL), washed with water (2×30 mL), dried, and the solvent evaporated. The residue was dissolved in dry DME (20 mL) and tert-butyl 3-(methylamino)propylcarbamate (Rennard et al. Org. Lett., 2000, 2, 2117-2120) (9.7 g, 51.6 mmol) added and the solution stirred at 50° C. for 3 h. The solvent was evaporated and the residue partitioned between EtOAc (100 mL) and aqueous KHCO₃ solution (100 mL). The organic fraction was washed with water (2×50 mL), dried, and the solvent evaporated. The residue was purified by chromatography, eluting with a gradient (0-10%) of MeOH/DCM, to give compound 58 (0.93 g, 48%) as a pale yellow oil, ¹H NMR δ 8.45 (dd, J=8.9, 1.4 Hz, 1H, H-8), 8.10 (br d, J=8.3 Hz, 1H, H-5), 7.93 (ddd, J=8.3, 7.0, 1.4 Hz, 1H, H-6), 7.70 (ddd, J=8.9, 7.0, 1.5 Hz, 1H, H-7), 5.38 (br s, 1H, NH), 3.17-3.22 (m, 2H, CH₂N), 3.07 (dd, J=7.7, 7.4 Hz, 2H, CH₂), 2.55-2.60 (m, 2H, CH₂N, 2.49-2.53 (m, 2H, CH₂N), 2.28 (s, 3H, NCH₃), 2.10-2.18 (m, 2H, CH₂), 1.68-1.73 (m, 2H, CH₂), 1.42 [s, 9H, C(CH₃)₃]; ¹³C NMR δ 166.7, 156.1, 147.5, 135.6, 133.3, 130.0, 128.7, 120.1, 78.9, 56.7, 55.6, 41.5, 39.3, 34.9, 28.3 (3), 26.6, 25.0; MS (FAB⁺) m/z 376 (MH⁺, 55%), 360 (5); HRMS (FAB⁺) calcd for C₁₉H₃₀N₅O₃ (MH⁺) m/z 376.2349, found 376.2345.

2,2,2-Trifluoro-N-(3-{methyl[3-(1-oxido-1,2,4-benzotriazin-3-yl)propyl]amino}propyl)acetamide (59). A solution of carbamate 58 (0.51 g, 1.35 mmol) in HCl saturated MeOH (30 mL) was stirred at 50° C. for 3 h. The solvent was evaporated and the residue partitioned between dil. aqueous NH₃ (50 ml) and CHCl₃ (50 mL). The aqueous fraction was extracted with CHCl₃ (3×30 mL), the combined organic fraction dried, and the solvent evaporated. The residue was dissolved in MeCN (30 mL) and ethyl trifluoroacetate (0.24 mL, 2.03 mmol) and water (30 μL, 1.5 mmol) added. The solution was stirred at reflux temperature for 16 h, cooled to 20° C. and the solvent evaporated. The residue was purified by chromatography, eluting with a gradient (0-10%) of MeOH/DCM, to give amide 59 (460 mg, 92%) as a pale yellow oil, ¹H NMR [(CD₃)₂SO] δ 9.41 (br s, 1H, CONH), 8.37 (d, J=8.6 Hz, 1H, H-8), 8.07 (ddd, J=8.3, 6.9, 1.4 Hz, 1H, H-6), 8.02 (dd, J=8.3, 1.3 Hz, 1H, H-5), 7.83 (ddd, J=8.6, 6.9, 1.3 Hz, 1H, H-7), 3.17-3.22 (m, 2H, CH₂N), 2.95 (dd, J=7.6, 7.4 Hz, 2H, CH₂), 2.42 (br t, J=6.8 Hz, 2H, CH₂N), 2.33 (br t, J=6.7 Hz, 2H, CH₂N), 2.16 (s, 3H, NCH₃), 1.92-2.00 (m, 2H, CH₂), 1.57-1.64 (m, 2H, CH₂); ¹³C NMR [(CD₃)₂SO] δ 166.2, 156.0 (q, J=37 Hz), 146.9, 136.0, 132.8, 130.4, 128.3, 119.5, 115.9 (q, J=288 Hz), 56.1, 54.4, 41.4, 37.6, 34.2, 25.7, 24.8; MS (EI⁺) m/z 371 (M⁺, 7%), 354 (100); HRMS (EI⁺) calcd for C₁₆H₂₀F₃N₅O₂ (M⁺) m/z 371.1569, found 371.1560.

N-{3-[[3-(1,4-Dioxido-1,2,4-benzotriazin-3-yl)propyl](methyl)amino]propyl}-2,2,2-trifluoroacetamide (60). H₂O₂ (0.6 mL, 12.2 mmol) was added dropwise to a stirred solution of trifluoroacetic anhydride (1.7 mL, 12.2 mmol) in DCM (10 mL) at 5° C. The mixture was stirred at 5° C. for 5 min., warmed to 20° C. for 20 min., cooled to 5° C., and added to a stirred solution of amide 59 (453 mg, 1.2 mmol) and trifluoroacetic acid (0.19 mL, 2.4 mmol) in CHCl₃ (10 mL) at 5° C. The mixture was stirred at 20° C. for 4 h, diluted with aqueous KHCO₃ (15 mL), and extracted with CHCl₃ (5×30 mL). The combined organic fraction was dried, adsorbed on to silica, and the solvent evaporated (CAUTION: use blast shield). The residue was purified by chromatography, eluting with a gradient (0-10%) of MeOH/DCM, to give 1,4-dioxide 60 (268 mg, 57%) as a yellow oil, ¹H NMR [(CD₃)₂SO] δ 9.40 (br s, 1H, NHCO), 8.34-8.38 (m, 2H, H-5, H-8), 8.10 (ddd, J=8.7, 7.1, 1.2 Hz, 1H, H-6), 7.94 (ddd, J=8.5, 7.1, 1.3 Hz, 1H, H-7), 3.16-3.21 (m, 2H, CH₂N), 3.04 (dd, J=7.6, 7.4 Hz, 2H, CH₂), 2.43 (br t, 6.8 Hz, 2H, CH₂N), 2.32 (br t, J=6.8 Hz, 2H, CH₂), 2.14 (s, 3H, NCH₃), 1.87-1.94 (m, 2H, CH₂), 155-1.62 (m, 2H, CH₂); ¹³C NMR [(CD₃)₂SO] δ 155.9 (q, J=37 Hz), 154.7, 139.3, 135.4, 134.4, 131.7, 120.9, 118.8, 115.8 (q, J=288 Hz), 56.2, 54.3, 41.3, 37.6, 27.6, 25.8, 21.8; MS (FAB⁺) m/z 388 (MH⁺, 25%), 372 (5); HRMS (FAB⁺) calcd for C₁₆H₂₁F₃N₅O₃ (MH⁺) m/z 388.1597, found 388.1601.

N¹-[3-(1,4-Dioxido-1,2,4-benzotriazin-3-yl)propyl]-N¹-methyl-1,3-propanediamine (61). Aq. ammonia (5 mL) was added to a stirred solution of amide 60 (169 mg, 0.44 mmol) in MeOH (10 mL) and the solution stirred at 40° C. for 6 h. The solvent was evaporated to give crude amine 61 as a brown oil, ¹H NMR [(CD₃)₂SO] δ 8.34-8.39 (m, 2H, H-5, H-8), 8.14 (ddd, J=8.6, 7.0, 1.1 Hz, 1H, H-6), 7.96 (ddd, J=8.5, 7.0, 1.2 Hz, 1H, H-7), 7.61 (br s, 2H, NH₂), 3.04 (dd, J=7.6, 7.4 Hz, 2H, CH₂N), 2.85 (br dd, J=7.4, 7.2 Hz, 2H, CH₂), 2.45 (br t, J=6.9 Hz, 2H, CH₂N), 2.39 (br t, J=6.7 Hz, 2H, CH₂N), 2.17 (s, 3H, NCH₃), 1.88-1.95 (m, 2H, CH₂), 1.63-170 (m, 2H, CH₂).

N-{3-[[3-(1,4-dioxido-1,2,4-benzotriazin-3-yl)propyl](methyl)amino]propyl}-4-acridinecarboxamide (62). The crude amine 61 was dissolved in dry THF (10 mL) and 4-(1H-imidazol-1-ylcarbonyl)acridine (0.18 g, 0.65 mmol) added and solution stirred at 20° C. for 16 h. The solvent was evaporated and the residue purified by chromatography, eluting with a gradient (0-10%) of MeOH/DCM, to give compound 62 (86 mg, 40%) as a yellow gum, which was converted to the hydrochloride salt, a pale green gum, ¹H NMR [(CD₃)₂SO] δ 11.29 (br s, 1H, CONH), 10.90 (br s, 1H, NH⁺Cl⁻), 9.38 (s, 1H, H-9), 8.74 (dd, J=7.0, 1.0 Hz, 1H, H-3), 8.47 (d, J=8.7 Hz, 1H, H-1), 8.42 (dd, J=8.4, 1.2 Hz, 1H, H-5), 8.35 (dd, J=8.6, 0.7 Hz, 1H, H-8′), 8.31 (d, J=8.7 Hz, 1H, H-8), 8.21 (d, J=8.4 Hz, 1H, H-5′), 8.11 (ddd, J=8.7, 7.0, 1.3 Hz, 1H, H-6), 7.94-8.10 (m, 2H, H-2, H-6′), 7.78 (dd, J=8.7, 7.0 Hz, 1H, H-7), 7.67-7.71 (m, 1H, H-7′), 3.65-3.70 (m, 2H, CH₂N), 3.30-3.37 (m, 2H, CH₂N), 3.19-3.28 (m, 2H, CH₂N), 3.09 (t, J=7.3 Hz, 2H, CH₂), 2.79 (d, J=4.8 Hz, 3H, NCH₃), 2.16-2.27 (m, 4H, 2×CH₂); ¹³C NMR [(CD₃)₂SO] δ 165.3, 153.0, 146.3, 144.7, 139.6, 139.2, 135.5, 134.7, 134.5, 134.0, 133.0, 132.3, 132.0, 128.4, 128.1, 126.6, 126.3, 125.5, 125.3, 120.9, 118.8, 53.7, 52.7, 39.4, 36.4, 26.8, 23.7, 18.8; MS (FAB⁺) m/z 497 (MH⁺, 12%), 481 (3); HRMS (calcd for C₂₈H₂₉N₆O₃ (MH⁺) m/z 497.2301, found 497.2301.

### Example U

N-{3-[[3-(1,4-Dioxido-1,2,4-benzotriazin-3-yl)propyl](methyl)amino]propyl}-1-phenazinecarboxamide (63). Aq. ammonia (5 mL) was added to a stirred solution of amide 60 (61 mg, 0.16 mmol) in MeOH (10 mL) and the solution stirred at 40° C. for 6 h. The solvent was evaporated to give crude amine 61 as a brown oil. The crude amine 61 was dissolved in dry THF (10 mL) and 1-(1H-imidazol-1-ylcarbonyl)phenazine (100 mg, 0.36 mmol) added and solution stirred at 20° C. for 16 h. The solvent was evaporated and the residue purified by chromatography, eluting with a gradient (0-10%) of MeOH/DCM, to give compound 63 (44 mg, 56%) as a yellow gum, which was converted to the hydrochloride salt and recrystallised, mp MeOH/EtOAc) 173° C. (dec.); ¹H NMR [(CD₃)₂SO] δ 10.31 (t, J=5.8 Hz, 1H, NH), 9.90 (br s, 1H, NH⁺Cl⁻), 8.61 (dd, J=7.1, 1.4 Hz, 1H, H-2), 8.48 (dd, J=9.1, 1.4 Hz, 1H, H-9), 8.42 (dd, J=8.6, 1.4 Hz, 1H, H-4), 8.34 (d, J=8.4 Hz, 1H, H-6), 8.30 (dd, J=8.6, 1.1 Hz, 1H, H-8′), 8.26 (dd, J=8.3, 1.4 Hz, 1H, H-5′), 7.93-8.13 (m, 5H, H-3, H-7, H-8, H-6′, H-7′), 3.62-3.67 (m, 2H, CH₂N), 3.30-3.34 (m, 2H, CH₂N), 3.22-3.38 (m, 2H, CH₂N), 3.07-3.11 (m, 2H, CH₂), 2.82 (br s, 3H, NCH₃), 2.10-2.22 (m, 4H, 2×CH₂); ¹³C NMR [(CD₃)₂SO] δ 164.8, 153.1, 142.7, 142.5, 141.2, 140.0, 139.2, 135.6, 134.5, 133.5, 132.7, 132.0, 131.9, 131.6, 130.9, 130.3, 129.4, 129.1, 121.0, 118.8, 5.0, 52.9, 39.4, 36.4, 26.7, 23.8, 19.0; MS (FAB⁺) m/z 498 (MH⁺, 20%), 482 (5); HRMS (FAB⁺) calcd for C₂₇H₂₈N₇O₃ (MH⁺) m/z 498.2254, found 498.2256.

### Example V

### 3-[(7-Chloro-4-quinolinyl)amino]-N-{3-[(1,4-dioxido-1,2,4-benzotriazin-3-yl)amino]propyl}propanamide (65)

A solution of N-(7-chloro-4-quinolinyl)-β-alanine (64) (Titus et al, J. Org. Chem., 1948, 13, 39-62) (303 mg, 1.2 mmol) and CDI (235 mg, 1.5 mmol) in DMF (5 mL) was stirred at 50° C. for 1 h. The solvent was evaporated and the residue crystallised from DCM/pet. ether to give the imidazolide (290 mg, 80%), which was used directly. A solution of N¹-(1,4-dioxido-1,2,4-benzotriazin-3-yl)-1,3-propanediamine (16) (92 mg, 390 μmol) and imidazolide (176 mg, 590 mmol) in DMF (10 mL) was stirred at 20° C. for 3 days, the solvent evaporated and the residue recrystallised from hot MeOH to give compound 65 (84 mg, 46%) as a red powder, mp (MeOH) 202° C. (dec.); ¹H NMR [(CD₃)₂SO] δ 8.40 (d, J=5.4 Hz, 1H, H-2′), 8.26 (br t, J=6.2 Hz, 1H, NH), 8.18-8.12 (m, 2H, H-5, H-8), 8.13 (d, J=8.6 Hz, 1H, H-5′), 7.99 (br t, J=5.7 Hz, 1H, NH), 7.93 (ddd, J=8.6, 7.1, 1.2 Hz, 1H, H-6), 7.75 (d, J=2.2 Hz, 1H, H-8′), 7.56 (ddd, J=8.6, 7.1, 1.3 Hz, 1H, H-7), 7.40 (dd, J=8.6, 2.2 Hz, 1H, H-6′), 7.37 (br t, J=5.4 Hz, 1H, NH), 6.52 (d, J=5.4 Hz, 1H, H-3′), 3.49-3.54 (m, 2H, CH₂N), 3.36-3.41 (m, 2H, CH₂N), 3.12-3.17 (m, 2H, CH₂N), 2.47-2.51 (m, 2H. CH₂), 1.70-1.77 (m, 2H, CH₂); ¹³C NMR [(CD₃)₂SO] δ 170.3, 151.8, 149.7, 149.6, 149.0, 138.1, 135.4, 133.2, 129.8, 127.4, 126.8, 124.0, 123.9, 121.0, 117.4, 116.8, 99.7, 39.0, 38.2, 35.8, 34.3, 28.5; MS (FAB⁺) m/z 470 (MH⁺, 5%), 468 (15), 454 (1), 452 (3): HRMS (FAB⁺) calcd for C₂₂H₂₃³⁵ClN₇O₃ (MH⁺) m/z 468.1551, found 468.1546; calcd for C₂₂H₂₃³⁷ClN₇O₃ (MH⁺) m/z 470.1540, found 470.1535.

### Example W

### N-[3-(Methyl{3-[(7-methyl-1,4-dioxido-1,2,4-benzotriazin-3-yl)amino]propyl}amino)propyl]-4-acridinecarboxamide (74)

7-Methyl-1,2,4-benzotriazin-3-ol 1-Oxide (67). NaNO₂ (9.0 g, 130.6 mmol) was added in small portions to a stirred solution of 7-methyl-1,2,4-benzotriazin-3-amine 1-oxide [Hay et al, J. Med. Chem. 2003, 46, 169-182] (66) (11.5 g, 65.3 mmol) in TFA (40 mL) at −5 to 0° C. After the addition was completed stirring was continued for a further 1 h, the mixture was poured into ice (300 g) and stirred 1 h. The resulting pale yellow precipitate was filtered and washed with water. The precipitate was dissolved in 8% aqueous NH₃, filtered and the filtrate was acidified with cHCl. The resulting precipitate was filtered, washed with water and dried to give alcohol 67 (11.5 g, 100%) which was used without further purification.

3-Chloro-7-methyl-1,2,4-benzotriazine 1-Oxide (68). Alcohol 67 (3.15 g, 65.3 mmol) was refluxed in POCl₃ (50 mL) for 5 h. The reaction mixture was cooled and carefully poured into ice/water and stirred for 30 min. The resulting precipitate was filtered, air dried and purified by chromatography, eluting with a gradient (50-100%) of DCM/hexane, to give chloride 68 (9.0 g, 66%), mp (DCM/hexane) 174-176° C.; ¹H NMR δ 8.20 (br s, 1H, H-8), 7.89 (d, J=8.6 Hz, 1H, H-5), 7.82 (dd, J=8.6, 1.9 Hz, 1H, H-6), 2.61 (s, 3H, CH₃). Anal. calcd for C₈H₆ClN₃O: C, 49.1; H, 3.1; N, 21.5; Cl, 18.1, found: C, 49.1, H, 3.1; N, 21.5; Cl, 18.5%.

tert-Butyl 3-(Methyl-{3-[(7-methyl-1-oxido-1,2,4-benzotriazin-3-yl)amino]propyl}amino)propylcarbamate (70). A mixture of chloride 68 (2.18 g, 11.1 mmol), tert-butyl 3-[(3-aminopropyl)(methyl)amino]propylcarbamate 69 (4.35 g, 17.8 mmol) and Et₃N (2.3 mL, 16.5 mmol) in DME (25 mL) was heated at 85° C. for 3 h. The solvent was evaporated, the residue was dissolved in DCM (100 mL) and washed with aqueous NH₃. The organic layer was separated and the aqueous layer further extracted with DCM (3×30 mL). The combined organic fraction was dried and the solvent evaporated. The residue was purified by chromatography, eluting with a gradient (0-1%) of aqueous NH₃/(0-5%) MeOH/DCM, to give 70 (3.7 g, 93%) as a yellow solid, mp (DCM/hexane) 117-120° C.; ¹H NMR [(CD₃)₂SO] δ 7.94 (s, 1H, H-8), 7.79 (br s, 1H, NH), 7.62 (dd, J=8.7, 2.0 Hz, 1H, H-6), 7.49 (d, J=8.6 Hz, 1H, H-5), 6.75 (t, J=5.3 Hz, 1H, NH), 3.34 (br q, J=6.4 Hz, 2H, CH₂), 2.93 (br q, J=6.5 Hz, 2H, CH₂), 2.41 (s, 3H, CH₃), 2.35 (t, J=6.9 Hz, 2H, CH₂), 2.27 (t, J=7.0 Hz, 2H, CH₂), 2.12 (s, 3H, CH₃), 1.70 (br quin, J=6.9 Hz, 2H, CH₂), 1.51 (br quin, J=7.1 Hz, 2H, CH₂), 1.35 (s, 9H, 3×CH₃); ¹³C NMR [(CD₃)₂SO] δ 158.6, 155.4, 146.8, 137.5, 134.4, 129.5, 125.7, 118.4, 77.2, 54.8, 54.7, 41.6, 39.0, 38.2, 28.1 (3) 27.1, 26.1, 20.6.

N¹-(3-Aminopropyl)-N¹-methyl-N³-(6-methyl-1-oxido-1,2,4-benzotriazin-3-yl)-1,3-propanediamine (71). Carbamate 70 (4.1 g 10.1 mmol) was dissolved in methanolic HCl (50 mL) and stirred for 48 h at 20° C. Excess reagent and solvent were evaporated and the residue was partitioned between DCM and aqueous NH₃, the organic layer was separated and the aqueous layer was further extracted with DCM (4×30 mL). The combined organic fraction was dried and the solvent evaporated. The residue was purified by chromatography, eluting with a gradient (01%) of aqueous NH₃/(3-10%) MeOH/DCM, to give amine 71 (1.14 g, 100%) as a yellow solid, ¹H NMR [(CD₃)₂SO] δ 7.94 (s, 1H, H-8), 7.81 (br s, 1H, NH), 7.63 (dd, J=8.7, 1.9 Hz, 1H, H-6), 7.49 (d, J=8.6 Hz, 1H, H-5), 3.34 (br q, J=6.3 Hz, 2H, CH₂), 2.54-2.58 (m, 2H, CH₂), 2.41 (s, 3H, CH₃), 2.36 (t, J=6.9 Hz, 2H, CH₂), 2.31 (t, J=7.2 Hz, 2H, CH₂), 2.13 (s, 3H, CH₃), 1.71 (br quin, J=7.0 Hz, 2H, CH₂), 1.48 (br quin, J=7.0 Hz, 2H, CH₂), NH₂ not observed; HRMS (FAB⁺) calcd for C₁₅H₂₅N₆O (MH⁺) m/z 305.2090, found 305.2090.

2,2,2-Trifluoro-N-[3-(methyl-{3-[(7-methyl-1-oxido-1,2,4-benzotriazin-3-yl)amino]propyl}amino)propyl]acetamide (72). CF₃CO₂Et (2.43 mL, 20.4 mmol) and H₂O (0.36 mL, 20.4 mmol) were added to a solution of amine 71 (3.1 g, 10.2 mmol) in CH₃CN (50 mL) and the reaction mixture heated at reflux for 20 h. The solvent was evaporated and residue partitioned between DCM and aqueous NaHCO₃. The organic layer was separated and the aqueous layer was further extracted with DCM (3×50 mL). The combined organic fraction was dried and the solvent evaporated to give acetamide 72 (3.75 g, 92%) as a yellow solid, mp (DCM/hexane) 121-124° C., ¹H NMR [(CD₃)₂SO] δ 9.44 (br s, 1H, NH), 7.94 (s, 1H, H-8), 7.80 (br s, 1H, NH), 7.62 (dd, J=8.7, 1.9 Hz, 1H, H-6), 7.48 (d, J=8.6 Hz, 1H, H-5), 3.22 (br q, J=6.5 Hz, 2H, CH₂), 2.41 (s, 3H, CH₃), 2.37 (t, J==7.0 Hz, 2H, CH₂), 2.32 (t, J=6.9 Hz, 2H, CH₂), 2.15 (s, 3H, CH₃), 1.71 (br quin, J=7.0 Hz, 2H, CH₂), 1.63 (br quin, J=6.5 Hz, 2H, CH₂), CH₂ not observed; ¹³C NMR [(CD₃)₂SO] δ 158.6, 156.3 (q, J=36 Hz), 146.8, 137.6, 134.5, 129.6, 125.7, 118.4, 116.1 (q, J=288 Hz), 54.7, 54.5, 41.5, 39.0, 37.8, 26.1, 25.8, 20.6; HRMS (FAB⁺) calcd for C₁₇H₂₄F₃N₆O₂ (MH⁺) m/z 401.1913, found 401.1896. Anal. calcd for C₁₇H₂₃F₃N₆O₂: C, 51.0; H, 5.8; F, 14.2; N, 21.0; found: C, 51.3; H, 5.9; F, 14.0; 21.0%.

2,2,2-Trifluoro-N-[3-(methyl{3-[(7-methyl-1,4-dioxido-1,2,4-benzotriazin-3-yl)amino]propyl}amino)propyl]acetamide (73). A solution of trifluoroperacetic acid [made from trifluoroacetic anhydride (12.4 mL, 89.4 mmol) and 70% H₂O₂ (4.3 mL, 89.4 mmol) in DCM (50 mL)] was added to a solution of acetamide 72 (3.6 g; 8.9 mmol) and trifluoroacetic acid (2.8 mL, 35.8 mol) in DCM (50 mL) at 0° C. and the reaction mixture was stirred at 20° C. for 18 h. The reaction mixture was slowly added to a solution of aqueous NaHCO₃ (100 mL) at 5° C. The organic layer was separated and the aqueous layer was further extracted with DCM (4×30′ mL). The combined organic fraction was dried and the solvent evaporated. The residue was purified by chromatography, eluting with a gradient (0-8%) of MeOH/DCM, to give (i) starting material (1.44 g, 40%) and (ii) acetamide 73 (1.30 g, 35%) as a red solid, mp (DCM/hexane) 117-119° C.; ¹H NMR [(CD₃)₂SO] δ 9.44 (br s, 1H, NH), 8.36 (t, J=5.9 Hz, 1H, NH), 8.03 (d, J=8.9 Hz, 1H, H-5), 8.01 (s, 1H, H-8), 7.78 (dd, J=8.9, 1.6 Hz, 1H, H-6), 3.42 (br q, J=6.6 Hz, 2H, CH₂), 3.23 (br q, J=6.5 Hz, 2H, CH₂), 2.47 (s, 3H, CH₃), 2.38 (t, J=6.7 Hz, 2H, CH₂), 2.32 (t, J=6.9 Hz, 2H, CH₂), 2.15 (s, 3H, CH₃), 1.75 (br quin, J=6.9 Hz, 2H, CH₂), 1.65 (br quin, J=7.1 Hz, 2H, CH₂); ¹³C NMR [(CD₃)₂SO] δ 156.0 (q, J=36 Hz), 149.4, 137.4, 137.2, 136.7, 129.6, 119.4, 116.6, 115.9 (q, J=288 Hz), 54.9, 54.6, 41.5, 39.5, 37.6, 26.0, 25.8, 20.7; HRMS (FAB⁺) calcd for C₁₇H₂₄F₃N₆O₃ (MH⁺) m/z 417.1862, found 417.1859. Anal. calcd for C₁₇H₂₃F₃N₆O₃: C, 49.0; H, 5.6; F, 13.7; N, 20.2; found: C, 49.3; H, 5.5; F, 13.6; N, 20.2%.

N-[3-(Methyl{3-[(7-methyl-1,4-dioxido-1,2,4-benzotriazin-3-yl)amino]propyl}amino)propyl]-4-acridinecarboxamide (74). Aqueous NH₃ (5 mL) was added to a solution of acetamide 73 (135 mg, 0.32 mmol) in MeOH (10 mL) and the reaction mixture stirred at 20° C. for 18 h. The solvent was evaporated, the residue was dissolved in DMF (5 mL), 4-(1H-imidazol-1-ylcarbonyl)acridine (177 mg, 0.64 mmol) was added and the mixture stirred at 20° C. for 48 h. The solvent was evaporated and the residue was purified by chromatography, eluting with a gradient (0-1%) of aqueous NH₃/(0-5%) MeOH/DCM, to give compound 74 (168 mg, 100%) as a red solid. mp (DCM/hexane) 166-168° C.; ¹H NMR [(CD₃)₂SO] δ 11.32 (br s, 1H, NH), 9.26 (s, 1H, ArH), 8.68 (d, J=6.7 Hz, 1H, ArH), 8.37 (t, J=5.6 Hz, 1H, NH), 8.33 (d, J=8.0 Hz, 1H, ArH), 8.23 (d, J=8.7 Hz, 1H, ArH), 8.17 (d, J=8.4 Hz, 1H, ArH), 7.89-7.96 (m, 3H, ArH), 7.64-7.74 (m, 3H, ArH), 3.59 (br q, J=6.0 Hz, 2H, CH₂), 3.41 (br q, J=6.2 Hz, 2H, CH₂), 2.56 (t, J=7.0 Hz, 2H, CH₂), 2.46 (t, J=6.9 Hz, 2H, CH₂), 2.44 (s, 3H, CH₃), 2.23 (s, 3H, CH₃), 1.91 (br quin, J=6.7 Hz, 2H, CH₂), 1.79 (br quin, J=6.6 Hz, 2H, CH₂); ¹³C NMR [(CD₃)₂SO] δ 164.7, 149.2, 147.0, 145.4, 138.5, 17.2, 137.1, 136.5, 134.3, 132.6, 131.8, 129.4, 128.5, 128.4, 128.3, 126.4, 126.4, 125.5, 125.2, 119.3, 116.5, 55.3, 55.1, 41.8, 39.5, 37.4, 26.9, 25.9, 20.7; HRMS (FAB⁺) calcd for C₂₉H₃₂N₇O₃ (MH⁺) m/z 526.2567, found 526.2537. Anal. calcd for C₂₉H₃₁N₇O₃.¼H₂O: C, 65.7; H, 6.0; N, 18.5; found: C, 65.8; H, 5.9; N, 18.7%.

### Example X

N-[3-(Methyl{3-[(7-methyl-1,4-dioxido-1,2,4-benzotriazin-3-yl)amino]propyl}amino)propyl]-2-(4-pyridinyl)-8-quinolinecarboxamide (75). Aqueous NH₃ (5 mL) was added to a solution of acetamide 73 (135 mg, 0.32 mmol) in MeOH (5 mL) and the mixture stirred at 20° C. for 18 h. The solvent was evaporated, the residue dissolved in DME (5 mL) and 8-(1H-imidazol-1-ylcarbonyl)-2-(4-pyridinyl)quinoline (160 mg, 0.64 mmol) was added and the mixture stirred at 20° C. for 48 h. The solvent was evaporated, the residue dissolved in DCM (20 mL) and washed with water (3×15 mL). The organic layer was separated, dried and the solvent evaporated. The residue was purified by chromatography, eluting with a gradient (0-1%) of aqueous NH₃/(0-3%) MeOH/DCM, to give compound 75 (158 mg, 89%) as a red solid, mp (DCM/hexane) 178-180° C.; ¹H NMR δ 10.89 (t, J=4.9 Hz, 1H, NH), 8.80-8.84 (m, 3H, ArH), 8.36 (d, J=8.6 Hz, 1H, ArH), 8.28 (t, J=4.8 Hz, 1H, NH), 8.05 (s, 1H, ArH), 8.02 (d, J=8.9 Hz, 1H, ArH), 7.90-7.95 (m, 4H, ArH), 7.67 (t, J=7.7 Hz, 1H, ArH), 7.56 (dd, J=8.9, 1.7 Hz, 1H, ArH), 4.73 (br q, J=6.5 Hz, 2H, CH₂), 3.52 (br q, J=6.0 Hz, 2H, CH₂), 2.54 (J=7.3 Hz, 2H, CH₂), 2.49 (s, 3H, CH₃), 2.47 (t, J=6.3 Hz, 2H, CH₂), 2.24 (s, 3H, CH₃), 2.01 (br quin, J=7.1 Hz, 2H, CH₂), 1.74 (br quin, J=6.2 Hz, 2H, CH₂); ¹³C NMR δ 165.8, 154.4, 150.8 (2), 149.4, 146.2, 145.4, 138.9, 137.7, 137.6, 136.8, 134.3, 131.2, 130.0, 129.9, 127.9, 127.2, 121.7 (2), 120.1, 118.6, 117.0, 56.6, 55.7, 41.9, 41.3, 38.2, 27.7, 25.6, 21.4; HRMS (FAB⁺) calcd for C₃₀H₃₃N₈O₃ (MH⁺) m/z 553.2676, found 553.2669. Anal. calcd for C₃₀H₃₂N₈O₃.½H₂O: C, 64.2; H, 5.9; N, 20.0; found: C, 64.0; H, 5.7; N, 20.0%.

### Example Y

N-[3-(Methyl{3-[(7-methyl-1,4-dioxido-1,2,4-benzotriazin-3-yl)amino]propyl}amino)propyl]-1-phenazinecarboxamide (76). Aqueous NH₃ (6 mL) was added to a solution of acetamide 73 (141 mg, 0.34 mmol) in MeOH (10 mL) and the mixture stirred at 20° C. for 18 h. The solvent was evaporated, the residue was dissolved in DMF (5 mL) and 1-(1H-imidazol-1-ylcarbonyl)phenazine (183 mg, 0.68 mmol) was added and mixture stirred at 20° C. for 48 h. The solvent was evaporated and the residue was purified by chromatography, eluting with a gradient (0-1%) of aqueous NH₃/(0-4%) MeOH/DCM, to give compound 76 (178 mg, 100%) as a red solid, mp (DCM/hexane) 118-122° C.; ¹H NMR δ 10.86 (br s, 1H, NH), 8.93 (dd, J=7.0, 1.5, 1H, ArH), 8.45 (br s, 1H, NH), 8.34 (dd, J=8.7, 1.5 Hz, 1H, ArH), 8.21-8.27 (m, 2H, ArH), 8.30 (s, 1H, ArH), 7.99 (d, J=8.8 Hz, 1H, ArH), 7.93 (dd, J=8.7, 7.2 Hz, 1H, ArH), 7.79-7.83 (m, 2H, ArH), 7.53 (dd, J=8.9, 1.8 Hz, 1H, ArH), 3.77 (br q, J=6.4 Hz, 2H, CH₂), 3.65 (br q, J=5.9 Hz, 2H, CH₂), 2.67 (t, J=7.4 Hz, 2H, CH₂), 2.62 (t, J=6.1 Hz, 2H, CH₂), 2.49 (s, 3H, CH₃), 2.35 (s, 3H, CH₃), 2.09 (br quin, J=7.1 Hz, 2H, CH₂), 1.88 (br quin, J=6.2 Hz, 2H, CH₂); ¹³C NMR δ 165.1, 149.4, 143.4, 142.9, 141.3, 140.8, 137.8, 137.7, 136.7, 135.1, 135.0, 133.4, 131.6, 131.0, 130.0, 129.8, 129.7, 129.0, 120.1, 116.8, 56.8, 55.8, 42.0, 41.5, 38.2, 27.5, 25.6, 21.4; HRMS (FAB⁺) calcd for C₂₈H₃₁N₈O₃ (MH⁺) m/z 527.2519, found 527.2512. Anal. calcd for C₂₈H₃₀N₈O₃.¼H₂O: C, 63.3; H, 5.8; N, 21.1; found: C, 63.2, H, 5.9, N, 21.4%.

### Example Z

9-Methyl-N-[3-({3-[(7-methyl-1,4-dioxido-1,2,4-benzotriazin-3-yl)amino]propyl}amino)propyl]-1-phenazinecarboxamide (77). Aqueous NH₃ (5 mL) was added to a solution of acetamide 73 (135 mg, 0.32 mmol) in MeOH (5 mL) and the solution stirred at 20° C. for 18 h. The solvent was evaporated, the residue dissolved in DMF (5 mL) and 1-(1H-imidazol-1-ylcarbonyl)-9-methylphenazine (172 mg, 0.6 mmol) was added and stirred at 20° C. for 48 h. The solvent was evaporated and the residue was purified by chromatography, eluting with a gradient (0-1%) of aqueous NH₃/(0-4%) MeOH/DCM, to give compound 77 (147 mg, 91%) as a red solid, mp (DCM/hexane) 119-122° C.; ¹H NMR δ 11.02 (br s, 1H, NH), 8.97 (dd, J=7.2, 1.5 Hz, 1H, ArH), 8.37 (dd, J=8.5, 1.4 Hz, 1H, ArH), 8.22 (br s, 1H, NH), 8.10 (d, J=8.7 Hz, 1H, ArH), 8.05 (s, 1H, ArH), 8.02 (d, J=8.9 Hz, 1H, ArH), 7.96 (dd, J=8.7, 7.2 Hz, 1H, ArH), 7.78 (dd, J=8.6, 6.9 Hz, 1H, ArH), 7.69-7.73 (m, 1H, ArH) 7.55 (dd, J=8.9, 1.6 Hz, 1H, ArH), 3.77 (br q, J=6.6 Hz, 2H, CH₂), 3.65 (br q, J=6.0 Hz, 2H, CH₂), 2.93 (s, 3H, CH₃), 2.61 (t, J=7.3 Hz, 2H, CH₂), 2.58 (t, J=6.1 Hz, 2H, CH₂), 2.49 (s, 3H, CH₃), 2.31 (s, 3H, CH₃), 2.07 (br quin, J=7.2 Hz, 2H, CH₂), 1.86 (br quin, J=6.2 Hz, 2H, CH₂); ¹³C NMR δ 165.1, 149.4, 143.2, 143.1, 141.0, 139.7, 137.8, 137.7, 136.8, 136.6, 135.1, 133.3, 131.0, 130.9, 130.0, 129.9, 129.4, 127.7, 120.1, 116.9, 56.4, 55.8, 42.0, 41.2, 38.3, 27.9, 25.7, 21.4, 18.1; HRMS (FAB⁺) calcd for C₂₉H₃₃N₈O₃ (MH⁺) m/z 541.2676, found 541.2669. Anal. calcd for C₂₉H₃₂N₈O₃.¼H₂O: C, 63.9; H, 6.0; N, 20.6; found: C, 63.8; H, 5.9; N, 20.8%.

### Example AA

### N-[3-(Methyl{3-[(6-methyl-1,4-dioxido-1,2,4-benzotriazin-3-yl)amino]propyl}amino)propyl]-4-acridinecarboxamide (85)

6-Methyl-1,2,4-benzotriazin-3-ol 1-Oxide (79). NaNO₂ (2.5 g, 36.3 mmol) was added in small portions to a stirred solution of 6-methyl-1,2,4-benzotriazin-3-amine 1-oxide (78) [Hay et. al., J. Med Chem. 2003, 46, 169-182] (3.2 g, 18.2 mmol) in TFA (15 mL) at −5 to 0° C. After the addition was completed the reaction mixture was stirred for further 1 h and poured into ice (150 g). The resulting pale yellow precipitate was filtered, washed with water and dried to give compound 79 (3.2 g, 97%), which was used without further purification.

3-Chloro-6-methyl-1,2,4-benzotriazine 1-Oxide (80). Compound 79 (3.2 g, 17.8 mmol) was heated at reflux in POCl₃ (25 mL) for 3 h. Excess reagent was evaporated and the residue was stirred in ice/water (150 mL) for 20 min. The resulting precipitate was filtered, air dried and purified by chromatography, eluting with a gradient (50-100%) of DCM/pet. ether, to give chloride 80 (2.5 g, 79%) as a white crystalline solid, mp (DCM/hexane) 156-158° C.; ¹H NMR δ 8.30 (d, J=8.8 Hz, 1H, H-8), 7.75 (br s, 1H, H-5), 7.64 (dd, J=9.4, 1.6 Hz, 1H, H-7), 2.62 (s, 3H, CH₃). Anal. calcd for C₈H₆ClN₃O: C, 49.1; H, 3.1; N, 21.5; found: C, 49.2; H, 3.1; N, 21.5%.

tert-Butyl 3-(Methyl{3-[(6-methyl-1-oxido-1,2,4-benzotriazin-3-yl)amino]propyl}amino)propylcarbamate (81). A mixture of chloride 80 (2.23 g, 11.4 mmol), tert-butyl-3-[(aminopropyl)(methyl)amino]propylcarbamate 69 (Huang et al., J. Med. Chem. 1992, 35, 2414-18) (3.34 g, 14.4 mmol) and triethylamine (2.3 mL, 16.5 mmol) in DME (60 mL) was heated at 85° C. for 3 h. The solvent was evaporated, the residue was dissolved in DCM (100 mL) and washed with aqueous NH₃ (40 mL). The organic layer was separated, the aqueous layer further extracted with DCM (3×30 mL), the combined organic fraction dried and the solvent evaporated. The residue was purified by chromatography, eluting with a gradient (0-1%) of aqueous NH₃/(0-5%) MeOH/DCM to give carbamate 81 (3.7 g, 80%) as a yellow solid, mp (DCM/hexane) 117-120° C.; ¹H NMR [(CD₃)₂SO] δ 8.01 (d, J=8.7 Hz, 1H, H-8), 7.84 (br s, 1H, NH), 7.37 (br s, 1H, H-5), 7.15 (dd, J=8.8, 1.7 Hz, 1H, H-7), 6.75 (t, J=5.2 Hz, 1H, NH), 3.33-3.37 (m, 2H, CH₂), 2.94 (br q, J=6.5 Hz, 2H, CH₂), 2.42 (s, 3H, CH₃), 2.35 (t, J=6.9 Hz, 2H, CH₂), 2.28 (t, J=7.0 Hz, 2H, CH₂), 2.08 (s, 3H, CH₃), 1.70 (br quin, J=6.9 Hz, 2H, CH₂), 1.51 (br quin, J=6.9 Hz, 2H, CH₂), 1.35 (s, 9H, 3×CH₃); ¹³C NMR [(CD₃)₂SO] 159.0, 155.5, 148.5, 146.6, 128.2, 126.4, 124.8, 119.5, 77.2, 54.8, 54.7, 41.6, 39.0, 38.2, 28.1 (3), 27.1, 26.1, 21.3; HRMS (FAB⁺) calcd for C₂₀H₃₃N₆O₃ (MH⁺) m/z 405.2614, found 405.2616.

N¹-(3-Aminopropyl)-N¹-methyl-N³-(6-methyl-1-oxido-1,2,4-benzotriazin-3-yl)-1,3-propanediamine (82). Carbamate 81 (2.1 g, 5.19 mmol) was dissolved in methanolic HCl (50 mL) and stirred 48 h at 20° C. Excess reagent and solvent were evaporated and the residue partitioned between DCM and aqueous NH₃. The organic layer was separated and the aqueous layer was further extracted with DCM (4×30 mL). The combined organic fraction was dried and the solvent evaporated. The residue was purified by chromatography, eluting with a gradient (0-1%) of aqueous NH₃/(3-7%) MeOH/DCM, to give amine 82 (1.57 g, 99%) as a yellow solid, mp 118-122° C. (DCM/MeOH); ¹H NMR [(CD₃)₂SO] δ 8.01 (d, J=8.8 Hz, 1H, H-8), 7.87 (br s, 1H, NH), 7.37 (br s, 1H, H-5), 7.16 (dd, J=8.8, 1.7 Hz, 1H, H-7), 3.14-3.34 (m, 4H, CH₂, NH₂), 2.54 (t, J=6.5 Hz, 2H, CH₂), 2.42 (s, 3H, CH₃), 2.35 (t, J=6.9 Hz, 2H, CH₂), 2.31 (t, J=7.2 Hz, 2H, CH₂), 2.13 (s, 3H, CH₃), 1.71 (br quin, J=7.0 Hz, 2H, CH₂), 1.47 (br quin, J=7.0 Hz, 2H, CH₂); HRMS (FAB⁺) calcd for C₁₅H₂₅N₆O (MH⁺) m/z 305.2090, found 305.2088.

2,2,2-Trifluoro-N-[3-(methyl{3-[(6-methyl-1-oxido-1,2,4-benzotriazin-3-yl)amino]propyl}amino)propyl]acetamide (83). CF₃CO₂Et (0.88 mL, 7.4 mmol) and H₂O (0.13 ml, 7.4 mmol) were added to a stirred solution of amine 82 (1.5 g, 4.9 mmol) in CH₃CN (50 mL) and the reaction mixture heated at reflux for 20 h. The solvent was evaporated and the residue partitioned between DCM and aqueous NaHCO₃. The organic layer was separated, the aqueous layer further extracted with DCM (3×30 mL), the combined organic fraction dried, and the solvent evaporated to give acetamide 83 (1.9 g, 100%) as a yellow solid, mp (DCM/hexane) 127-130° C.; ¹H NMR [(CD₃)₂SO] δ 9.43 (br s, 1H, NH), 8.01 (d, J=8.8 Hz, 1H, H-8), 7.85 (br s, 1H, NH), 7.36 (br s, 1H, H-5), 7.15 (dd, J=8.8, 1.5 Hz, 1H, H-7), 3.23 (br q, J=6.5 Hz, 2H, CH₂), 2.43 (s, 3H, CH₃), 2.38 (t, J=6.9 Hz, 2H, CH₂), 2.32, (t, J=6.9 Hz, 2H, CH₂), 2.15 (s, 3H, CH₃), 1.72 (br quin, J=7.0 Hz, 2H, CH₂), 1.64 (br quin, J=7.0 Hz, 2H, CH₂), CH₂ not observed; ¹³C NMR [(CD₃)₂SO] δ 159.0, 156.0 (q, J=36 Hz), 148.5, 146.6, 128.2, 126.4, 124.8, 119.5, 115.9 (q, J=288 Hz), 54.7, 54.5, 41.5, 38.9, 37.7, 26.1, 25.8, 21.3; HRMS (FAB⁺) calcd for C₁₇H₂₃F₃N₆O₂ (MH⁺) 401.1913, found 401.1896. Anal. calcd for C₁₇H₂₃F₃N₆O₂: C, 51.0; H, 5.8; F, 14.2; N, 21.0; found: C, 51.1; 6.0; F, 14.2; N, 21.0%.

2,2,2-Trifluoro-N-[3-(methyl{3-[(6-methyl-1,4-dioxido-1,2,4-benzotriazin-3-yl)amino]propyl}amino)propyl]acetamide (84). A solution of trifluoroperacetic acid [prepared from trifluoroacetic anhydride (5.7 mL, 6.3 mmol) and 70% H₂O₂ (2.0 mL, 6.3 mmol) in DCM (10 mL)] was added to a suspension of acetamide 83 (1.63 g, 4.1 mmol) and trifluoroacetic acid (0.63 mL, 8.1 mol) in DCM (20 mL) and the mixture stirred at 20° C. for 18 h. The mixture was slowly added to a cooled solution of aqueous NaHCO₃ (100 mL). The organic layer was separated and the aqueous layer extracted further with DCM (4×30 mL). The combined organic fraction was dried, and the solvent evaporated. The residue was purified by chromatography, eluting with a gradient (0-5%) of MeOH/DCM, to give (i) staring material 83 (807 mg, 49%) and (ii) dioxide 84 (509 mg, 30%) as a red solid, mp (DCM/hexane) 128-131° C.; ¹H NMR [(CD₃)₂SO] δ 9.43 (br s, 1H, NH), 8.42 (t, J=6.0 Hz, 1H, NH), 8.32 (d, J=8.9 Hz, 1H, H-8), 7.93 (s, 1H, H-5), 7.38 (dd, J=9.0, 1.7 Hz, 1H, H-7), 3.43 (br q, J=6.6 Hz, 2H, CH₂), 3.22 (br q, J=6.6 Hz, 2H, CH₂), 2.53 (s, 3H, CH₃), 9.38 (t, J=6.7 Hz, 2H, CH₂), 2.31 (t, J=6.9 Hz, 2H, CH₂), 2.15 (s, 3H, CH₃), 1.75 (br quin, J=6.9 Hz, 2H, CH₂), 1.65 (br quin, J=7.0 Hz, 2H, CH₂); ¹³C NMR [(CD₃)₂SO] δ 155.9 (q, J=36 Hz), 149.8, 146.9, 138 0, 128.8, 128.2, 120.8, 116.1 (q, J=288 Hz), 115.5, 54.9, 54.6, 41.5, 39.5, 37.7, 26.0, 25.8, 21.6; HRMS (FAB⁺) calcd for C₁₇H₂₄F₃N₆O₃ (MH⁺) m/z 417.1862, found 417.1868. Anal. calcd for C₁₇H₂₃F₃N₆O₃: C, 49.0; H, 5.6; F, 13.7; N, 20.2; found: C, 49.3; H, 5.9; F, 14.0; N, 20.4%.

N-[3-(Methyl{3-[(6-methyl-1,4-dioxido-1,2,4-benzotriazin-3-yl)amino]propyl}amino)propyl]-4-acridinecarboxamide (85). Aqueous NH₃ (5 mL) was added to a solution of dioxide 84 (125 mg, 0.3 mmol) in MeOH (5 mL) and the reaction mixture was stirred at 20° C. for 18 h. The solvent was evaporated, the residue was dissolved in DMF (5 mL), 4-(1H-imidazol-1-ylcarbonyl)acridine (164 mg, 0.6 mmol) was added and the mixture stirred at 20° C. for 48 h. The solvent was evaporated and the residue was purified by chromatography, eluting with a gradient (0-1%) of aqueous NH₃/(0-5%) MeOH/DCM, to give compound 85 (148 mg, 94%) as a red solid, mp (DCM/hexane) 158-160° C.; ¹H NMR [(CD₃)₂SO] δ 11.30 (t, J=5.4 Hz, 1H, NH), 9.23 (s, 1H, ArH), 8.67 (dd, J=7.1, 1.4 Hz, 1H, ArH), 8.48 (t, J=5.7 Hz, 1H, NH), 8.32 (dd, J=8.4, 1.2 Hz, 1H, ArH), 8.23 (d, J=8.7 Hz, 1H, ArH), 8.17 (d, J=8.4 Hz, 1H, ArH), 8.00 (d, J=8.9 Hz, 1H, ArH), 7.92 (ddd, J=7.7, 7.3, 1.4 Hz, 1H, ArH), 7.76 (s, 1H, ArH), 7.71 (t, J=7.7 Hz, 1H, ArH), 7.65 (t, J=7.4 Hz, 1H, ArH), 7.31 (dd, J=9.0, 1.5 Hz, 1H, ArH), 3.59 (br q, J=6.3 Hz, 2H, CH₂), 3.42 (br q, J=6.4 Hz, 2H, CH₂), 2.57 (t, J=7.0 Hz, 2H, CH₂), 2.47 (t, J=6.6 Hz, 2H, CH₂), 2.44 (s, 3H, CH₃), 2.23 (s, 3H, CH₃), 1.92 (br quin, J=6.9 Hz, 2H, CH₂), 1.79 (br quin, J=6.6 Hz, 2H, CH₂); ¹³C NMR [(CD₃)₂SO] δ 164.7, 149.6, 146.9, 146.8, 145.4, 138.4, 137.7, 134.3, 132.6, 131.8, 128.7, 128.6, 128.4, 128.3, 128.0, 126.4, 126.3, 125.5, 125.1, 120.7, 115.2, 55.4, 55.1, 41.7, 39.8, 37.4, 27.0, 25.8, 21.5; HRMS (FAB⁺) calcd for C₂₉H₃₂N₇O₃ (MH⁺) m/z 526.2567, found 526.2535. Anal. calcd for C₂₉H₃₁N₇O₃: C, 66.3; H, 5.9; N, 18.7; found: C, 66.0; H, 6.0; N, 18.8%.

### Example AB

N-[3-(Methyl{3-[(6-methyl-1,4-dioxido-1,2,4-benzotriazin-3-yl)amino]propyl}amino)propyl]-2-(4-pyridinyl)-8-quinolinecarboxamide (86). Aqueous NH₃ (5 mL) was added to a solution of dioxide 84 (126 mg, 0.3 mmol) in MeOH (5 mL) and the mixture stirred at 20° C. for 18 h. The solvent was evaporated, the residue dissolved in DMF (5 mL), 8-(1H-imidazol-1-ylcarbonyl)-2-(4-pyridinyl)quinoline (150 mg, 0.6 mmol) was added and the mixture stirred at 20° C. for 48 h. The solvent was evaporated, the residue dissolved in DCM (20 mL) and washed with water (3×15 mL). The organic layer was separated, dried, and the solvent evaporated. The residue was purified by chromatography, eluting with a gradient (0-1%) of aqueous NH₃/(0-3%) MeOH/DCM, to give compound 86 (165 mg, 100%) as a red solid, mp (DCM/hexane) 178-180° C.; ¹H NMR δ 10.81 (br s, 1H, NH), 8.77-8.83 (m, 3H, ArH), 8.40 (br s, 1H, NH), 8.32 (d, J=8.6 Hz, 1H, ArH), 8.13 (d, J=8.9 Hz, 1H, ArH), 7.87-7.93 (m, 5H, ArH), 7.65 (t, J=7.7 Hz, 1H, ArH), 7.21 (d, J=8.8 Hz, 1H, ArH), 3.72 (br q, J=6.3 Hz, 2H, CH₂), 3.49 (br q, J=5.3 Hz, 2H, CH₂), 2.54 (t, J=7.2 Hz, 2H, CH₂), 2.47 (s, 3H, CH₃), 2.46 (t, J=6.2 Hz, 2H, CH₂), 2.24 (s, 3H, CH₃), 2.01 (br quin, J=5.5 Hz, 2H, CH₂), 1.72 (br quin J=6.1 Hz, 2H, CH₂); ¹³C NMR δ 165.9, 154.4 (2), 150.8, 149.8, 147.6, 146.2, 145.3, 138.8, 138.1, 134.1, 131.1, 130.3, 128.8, 128.5, 127.8, 127.2, 121.7 (2), 121.3, 118.5, 116.0, 56.7, 55.7, 41.9, 41.3, 38.2, 27.7, 25.5, 22.2; HRMS (FAB⁺) calcd for C₃₀H₃₂N₈O₃ (MH⁺) m/z 553.2676, found 553.2673. Anal. calcd for C₃₀H₃₂N₈O₃.½H₂O: C, 64.2; H, 5.9; N, 20.0; found: C, 64.4; H, 6.1; N, 19.5%.

### Example AC

N-[3-(Methyl{3-[(6-methyl-1,4-dioxido-1,2,4-benzotriazin-3-yl)amino]propyl}amino)propyl]-1-phenazinecarboxamide (87). Aqueous NH₃ (6 mL) was added to a solution of dioxide 84 (145 mg, 0.35 mmol) in MeOH (10 mL) and stirred at 20° C. for 18 h. The solvent was evaporated, the residue dissolved in DMF (5 mL), 1-(1H-imidazol-1-ylcarbonyl)phenazine (183 mg, 0.68 mmol) was added and the mixture stirred at 20° C. for 48 h. The solvent was evaporated and the residue was purified by chromatography, eluting with a gradient (0-1%) of aqueous NH₃/(0-4%) MeOH/DCM, to give compound 87 (181 mg, 98%) as a red solid, mp (DCM/hexane) 111-114° C.; ¹H NMR δ 10.82 (br s, 1H, NH), 8.91 (dd, J=7.2, 1.5 Hz, 1H, ArH), 8.59 (br s, 1H, NH), 8.33 (dd, J=8.6, 1.5 Hz, 1H, ArH), 8.21-8.25 (m, 2H, ArH), 8.13 (d, J=8.9 Hz, 1H, ArH), 7.93 (dd, J=8.7, 7.1 Hz, 1H, ArH), 7.85-7.90 (m, 2H, ArH), 7.83 (s, 1H, ArH), 7.22 (dd, J=9.0, 1.7 Hz, 1H, ArH), 3.78 (br q, J=6.4 Hz, 2H, CH₂), 3.65 (br q, J=5.9 Hz, 2H, CH₂), 2.69 (t, J=7.3 Hz, 2H, CH₂), 2.63 (J=6.1 Hz, 2H, CH₂), 2.45 (s, 3H, CH₃), 2.36 (s, 3H, CH₃), 2.10 (br quin, J=7.1 Hz, 2H, CH₂), 1.89 (br quin, J=6.2 Hz, 2H, CH₂); ¹³C NMR δ 165.1, 149.8, 147.8, 143.3, 142.9, 141.4, 140.8, 138.0, 135.0, 134.9, 133.3, 131.6, 131.0, 130.0 (2), 129.0, 129.7, 129.0, 121.3, 115.7, 56.9, 55.8, 42.0, 41.6, 38.2, 27.5, 25.5, 22.2; HRMS (FAB⁺) calcd for C₂₈H₃₀N₈O₃ (MH⁺) m/z 527.2519, found 527.2506. Anal. calcd for C₂₈H₃₀N₈O₃.¼H₂O: C, 63.3; H, 5.8; N, 21.1; found: C, 63.3; H, 5.8; N, 21.5%.

### Example AD

9-Methyl-N-[3-({3-[(6-methyl-1,4-dioxido-1,2,4-benzotriazin-3-yl)amino]propyl}amino)propyl]-1-phenazinecarboxamide (88). Aqueous NH₃ (5 mL) was added to a solution of dioxide 84 (126 mg, 0.3 mmol) in MeOH (5 mL) and the reaction mixture was stirred at 20° C. for 18 h. The solvent was evaporated, the residue dissolved in DMF (5 mL), 1-(1H-imidazol-1-ylcarbonyl)-9-methylphenazine (172 mg, 0.6 mmol) was added and the mixture stirred at 20° C. for 48 h. The solvent was evaporated and the residue was purified by chromatography, eluting with a gradient (0-1%) of aqueous NH₃/(0-4%) MeOH/DCM, to give compound 88 (147 mg, 91%) as a red solid, mp (DCM/hexane) 80-83° C.; ¹H NMR δ 10.99 (t, J=5.2 Hz, 1H, NH), 8.96 (dd, J=7.1, 1.5 Hz, 1H, ArH), 8.43 (br s, 1H, NH), 8.36 (dd, J=8.5, 1.0 Hz, 1H, ArH), 8.14 (d, J=8.9 Hz, 1H, ArH), 8.10 (dd, J=8.8, 1.2 Hz, 1H, ArH), 7.96 (dd, J=8.8, 7.1 Hz, 1H, ArH), 7.96 (dd, J=8.7, 6.8 Hz, 1H, ArH), 7.77 (dd, J=8.7, 6.8 Hz, 1H, ArH), 7.69-7.73 (m, 1H, ArH), 7.22 (dd, J=8.9, 1.8 Hz, 1H, ArH), 3.77 (br q, J=6.6 Hz, 2H, CH₂), 3.66 (br q, J=6.1 Hz, 2H, CH₂), 2.93 (s, 3H, CH₃), 2.62 (t, J=7.2 Hz, 2H, CH₂), 2.58 (t, J=6.0 Hz, 2H, CH₂), 2.48 (s, 3H, CH₃), 2.32 (s, 3H, CH₃), 2.08 (br quin, J=7.2 Hz, 2H, CH₂), 1.86 (br quin, J=6.2 Hz, 2H, CH₂); ¹³C NMR δ 165.2, 149.8, 147.8, 143.1 (2), 141.0, 139.7, 138.1, 136.6, 135.0, 133.3, 131.0, 130.9, 130.0, 129.5, 129.0, 128.5, 127.7, 121.3, 115.8, 56.4, 55.8, 42.0, 41.3, 38.3, 27.9, 25.7, 22.3, 18.1; HRMS (FAB⁺) calcd for C₂₉H₃₃N₈O₃ (MH⁺) m/z 541.2676, found 541.2668

### Example AE

### N-{2-[{2-[(1,4-Dioxido-1,2,4-benzotriazin-3-yl)amino]ethyl}(methyl)amino]ethyl}-4-acridinecarboxamide (95)

tert-Butyl 2-[(2-Aminoethyl)(methyl)amino]ethylcarbamate (90). A solution of (BOC)₂O (9.60 g, 44 mmol) in THE (50 mL) was added over a period of 2 h to a solution of bis(diethylamino)methylamine (89) (10.32 g, 88 mmol) in THF (50 mL) at 0° C. The reaction mixture stirred for 30 min then allowed to warm to 20° C. and stirred for 20 h. The reaction mixture was partitioned between DCM and saturated aqueous NaCl, the organic layer separated and the aqueous layer further extracted with DCM (3×25 mL). The combined organic extract was dried and the solvent evaporated at 30° C. to give carbamate 90 (8.79 g, 46%) as a colourless oil, which was used without further purification.

tert-Butyl 2-(Methyl{2-[(1-oxido-1,2,4-benzotriazin-3-yl)amino]ethyl}amino)ethylcarbamate (91). A solution of chloride 3 (2.0 g, 11.0 mmol), carbamate 90 (2.9 g, 13.3 mmol) and triethylamine (3.0 mL, 22.1 mmol) in DME (50 mL) was heated at 85° C. for 3 h. The solvent was evaporated and the residue was partitioned between DCM (100 mL) and aqueous NH₃ (50 mL). The DCM layer was separated, the aqueous layer further extracted with DCM (3×30 mL), the combined organic fraction dried and the solvent evaporated. The residue was purified by chromatography, eluting with a gradient (0-1%) of aqueous NH₃/(0-5%) MeOH/DCM, to give (i) starting material 3 (500 mg, 25%) and (ii) carbamate 91 (2.1 g, 52%) as a yellow solid, mp (DCM/hexane) 122-124° C.; ¹H NMR [(CD₃)₂SO] δ 8.13 (dd, J=8.6, 1.0 Hz, 1H, H-8), 7.78 (ddd, J=8.4, 7.0, 1.6 Hz, 1H, H-6), 7.71 (br s, 1H, NH), 7.52 (d, J=8.2 Hz, 1H, H-5), 7.33 (ddd, J=7.8, 7.1, 1.2 Hz, 1H, H-7), 6.61 (br s, 1H, NH), 3.43 (br q, J=6.0 Hz, 2H, CH₂), 3.01 (br q, J=6.2 Hz, 9H, CH₂), 2.57 (t, J=6.6 Hz, 2H, CH₂), 2.42 (t, J=6.7 Hz, 2H, CH₂), 2.23 (s, 3H, CH₃), 1.35 (s, 9H, 3×CH₃); ¹³C NMR [(CD₃)₂SO] δ 158.8, 155.4, 148.2, 135.6, 129.9, 126.0, 124.4, 119.8, 77.4, 56.4, 55.5, 41.8, 38.5, 37.8, 28.1 (3); HRMS (FAB⁺) calcd for C₁₇H₂₇N₆O₃ (MH⁺) m/z 363.2145, found 363.2144. Anal. calcd for C₁₇H₂₆N₆O₃: C, 56.3; H, 7.2; N, 23.2; found: C, 56.5; H, 7.3; N, 23.3%.

N¹-(2-Aminoethyl)-N¹-methyl-N²-(1-oxido-1,2,4-benzotriazin-3-yl)-1,2-ethanediamine (92). Carbamate 91 (2.14 g, 5.9 mmol) was dissolved in methanolic HCl (30 mL) and stirred 20 h at 20° C. Excess reagent and solvent were evaporated and the residue was partitioned between DCM and aqueous NH₃. The organic fraction was separated and the aqueous faction was further extracted with DCM (4×30 mL). The combined organic fraction was dried and the solvent evaporated to give amine 92 (1.55 g, 100%) as yellow solid which was used without further purification, ¹H NMR [(CD₃)₂SO] δ 8.13 (dd, J=8.6, 1.3 Hz, 1H, H-8), 7.78 (ddd, J=7.7, 7.0, 1.4 Hz, 2H, H-6, NH), 7.57 (d, J=8.4 Hz, 1H, H-5), 7.33 (ddd, J=7.8, 7.1, 1.3 Hz, 1H, H-7), 3.42-3.68 (m, 2H, CH₂), 3.20-3.40 (m, 2H, CH₂), 2.58 (q, J=6.9 Hz, 2H, CH₂), 2.37 (t, J=6.5 Hz, 2H, CH₂), 2.22 (s, 3H, CH₃), 1.42 (br s, 2H, NH₂).

2,2,2-Trifluoro-N-[2-(methyl{2-[(1-oxido-1,2,4-benzotriazin-3-yl)amino]ethyl}amino)ethyl]acetamide (93). CF₃CO₂Et (2.05 mL, 17.2 mmol) and H₂O (0.31 ml, 17.2 mmol) was added to a solution of amine 92 (1.5 g, 5.7 mmol) in CH₃CN (50 mL) and the reaction mixture was heated at reflux for 48 h. The reaction mixture was evaporated and residue partitioned between DCM and aqueous NaHCO₃. The DCM layer was separated and the aqueous layer was further extracted with DCM (5×30 mL). The combined organic fraction was dried and the solvent evaporated to give trifluoroacetamide 93 (1.80 g, 88%) as a yellow solid, mp (DCM/hexane) 141-143° C.; ¹H NMR [(CD₃)SO] δ 9.29 (br s, 1H, NH), 8.13 (dd, J=8.6, 1.3 Hz, 1H, H-8), 7.78 (ddd, J=8.4, 7.0, 1.5 Hz, 1H, H-6), 7.68 (br s, 1H, NH), 7.57 (d, J=8.4 Hz, 1H, H-5), 7.33 (ddd, J=8.5, 7.1, 1.3 Hz, 1H, H-7), 3.42 (br q, J=6.3 Hz, 2H, CH₂), 3.30 (br q, J=6.8 Hz, 2H, CH₂), 2.61 (J=6.7 Hz, 2H, CH₂), 2.56(t, J=6.7 Hz, 2 H, CH₂), 2.27 (s, 3H, CH₃); ¹³C NMR [(CD₃)₂SO] δ 158.8, 156.1 (q, J=36 Hz) 148.3, 135.6, 130.0, 125.9, 124.4, 119.8, 115.8 (q, J=288 Hz), 55.4, 55.1, 41.7, 38.4, 37.2; HRMS (FAB⁺) calcd for C₁₄H₁₈F₃N₆O₂ (MH⁺) m/z 359.1443, found 359.1451. Anal. calcd for C₁₄H₁₇F₃N₆O₂: C, 46.9; H, 4.8; N, 23.5; F, 15.9; found: C, 47.2; H, 4.9; N, 3.6; F, 15.8%.

N-{2-[{2-[(1,4-Dioxido-1,2,4-benzotriazin-3-yl)amino]ethyl}(methyl)amino]ethyl}-2,2,2-trifluoroacetamide (94). A solution of trifluoroperacetic acid [prepared from trifluoroacetic anhydride (6.8 mL, 49 mmol) and 70% H₂O₂ (2.0 mL, 49 mmol) in DCM (20 mL)] was added to a solution of trifluoroacetamide 93 (1.75 g, 4.9 mmol) and trifluoroacetic acid (0.8 mL, 9.8 mmol) in DCM (20 mL) and the reaction mixture was sired at 20° C. for 5 h. The reaction mixture was slowly added to a cooled solution of aqueous NaHCO₃ (100 mL). The DCM layer was separated and the aqueous layer was further extracted with DCM (5×30 mL). The combined organic fraction was dried and the solvent was evaporated. The residue was purified by chromatography, eluting with a gradient (0-4%) of DCM/MeOH, to give (i) starting material 93 (100 mg, 6%); and (ii) dioxide 94 (859 mg, 47%) as a red solid, mp (DCM/hexane) 141-144° C.; ¹H NMR [(CD₃)₂SO] δ 9.28 (br s, 1H, NH), 8.20 (d, J=9.1 Hz, 1H, H-5), 8.12 (d, J=8.6 Hz, 1H, H-8), 8.03 (t, J=5.8 Hz, 1H, NH), 7.96 (ddd, J=8.6, 7.2, 1.3 Hz, 1H, H-6), 7.56 (ddd, J=8.6, 7.1, 1.3 Hz, 1H, H-7), 3.48 (br q, J=6.3 Hz, 2H, CH₂), 3.31 (br q, J=6.3 Hz, 2H, CH₂), 2.63, (t, J=6.6 Hz, 2H, CH₂), 2.54 (t, J=6.8 Hz, 2H, CH₂), 2.27 (s, 3H, CH₃); ¹³C NMR [(CD₃)₂SO] δ 156.1 (q, J=36 Hz), 149.7, 138.0, 135.4, 129.9, 126.9, 121.0, 115.8 (q, J=288 Hz), 116.7, 55.4, 55.0, 41.6, 38.3, 37.1; HRMS (FAB⁺) calcd for C₁₄H₁₈F₃N₆O₃ (MH⁺) m/z 375.1393, found 375.1392. Anal. calcd for C₁₄H₁₇F₃N₆O₃: C, 44.9; H, 4.6; N, 22.5; found: C, 44.8; H, 4.6; N, 22.5%.

N-{2-[{2-[(1,4-Dioxido-1,2,4-benzotriazin-3-yl)amino]ethyl}(methyl)amino]ethyl}-4-acridinecarboxamide (95). Aqueous NH₃ (6 mL) was added to a solution of dioxide 94 (125 mg, 0.33 mmol) in MeOH (6 mL) and the reaction mixture was stirred at 20° C. for 16 h. The solvent was evaporated, the residue dissolved in THF (5 mL), 4-(1H-imidazol-1-ylcarbonyl)acridine (180 mg, 0.66 mmol) was added and the mixture stirred at 20° C. for 48, h. The solvent was evaporated, the residue was dissolved in DCM (20 mL) and washed with water (3×15 mL). The organic fraction was dried and the solvent evaporated. The residue was purified by chromatography, eluting with a gradient (0-1%) of aqueous NH₃/(0-3%) MeOH/DCM, to give compound 95 (132 mg, 94%) as a red solid, mp (DCM/hexane) 160-162° C.; ¹H NMR δ 11.90 (br s, 1H, NH), 8.95 (dd, J=7.1, 1.5 Hz, 1H, ArH), 8.72 (s, 1H, ArH), 8.16 (d, J=8.6 Hz, 1H, ArH), 8.12 (d, J=8.8 Hz, 1H, ArH), 8.07 (dd, J=8.4, 1.4 Hz, 1H, ArH), 7.82-7.86 (m, 2H, ArH), 7.78 (ddd, J=7.7, 6.7, 1.5 Hz, 1H, ArH), 7.70 (ddd, J=7.8, 7.1, 1.3 Hz, 1H, ArH), 7.64 (dd, J=8.2, 7.1 Hz, 1H, ArH), 7.46 (ddd, J=7.5, 7.2, 0.7 Hz, 1H, ArH), 7.40 (ddd, J=7.9, 7.1, 1.3 Hz, 1H, ArH), 7.29 (br s, 1H, NH), 3.86 (br q, J=6.0 Hz, 2H, CH₂), 3.70 (br q, J=5.6 Hz, 2H, CH₂), 2.93 (t, J=6.4 Hz, 2H, CH₂), 2.89 (t, J=6.1 Hz, 2H, CH₂), 2.57 (s, 3H, CH₃); ¹³C NMR δ 166.0, 149.7, 147.4, 146.5, 137.3, 137.4, 135.3, 135.0, 132.1, 131.1, 130.1, 128.8, 128.6, 127.9, 126.8, 126.7, 126.1, 125.8, 125.5, 121.5, 117.2, 56.5, 55.9, 42.5, 39.1, 37.8; HRMS (FAB⁺) calcd for C₂₆H₂₆N₇O₃ (MH⁺) m/z 484.2097, found 484.2102.

### Example AF

N-{2-[{2-[(1,4-Dioxido-1,2,4-benzotriazin-3-yl)amino]ethyl}(methyl)amino]ethyl}-2-(4-pyridinyl)-8-quinolinecarboxamide (96). Aqueous NH₃ (6 mL) was added to a solution of dioxide 94 (132 mg, 0.35 mmol) in MeOH (10 mL) and the reaction mixture was stirred at 20° C. for 18 h. The solvent was evaporated, the residue dissolved in DMF (5 mL), 8-(1H-imidazol-1-ylcarbonyl)-2-(4-pyridinyl)quinoline (150 mg, 0.6 mmol) was added and the mixture stirred at 20° C. for 48 h. The solvent was evaporated, the residue was dissolved in DCM (20 mL) and washed with water (3×15 mL). The organic layer was separated, dried and the solvent evaporated. The residue was purified by chromatography, eluting with a gradient (0-1%) of aqueous NH₃/(0-3%) MeOH/DCM to give compound 96 (174 mg, 97%) as a red solid, mp (DCM/hexane) 130-135° C.; ¹H NMR δ 11.00 (t, J=5.0 Hz, 1H, NH), 8.83 (dd, J=5.3, 2.2 Hz, 1H, ArH), 8.81 (d, J=7.7 Hz, 1H, ArH), 8.81 (dd, J=4.7, 3.0 Hz, 1H, ArH), 8.29 (d, J=8.6 Hz, 1H, ArH), 8.23 (dd, J=8.1 Hz, 1H, ArH), 7.98 (d, J=9.7 Hz, 1H, ArH), 7.92-7.93 (m, 2H, ArH), 7.87-7.90 (m, 2H, ArH), 7.76 (ddd, J=6.1, 5.4, 2.2 Hz, 1H, ArH), 7.65 (dd, J=7.4, 6.5 Hz, 1H, ArH), 7.44 (ddd, J=7.9, 7.0, 1.3 Hz, 1H, ArH), 7.21 (br, 1H, NH), 3.79 (br q, J=6.1 Hz, 2H, CH₂), 3.48 (br q, J=5.8 Hz, 2H, CH₂), 2.82 (t, J=6.3 Hz, 2H, CH₂), 2.73 (t, J=6.1 Hz, 2H, CH₂), 2.43 (s, 3H, CH₃); ¹³C NMR δ 165.8, 154.6 (2), 150.7, 149.6, 146.4, 145.4, 138.8, 138.0, 135.2, 134.5, 131.3, 130.2, 129.5, 127.9, 127.1, 126.9, 121.8 (2) 121.6, 118.7, 117.3, 56.8, 56.1, 42.3, 38.8, 37.9; HRMS (FAB⁺) calcd for C₂₇H₂₇N₈O₃ (MH⁺) m/z 511.2206, found 511.2208. Anal. calcd for C₂₇H₂₆N₈O₃.¼H₂O: C, 63.0; H, 5.2; N, 21.8; found: C, 63.0; H, 5.2; N, 21.5%.

### Example AG

N-{2-[{2-[(1,4-Dioxido-1,2,4-benzotriazin-3-yl)amino]ethyl}(methyl)amino]ethyl}-1-phenazinecarboxamide (97). Aqueous NH₃ (6 mL) was added to a solution of dioxide 94 (120 mg, 0.32 mmol) in MeOH (10 mL) and reaction mixture was stirred at 20° C. for 18 h. The solvent was evaporated, the residue dissolved in DMF (5 mL), and 1-(1H-imidazol-1-ylcarbonyl)phenazine (172 mg, 0.64 mmol) added and the mixture stirred at 20° C. for 48 h. The solvent was evaporated and the residue was purified by chromatography, eluting with a gradient (0-1%) of aqueous NH₃/(0-3%) MeOH/DCM, to give compound 97 (137 mg, 88%) as a red solid, mp (DCM/hexane) 163-165° C.; ¹H NMR δ 11.07 (br s, 1H, NH), 8.95 (dd, J=7.1, 1.5 Hz, 1H, ArH), 8.31 (dd, J=8.7, 1.5 Hz, 1H, ArH), 8.07-8.11 (m, 2H, ArH), 8.03 (dd, J=8.7, 0.7 Hz, 1H, ArH), 7.93 (dd, J=8.7, 7.1 Hz, 1H, ArH), 7.78 (ddd, J=7.7, 6.8, 1.6, 1H, ArH), 7.65-7.72 (m, 2H, ArH), 7.60 (dd, J=8.6, 0.9 Hz, 1H, ArH), 7.38 (ddd, J=7.8, 7.0, 1.5 Hz, 1H, ArH), 7.19 (br s, 1H, NH), 3.85 (br q, J=5.8 Hz, 2H, CH₂), 3.68 (br q, J=5.6 Hz, 2H, CH₂), 2.85-2.91 (m, 4H, 2×CH₂), 2.57 (s, 3H, CH₃); ¹³C NMR δ 165.0, 149.6, 143.4, 142.7, 141.3, 141.0, 137.6, 135.2 (2), 133.4, 131.2, 130.5, 130.0, 129.9, 129.6, 129.5, 128.8, 126.9, 121.3, 117.0, 56.2, 55.8, 42.3, 38.9, 37.8; HRMS (FAB⁺) calcd for C₂₅H₂₅N₈O₃ (MH⁺) m/z 485.2050, found 485.2045. Anal calcd for C₂₅H₂₄N₈O₃: C, 62.0; H, 5.0; N, 23.1; found: C, 61.7; H, 4.7; N, 23.1%.

### Example AH

N-{2-[{2-[(1,4-Dioxido-1,2,4-benzotriazin-3-yl)amino]ethyl}(methyl)amino]ethyl}-9-methyl-1-phenazinecarboxamide (98). Aqueous NH₃ (6 mL) was added to a solution of dioxide 94 (120 mg, 0.32 mmol) in MeOH (10 mL) and the reaction mixture was stirred at 20° C. for 16 h. The solvent was evaporated, the residue dissolved in THF (5 mL), and 1-(1H-imidazol-1-ylcarbonyl)-9-methylphenazine (185 mg, 0.6 mmol) was added and the mixture stirred at 20° C. for 48 h. The solvent was evaporated and the residue was purified by chromatography, eluting with a gradient (0-1%) of aqueous NH₃/(0-3%) MeOH/DCM, to give compound 98 (128 mg, 80%) as a red solid, mp (DCM/hexane) 161-163° C.; ¹H NMR δ 11.03 (br s, 1H, NH), 8.94 (dd, J=7.1, 1.5 Hz, 1H, ArH), 8.29 (dd, J=8.6, 1.5 Hz, 1H, ArH), 8.12 (dt, J=8.8, 0.7 Hz, 1H, ArH), 7.94-7.98 (m, 1H, ArH), 7.91 (dd, J=8.6, 7.1 Hz, 1H, ArH), 7.60-7.70 (m, 4H, ArH), 7.78 (ddd, J=8.3, 7.3, 1.1 Hz, 1H, ArH), 7.26 (br s, 1H, NH), 3.86 (br q, J=6.1 Hz, 2H, CH₂), 3.60 (br q, J=5.5 Hz, 2H, CH₂), 2.91 (s, 3H, CH₃), 2.87 (t, J=6.2 Hz, 2H, CH₂), 2.80 (t, J=5.9 Hz, 2H, CH₂), 2.47 (s, 3H, CH₃); ¹³C NMR δ 165.2, 149.5, 143.1, 141.0, 140.0, 137.6, 136.7, 135.2, 135.1, 133.3, 130.7, 130.6, 129.9 (2), 129.4, 127.6, 126.8, 121.3, 117.0, 56.6, 55.8, 42.3, 38.8, 37.7, 17.9, one resonance not observed; HRMS (FAB⁺) calcd for C₂₆H₂₇N₈O₃ (MH⁺) m/z 499.2206, found 499.2200. Anal. calcd for C₂₆H₂₆N₈O₃: C, 62.6, H; 5.3; N, 22.5; found: C, 62.2; H, 5.3; N, 22.4%.

### Example AI

N-{2-[{2-[(1,4-Dioxido-1,2,4-benzotriazin-3-yl)amino]ethyl}(methyl)amino]ethyl}-5-methyl-4-acridinecarboxamide (99). Aqueous NH₃ (6 mL) was added to a solution of dioxide 94 (125 mg, 0.33 mmol) in MeOH (10 mL) and the reaction mixture was stirred at 20° C. for 24 h. The solvent was evaporated, the residue dissolved in THF (5 mL) and 4-(1H-imidazol-1-ylcarbonyl)-5-methylacridine (208 mg, 0.72 mmol) was added and the mixture stirred at 20° C. for 24 h. The solvent was evaporated, the residue dissolved in DCM (20 mL) and washed with water (3×15 mL). The organic layer was dried and the solvent evaporated. The residue was purified by chromatography, eluting with a gradient (0-1%) of aqueous NH₃/(0-4%) MeOH/DCM, to give compound 99 (180 mg, 100%) as a red solid, mp (DCM/hexane) 148-152° C.; ¹H NMR δ 11.90 (br s, 1H, NH), 8.94 (dd, J=7.1, 1.5 Hz, 1H, ArH), 8.70 (s, 1H, ArH), 8.17 (d, J=8.2 Hz, 1H, ArH), 8.05 (dd, J=8.3, 1.4 Hz, 1H, ArH), 7.88 (d, J=8.2 Hz, 1H, ArH), 7.96 (d, J=7.9, Hz, 1H, ArH), 7.69-7.73 (m, 1H, ArH), 7.60-7.65 (m, 2H, ArH) 7.38-7.43 (m, 2H, ArH), 7.33 (br s, 1H, NH), 3.85 (br q, J=6.3 Hz, 2H, CH₂), 3.61 (br q, J=4.2 Hz, 2H, CH₂), 2.90 (s, 3H, CH₃), 2.89 (t, J=6.7 Hz, 2H, CH₂), 2.56 (t, J=6.0 Hz, 2H, CH₂), 2.47 (s, 3H, CH₃); ¹³C NMR δ 166.2, 149.7, 147.0, 145.4, 137.9, 137.8, 135.9, 135.2, 135.1, 132.1, 130.8, 130.1, 128.4, 126.7, 126.6, 126.2, 126.1, 125.8, 125.4, 121.5, 117.2, 56.9, 55.8, 42.3, 39.0, 37.9, 18.8; HRMS (FAB⁺) calcd for C₂₇H₂₈N₇O₃ (MH⁺) m/z 498.2254, found 498.2257. Anal. calcd for C₂₇H₂₇N₇O₃.¼H₂O: C, 64.6; H, 5.5; N, 19.5; found: C, 64.5; H, 5.5; N, 19.7%.

### Example AJ

N-{3-[{3-[(1,4-Dioxido-1,2,4-benzotriazin-3-yl)amino]propyl}(methyl)amino]propyl}-1-phenazinecarboxamide (100). Aqueous NH₃ (6 mL) was added to a solution of trifluoroacetamide 39 (283 mg, 0.70 mmol) in MeOH (10 mL) and the reaction mixture was stirred at 20° C. for 18 h. The solvent was evaporated, the residue dissolved in DMF (5 mL), 1-(1H-imidazol-1-ylcarbonyl)phenazine (283 mg, 1.05 mmol) added and the mixture stirred at 20° C. for 48 h. The solvent was evaporated and the residue was purified by chromatography, eluting with a gradient (0-1%) of aqueous NH₃/(0-3%) MeOH/DCM, to give compound 100 (293 mg, 82%) as a red solid, mp (DCM/hexane) 129-130° C.; ¹H NMR δ 10.85, (br s, 1H, NH), 8.93 (dd, J=7.1, 1.4 Hz, 1H, ArH), 8.52 (br, 1H, NH), 8.33 (dd, J=8.7, 1.4 Hz, 1H, ArH), 8.21-8.27 (m, 2H, ArH), 8.11 (d, J=8.7 Hz, 1H, ArH), 7.93 (dd, J=8.6, 6.5 Hz, 1H, ArH), 7.86-7.90 (m, 3H, ArH), 7.70 (t, J=7.8 Hz, 1H, ArH), 7.42 (t, J=7.8 Hz, 1H, ArH), 3.77 (br q, J=6.4 Hz, 2H, CH₂), 3.66 (br q, J=5.7 Hz, 2H, CH₂), 2.68 (t, J=7.3 Hz, 2H, CH₂), 2.62 (t, J=6.1 Hz, 2H, CH₂), 2.36 (s, 3H, CH₃), 2.10 (br quin, J=7.1 Hz, 2H, CH₂), 1.89 (br quin, J=6.2 Hz, 2H, CH₂); ¹³C NMR δ 165.0, 149.8, 143.4, 142.9, 141.4, 140.8, 138.2, 135.4, 135.1, 135.0, 133.4, 131.5, 130.9, 130.1 (2), 129.8, 129.0, 126.7, 121.6, 117.1, 56.7, 55.9, 42.1, 41.5, 38.2, 27.5, 25.6; HRMS (FAB⁺) calcd for C₂₇H₂₉N₈O₃ (M⁺) m/z 513.2363, found 513.2365. Anal. calcd for C₂₇H₂₈N₈O₃: C, 54.8; H, 6.0; N, 31.9; found: C, 55.1; H, 5.8; N, 32.3%.

### Example AK

N¹-(2-aminoethyl)-N-(1,4-dioxido-1,2,4-benzotriazin-3-yl)-1,2-ethanediamine (101). A solution of carbamate (36) (252 mg, 0.54 mmol) in methanolic HCl was stirred at 20° C. for 24 h. Excess reagent and solvent were evaporated and the residue partitioned between aqueous NH₃ and DCM. The organic layer was separated and the aqueous layer was extracted with DCM (15×20 mL). The combined organic extract was dried, and the solvent evaporated to give amine 101 (109 mg, 76%) as a gum which was used without further purification, ¹H NMR δ 8.33 (d, J=8.7 Hz, 1H, ArH), 8.30 (d, J=8.8 Hz, 1H, ArH), 7.87 (ddd, J=8.5, 7.1, 1.0 Hz, 1H, ArH), 7.50 (ddd, J=8.4, 7.1, 1.2 Hz, 1H, ArH), 3.70 (br t, J=5.9 Hz, 2H, CH₂), 2.98 (br t, J=5.9H, 2H, CH₂), 2.84 (br t, J=5.6 Hz, 2H, CH₂), 2.74 (br t, J=5.6 Hz, 2H, CH₂), 2×NH, NH₂ not observed.

N-[2-({2-[(1,4-Dioxido-1,2,4-benzotriazin-3-yl)amino]ethyl}amino)ethyl]-2-(4-pyridinyl)-8-quinolinecarboxamide (102). 8-(1H-Imidazol-1-ylcarbonyl)-2-(4-pyridinyl)quinoline (198 mg, 0.78 mmol) was added to a solution of amine (101) (105 mg, 0.39 mmol) in DMF (10 mL) and the reaction mixture was stirred at 20° C. for 16 h. The solvent was evaporated and the residue purified by chromatography, eluting with a gradient (0-2%) of aqueous NH₃/(0-4%) MeOH/DCM to give compound 102 (148 mg, 75%) as a red solid, mp (DCM/hexane) 160-165° C.; ¹H NMR [(CD₃)₂SO] δ 10.62 (t, J=5.4 Hz, 1H, NH), 8.75 (d, J=6.1 Hz, 1H, ArH), 8.75 (dd, J=4.5, 1.8 Hz, 1H, ArH), 8.68 (d, J=8.7 Hz, 1H, ArH), 8.57 (dd, J=7.3, 1.6 Hz, 1H, ArH), 8.28 (d, J=8.7 Hz, 1H, ArH), 8.21 (dd, J=8.2, 1.6 Hz, 1H, ArH), 8.18 (d, J=6.2 Hz, 1H, ArH), 8.15 (dd, J=3.5, 1.7 Hz, 1H, ArH), 8.11 (br s, 1H, NH), 8.07 (dd, J=8.6, 0.9 Hz, 1H, ArH), 7.98 (dd, J=8.8, 0.7 Hz, 1H, ArH), 7.88 (ddd, J=7.8, 7.0, 1.4 Hz, 1H, ArH), 7.77 (dd, J=8.0, 7.4 Hz, 1H, ArH), 7.51 (ddd, J=7.8, 7.0, 1.5 Hz, 1H, ArH), 3.61 (br q, J=5.8 Hz, 2H, CH₂), 3.41-3.45 (m, 2H, CH₂), 2.90 (t, J=5.9 Hz, 2H, CH₂), 2.87 (t, J=6.2 Hz, 2H, CH₂), NH not observed; HRMS (FAB⁺) calcd for C₂₆H₂₅N₇O₃ (MH⁺) m/z 497.2050, found 497.2058. Anal. calcd for C₂₆H₂₄N₈O₃: C, 62.9; H, 4.9; N, 22.7; found: C, 62.9, H, 4.9; N, 22.7.

### Example AL

N-[2-({2-[(1,4-Dioxido-1,2,4-benzotriazin-3-yl)amino]ethyl}amino)ethyl]-5-methyl-4-acridinecarboxamide (103). 4-(1H-Imidazol-1-ylcarbonyl)-5-methylacridine (245 mg, 0.86 mmol) was added to a solution of amine 101 (113 mg, 0.43 mmol) in DMF (5 mL) and the reaction mixture stirred at 20° C. for 16 h. The solvent was evaporated and the residue purified by chromatography, eluting with a gradient (0-2%) of aqueous NH₃/(0-4%) MeOH/DCM, to give compound 103 (156 mg, 75%) as a red solid, mp (DCM/hexane) 135-140° C.; ¹H NMR [(CD₃)₂SO] δ 11.52 (t, J=5.5 Hz, 1H, CONH), 9.25 (s, 1H, ArH), 8.74 (dd, J=7.1, 1.6 Hz, 1H, ArH), 8.36 (dd, J=8.6, 1.5 Hz, 1H, ArH), 8.18 (br, 1H, NH), 8.10 (dd, J=8.6, 0.9 Hz, 1H, ArH), 8.03 (d, J=8.4 Hz, 1H, ArH), 7.96 (dd, J=8.7, 1.2 Hz, 1H, ArH), 7.90 (ddd, J=7.8, 5.9, 1.4 Hz, 1H, ArH), 7.73-7.77 (m, 2H, ArH), 7.49-7.57 (m, 2H, ArH), 3.65 (br q, J=6.0 Hz, 2H, CH₂), 3.47-3.51 (m, 2H, CH₂), 2.92 (t, J=6.0 Hz, 2H, CH₂), 2.88 (s, 3H, CH₃), 2.86 (t, J=6.3 Hz, 2H, CH₂), NH not observed; ¹³C NMR [(CD₃)₂SO] δ 164.8, 149.8, 146.4, 144.6, 138.7, 137.8, 135.4, 135.2, 134.5, 132.6, 131.1, 129.7, 128.2, 126.7, 126.4, 126.2 (2), 125.5, 125.1, 120.9, 116.6, 48.4, 47.8, 40.4, 39.6, 18.2; HRMS (FAB⁺) calcd for C₂₆H₂₆N₇O₃ (MH⁺) m/z 484.2097, found 484.2094.

### Example AM

### N-{5-[4-(Dimethylamino)butanoyl]-1-methyl-1H-pyrrol-3-yl}-4-({4-[(1,4-dioxido-1,2,4-benzotriazin-3-yl)amino]butanoyl}amino)-1-methyl-1H-pyrrole-2-carboxamide (112)

Methyl 4-[({4-[(tert-Butoxycarbonyl)amino]-1-methyl-1H-pyrrol-2-yl}carbonyl)amino]-1-methyl-1H-pyrrole-2-carboxylate (106). A solution of methyl 4-amino-1-methyl-1H-pyrrole-2-carboxylate (104) (Baird & Dervan, J. Am. Chem. Soc. 1996, 118, 6141-6146) (792 mg, 4.2 mmol) and 4-[(tert-butoxycarbonyl)amino]-1-methyl-1H-pyrrole-2-carboxylic acid (105) (Baird & Dervan, J. Am. Chem. Soc. 1996, 118, 6141-6146) (1.0 g, 4.2 mmol) in DMF (13 mL) and DCM (3 mL) was treated with EDCI (1.5 g, 6.2 mmol) and DMAP (0.77 g, 5.0 mmol). The reaction mixture was stirred at 20° C. for 18 h, poured into 10% HCl (20 mL) and extracted with EtOAc (3×40 mL). The combined organic fraction was washed with saturated aqueous NaHCO₃, dried and the solvent evaporated. The residue was purified by chromatography, eluting with a gradient (0-50%) of EtOAc/pet. ether to give amide 106 (1.0 g, 64%) as a white solid, mp (DCM/pet. ether) 89-90° C.; ¹H NMR [(CD₃)₂SO] δ 9.82 (s, 1H, NH), 9.06 (br s, 1H, NH), 7.44 (d, J=1.9 Hz, 1H, ArH), 6.90 (d, J=2.0 Hz, 1H, ArH), 6.88 (br s, 1H, ArH), 6.83 (br s, 1H, ArH), 3.83 (s, 3H, CH₃), 3.80 (s, 3H, CH₃), 3.74 (s, 3H, CO₂CH₃), 1.46 (s, 9H, 3×CH₃); ¹³C NMR [(CD₃)₂SO] δ 168.7, 158.3, 152.7, 122.9, 122.5, 122.3, 120.6, 118.4, 117.1, 108.3, 103.8, 78.2, 50.8, 36.0, 35.9, 28.1 (3); HRMS (EI⁺) calcd for C₁₈H₂₄N₄O₅ (M⁺) m/z 376.1747, found 376.1744.

4-{[(4-Amino-1-methyl-1H-pyrrol-2-yl)carbonyl]amino}-1-methyl-1H-pyrrole-2-carboxylic Acid (107). A solution of LiOH (343 mg, 14.3 mmol) in water (7 mL) was added to a solution of amide 106 (1.0 g, 2.7 mmol) in THF/MeOH (3:1, 28 mL) and the mixture heated at 60° C. for 18 h. The mixture was cooled and diluted with EtOAc (150 mL). The aqueous layer was separated, adjusted to the pH 3 with 10% aqueous HCl and extracted with EtOAc (3×40 mL). The combined organic fraction was dried and the solvent evaporated to give acid 107 (900 mg, 94%) as a white solid, mp (DCM/hexane) 138-142° C.; ¹H NM [(CD₃)₂SO] δ 11.70 (br s, 1H, CO₂H), 9.78 (s, 1H, NH), 9.05 (s, 1H, NH), 7.38 (d, J=1.9 Hz, 1H, ArH), 6.90 (br s, 1H, ArH), 6.82 (d, J=2.0 Hz, 2H, ArH), 3.81 (s, 3H, CH₃), 3.80 (s, 3H, CH₃), 1.46 (s, 9H, 3×CH₃); ¹³C NMR [(CD₃)₂SO] δ 171.8, 161.8, 158.3, 152.8, 122.6, 122.3, 120.1, 119.4, 117.0, 108.3, 103.7, 78.2, 36.0, 35.9, 28.1 (3); HRMS (FAB⁺) calcd for C₁₇H₂₃N₄O₅ (MH⁺); m/z 363.1669, found 363.1653.

tert-Butyl 5-({[5-({[3-(Dimethylamino)propyl]amino}carbonyl)-1-methyl-1H-pyrrol-3-yl]amino}carbonyl)-1-methyl-1H-pyrrol-3-ylcarbamate (108). A solution of acid 107 (900 mg, 2.5 mmol) was treated with EDCI (950 mg, 5.0 mmol), DMAP (758 mg, 6.2 mmol) and 3-dimethylaminopropylamine (507 mg, 5.0 mmol). The reaction mixture was stirred at 20° C. for 18 h, diluted with EtOAc (100 ml) and washed with 10% aqueous HCl (3×20 mL). The combined aqueous fraction was basified with aqueous NH₃, extracted with EtOAc (3×40 mL), dried and the solvent evaporated. The residue was purified by chromatography, eluting with a gradient (0-1%) of aqueous NH₃/(0-5%) MeOH/DCM, to give amide 108 (847 mg, 76%) as a viscous oil, which solidified on standing, mp DCM/pet. ether) 120-123° C.; ¹H NMR [(CD₃)₂SO] δ 9.77 (s, 1H, NH), 9.04 (br s, 1H, NH), 8.01 (t, J=5.6 Hz, 1H, NH), 7.15 (d, J=1.8 Hz, 1H, ArH), 6.87 (br s, 1H, ArH), 6.81 (d, J=1.8 Hz, 9H, ArH), 3.80 (s, 3H, CH₃), 3.79 (s, 3H, CH₃), 3.18 (br q, J=6.5 Hz, 2H, CH₂), 2.24 (t, J=7.1 Hz, 2H, CH₂), 2.13 [s, 6H, N(CH₃)₂] 1.89 (br quin, J=7.0 Hz, 2H, CH₂), 1.46 (s, 9H, 3×CH₃).

Methyl 4-[(1-Oxido-1,2,4-benzotriazin-3-yl)amino]butanoate (109). A solution of chloride 3 (1.57 g, 8.7 mmol), methyl 4-aminobutanoate hydrochloride (1.73 g, 11.4 mmol) and Et₃N (3.14 mmol, 22.5 mmol) in DME (50 mL) was heated at 90° C. for 6 h. The solvent was evaporated and the residue was partitioned between DCM (100 mL) and water (50 mL). The organic fraction was separated and the aqueous layer was further extracted with DCM (4×30 mL). The combined organic fraction was dried, the solvent evaporated and the residue purified by chromatography, eluting with a gradient (0-2%) of MeOH/DCM, to give ester 109 (1.9 g, 81%) as a yellow solid, mp (DCM/pet. ether) 122-126° C.; ¹H NMR [(CD₃)₂SO] δ 8.11 (dd, J=8.6, 1.1 Hz, 1H, H-8), 7.90 (br s, 1H, NH), 7.76 (ddd, J=7.7, 7.1, 1.5 Hz, 1H, H-6), 7.54 br d, J=8.3 Hz, 1H, H-5), 7.31 (ddd, J=7.9, 7.0, 1.2 Hz, 1H, H-7), 3.58(s, 3H, OCH₃), 3.34-3.38 (m, 2H, CH₂), 2.41 (t, J=7.4 Hz, 2H CH₂), 1.86-1.91 (m, 2H, CH₂); ¹³C NMR [(CD₃)₂SO] δ 173.2, 159.1, 148.4, 138.2, 135.7, 126.1, 124.6, 120.0, 51.3, 40.0, 30.8, 24.0; HRMS (EI⁺) calcd for C₁₂H₁₄N₄O₃ (M⁺) m/z 262.1066, found 262.1066. Anal. calcd for C₁₂H₁₄N₄O₃: C, 55.0; H, 5.4; N, 21.4; found: C, 55.1; H, 5.4; N, 21.4%.

Methyl 4-[(1,4-Dioxido-1,2,4-benzotriazin-3-yl)amino]butanoate (110). Hydrogen peroxide (70%, 3.1 mL, 65 mmol) was added dropwise to a stirred solution of trifluoroacetic anhydride (9.0 mL, 65 mmol) in DCM (15 mL) at 0° C. and the solution stirred at 0° C. for 10 minutes. The solution was added to a solution of ester 109 (1.7 g, 6.5 mmol) in DCM (30 mL) at 20° C. and stirred for 16 h. The reaction mixture was poured into saturated aqueous NaHCO₃ (100 mL), the organic layer separated and the aqueous layer further extracted with DCM (3×30 mL). The combined organic fraction was dried and the solvent evaporated. The residue was purified by chromatography, eluting with a gradient (0-2%) of MeOH/DCM, to give (i) starting material 109 (610 mg, 36%); and (ii) 1,4-dioxide 110 (592 mg, 33%) as a red solid, mp (DCM/pet. ether) 169-171° C.; ¹H NMR [(CD₃)₂SO] δ 8.33 (t, J=4.1 Hz, 1H, NH), 8.20 (dd, J=8.8, 0.7 Hz, 1H, H-8), 8.12 (dd, J=8.7, 0.7 Hz, 1H, H-5), 7.93 (ddd, J=7.8, 7.1, 1.4 Hz, H-6), 7.56 (ddd, J=7.8, 7.1, 1.3 Hz, 1H, H-7), 3.59 (s, 3H, OCH₃), 3.42 (br q, J=6.6 Hz, 2H, CH₂), 2.40 (t, J=7.4 Hz, 2H, CH₂), 1.88 (br quin, J=7.1 Hz, 2H, CH₂); ¹³C NMR [(CD₃)₂SO] δ 173.0, 149.8, 138.2, 135.4, 129.9, 126.9, 121.1, 116.8, 51.2, 39.9, 30.5, 23.9; HRMS (EI⁺) calcd for C₁₂H₁₄N₄O₄ (M⁺) m/z 278.1015, found 278.1014. Anal. calcd for C₁₂H₁₄N₄O₄: C, 51.8; H, 5.1; N, 20.1; found: C, 51.6; H, 4.9; N, 20.1%.

4-[(1,4-Dioxido-1,2,4-benzotriazin-3-yl)amino]butanoic Acid (111). A mixture of 1,4-dioxide 110 (351 mg, 1.26 mmol) and 1 N NaOH (6.3 mL, 6.30 mmol) in MeOH (20 mL) was stirred at 20° C. for 18 h. 10% Aqueous HCl (7 mL) was added and MeOH was evaporated. The resulting red precipitate was filtered, washed with water and dried to give acid 111 (270 mg, 81%) yield, mp (H₂O) 185-188° C.; ¹H NMR [(CD₃)₂SO] δ 8.82 (br s, 1H, NH), 8.19 (dd, J=8.8, 0.6 Hz, 1H, H-8), 8.11 (dd, J=8.4, 0.9 Hz, 1H, H-5), 7.92 (ddd, J=7.8, 7.1, 1.4 Hz, 1H, H-6), 7.54 (ddd, J=7.8, 7.2, 1.3 Hz, 1H, H-7), 3.38 (t, J=6.9 Hz, 2H, CH₂), 1.99 (t, J=7.0 Hz, 1H, CH₂), 1.79 (br quin, J=7.0 Hz, 2H, CH₂); HRMS (EI) calcd for C₁₁H₁₂N₄O₄ (M⁺) m/z 264.0845, found 264.0850. Anal. calcd for C₁₁H₁₂N₄O₄: C, 50.0, H, 4.6, N, 21.2; found: C, 50.1; H, 4.5, N, 21.2%.

N-{5-[4-(Dimethylamino)butanoyl]-1-methyl-1H-pyrrol-3-yl}-4-({4-[(1,4-dioxido-1,2,4-benzotriazin-3-yl)amino]butanoyl}amino)-1-methyl-1-pyrrole-2-carboxamide (112). Carbamate 108 (166 mg, 0.37 mmol) was dissolved in HCl/MeOH (3 mL) and stirred for 16 h. The solvent was evaporated and the residue was dissolved in MeOH (5 mL) and evaporated. This process was repeated two more times. The residue was dissolved in DMF (5 mL) and DCM (2 mL) and acid 111 (264 mg, 0.38 mmol), EDCI (146 mg, 0.76 mmol) and DMAP (93 mg, 0.76=mol) were added and the mixture stirred for 16 h at 20° C. The solvent was evaporated and the residue was partitioned between DCM and aqueous NH₃. The resulting precipitate was collected by filtration and purified by chromatography, eluting with a gradient (0-1%) of aqueous NH₃/(0-5%) MeOH/DCM, to give compound 112 (21 mg, 9%) as an orange solid, mp (DCM/pet. ether) 140-145° C.; ¹H NMR [(CD₃)₂SO] δ 9.81 (s, 1H, ArH, 9.79 (s, 1H, ArH), 8.35 (t, J=6.1 Hz, 1H, NH), 8.21 (d, J=8.5 Hz, 1H, H-8), 8.14 (d, J=8.1 Hz, 1H, H-5), 8.03 (t, J=5.7 Hz, 1H, NH), 7.94 (ddd, J=7.8, 7.1, 0.8 Hz, 1H, H-6), 7.59 (ddd, J=7.9, 7.1, 1.3 Hz, 1H, H-7), 7.25 (d, J=1.8 Hz, 1H, ArH), 7.13 (d, J=1.8 Hz, 1H, ArH), 6.85 (d, J=1.8 Hz, 1H, ArH), 6.81 (d, J=1.8 Hz, 1H, ArH), 3.81 (s, 3H, CH₃), 3.79 (s, 3H, CH₃), 3.46 (br q, J=6.6 Hz, 2H, CH₂), 3.19 (br q, J=6.5 Hz, 2H, CH₂), 2.32 (br q, J=6.9 Hz, 4H, 2×CH₂), 2.19 [s, 6H, N(CH₃)₂], 1.93 (br quin, J=7.2 Hz, 2H, CH₂), 1.63 (br quin, J=7.0 Hz, 2H, CH₂), 3-NH not observed; ¹³C NMR [(CD₃)₂SO] δ 169.0, 161.1, 158.3, 149.7, 138.1, 135.3, 129.8, 126.8, 122.9, 122.6, 121.9, 121.8, 121.0, 118.0, 117.6, 116.8, 103.9 (2), 56.8, 44.9 (2), 40.5, 36.9, 35.9, 35.8, 32.9, 26.9, 25.0; HRMS (FAB⁺) calcd for C₂₈H₃₇N₁₀O₅ (MH⁺) m/z 593.2948, found 593.2953. Anal. calcd for C₂₈H₃₆N₁₀O₅.H₂O: C, 55.1; H, 6.3; N, 22.9; found: C, 55.1; H, 6.6, N, 22.2. %.

### Example AN

### tert-Butyl 2-[(3-Ethyl-1,4-dioxido-1,2,4-benzotriazin-7-yl)oxy]ethylcarbamate (117)

N-{2-[(3-Amino-1-oxido-1,2,4-benzotriazin-7-yl)oxy]ethyl}-2,2,2-trifluoroacetamide (113). A mixture of compound 46 (520 mg, 3.0 mmol), K₂CO₃ (833 mg, 6.0=mmol) and N-(2-bromoethyl)-2,2,9-trifluoroacetamide (1.25 g, 4.0 mmol) in DMF (20 mL) was stirred at 100° C. for 16 h. The solvent was evaporated and the residue suspended in water. The suspension was extracted with EtOAc (3×50 mL), the organic fraction dried and the solvent evaporated. The residue was purified by chromatography, eluting with 5% MeOH/DCM, to give compound 113 (639 mg, 66%) as a tan solid, mp (DCM/pet. ether) 234-236° C. Anal. calcd for C₁₁H₁₀F₃N₅O₃: C, 41.7; H, 3.2; N, 22.1; F, 18.0; found: C, 41.9; H, 3.0; N, 21.9; F, 17.5%.

N-{2-[(3-Chloro-1-oxido-1,2,4-benzotriazin-7-yl)oxy]ethyl}-2,2,2-trifluoroacetamide (114). A solution of NaNO₂ (652 mg, 9.5 mmol) in water (20 mL) was added dropwise to a stirred suspension of amine 113 (1.5 g, 4.7 mmol) in 2 M HCl (75 mL) at 0° C. and the mixture stirred at 20° for 16 h. The suspension was filtered, the solid washed with water (2×10 mL) and dried to give 2,2,2-trifluoro-N-{2-[(3-hydroxy-1-oxido-1,2,4-benzotriazin-7-yl)oxy]ethyl}acetamide (1.44 g, 100%) as a tan solid, mp 202-204° C. Anal. calcd for C₁₁HOF₃N₄O₄: C, 41.5; H, 2.9; N, 17.6; F, 17.9; found: C, 41.8; H, 2.9; N, 17.4; F, 17.6%.

A mixture of the 3-hydroxide (1.39 g, 4.1 mmol) and POCl₃ (15 mL) was stirred at 100° C. for 2 h. The solution was cooled and poured into ice/water and stirred for 30 min. The precipitate was filtered, washed with water, and dried. The solid was purified by chromatography, eluting with a gradient (0-10%) of EtOAc/DCM, to give chloride 114 (1.37 g, 100%) as a tan solid, mp 179-181° C. Anal. calcd for C₁₁H₈ClF₃N₄O₃: C, 39.2; H, 2.4; N, 16.6; F, 16.9; found: C, 39.5; H, 2.5; N, 16.7; F, 16.9%.

N-{2-[(3-Ethyl-1-oxido-1,2,4-benzotriazin-7-yl)oxy]ethyl}-2,2,2-trifluoroacetamide (115). Pd(PPh₃)₄ (198 mg, 0.17 mmol) was added to a purged solution of chloride 114 (1.16 g, 3.4 mmol) and Et₄Sn (0.82 mL, 4.1 mmol) in DME (50 mL) and the mixture heated at reflux temperature under N₂ for 16 h. The solvent was evaporated and the residue purified by chromatography, eluting with 1% MeOH/DCM, to give compound 115 (896 mg, 79%) as a cream solid, mp (DCM) 155-157° C. Anal. calcd for C₁₃H₁₃F₃N₄O₃: C, 47.3; H, 4.0; N, 17.0; found: C, 47.4; H, 4.2; N, 17.1%.

tert-Butyl 2-[(3-Ethyl-1-oxido-1,2,4-benzotriazin-7-yl)oxy]ethylcarbamate (116). A solution of compound 115 (370 mg, 1.1 mmol) in 0.5 M K₂CO₃ solution (15 mL), was stirred at 20° C. for 16 h. The solution was extracted with CHCl₃ (3×30 mL), the organic fraction dried and the solvent evaporated. The residue was dissolved in THF (50 mL) and di-tert-butyl dicarbonate (367 mg, 1.68 mmol) added and the solution stirred at 20° C. for 5 h. The solution was partitioned between EtOAc and water, the organic fraction dried and the solvent evaporated. The residue was purified by chromatography, eluting with 10% EtOAc/DCM, to give carbamate 116 (330 mg, 88%) as a white solid, mp. 101-103° C.

tert-Butyl 2-[(3-Ethyl-1,4-dioxido-1,2,4-benzotriazin-7-yl)oxy]ethylcarbamate (117). A mixture of 1-oxide 116 (300 mg, 0.9 mmol) and MCPBA (663 mg, 2.7 mmol) in DCM (20 mL) was stirred at 20° C. for 36 h. The solution was partitioned between dilute aqueous NH₃ and DCM, the organic fraction dried and the solvent evaporated. The residue was purified by chromatography, eluting with a gradient (0-20%) of EtOAc/DCM, to give 1,4-dioxide (239 mg, 76%) as a red powder, mp (DCM/pet. ether) 111-113° C.

### Example AO

### Cytotoxicity of Compounds

Evaluation of the Cytotoxicity of Compounds by Clonogenic Assay Under Aerobic and Hypoxic Conditions.

Compounds representative of the invention were evaluated under both aerobic and hypoxic conditions in clonogenic assays, using three cell lines: human colon carcinoma HT-29, murine SCCVII, and human lung adenocarcinoma LXFL. Clonogenic survival was determined using aerobic and hypoxic SCCVII cell suspensions. Drug exposures were performed using continuously stirred and gassed single cell suspensions (10⁶ cells/mL) at 37° C., equilibrated with 5% CO₂ in air or N₂ for 60 min before drug addition. After a 60 min drug exposure cells were washed by centrifugation and plated to determine colony formation Cytotoxicity was measured as the concentration required to reduce plating efficiency to 10% of controls (C₁₀). The hypoxic cytotoxicity ratio (HCR) was determined as the ratio of the C₁₀ values under aerobic and hypoxic conditions. The relative hypoxic toxicity (RHT) was determined as the ratio of hypoxic TPZ C₁₀ to hypoxic BTO C₁₀. The results of these assays are given in Table 2. Abbreviations used in Table 2 are:

C₁₀=The concentration of drug (in micromolar) to reduce viable cell numbers to 10% of those of control cell cultures grown under the same conditions but not exposed to drug

RHT=Relative hypoxic toxicity is defined as the ratio of concentrations of Tirapazamine/test compound to give equal cell killing under hypoxic conditions.

HCR=Hypoxic cytotoxicity ratio is defined as the ratio of drug concentrations under aerobic and hypoxic condition to produce equal cell survival (10%) determined by clonogenic assay

TABLE 2
Cytotoxicities of compounds of the invention under hypoxic
conditions, hypoxic toxicity relative to Tirapazamine (RHT)
and hypoxic selectivity (HCR) in clonogenic assay

| | | | |
|---|---|---|---|
| | C₁₀ hypoxic | | |
| compound | (μM) | RHT | HCR |

HT 29 cells

| | | | |
|---|---|---|---|
| 11 | 0.12 | 416 | 83.0 |
| 30 | 0.9 | 78 | 33.0 |

SCVIII cells

| | | | |
|---|---|---|---|
| | SN | | |
| | (hypoxic) | | |
| 11 | 0.48 | 16.7 | 20.0 |
| 17 | 4.8 | 1.94 | >6.3 |
| 30 | 0.3 | 20 | 21.3 |
| 41 | 0.8 | 12.5 | 52.5 |
| 44 | 0.16 | 56.3 | >187 |
| 43 | 1.4 | 5.7 | 10 |
| 45 | 0.31 | 29 | 23.9 |
| 55 | 1.1 | 10 | 63.6 |
| 95 | 1.0 | 11 | 400 |
| 96 | 2.3 | 3.9 | 65 |
| 99 | 0.29 | 17.2 | 176 |

LXFL cells

| | | | |
|---|---|---|---|
| 11 | 0.04 | 450 | 35.0 |
| 30 | 0.4 | 50 | 12.5 |
| 41 | 0.4 | 37.5 | 50 |

The result of Table 2 clearly show that the compounds of the invention show large increases in cytotoxicity compared with Tirapazamine, while retaining selective killing under hypoxic conditions.

### Example AP

### Cytotoxicity of Compounds

Evaluation of the Cytotoxicity of Compounds by Proliferation Assay (IC₅₀) Under Aerobic and Hypoxic Conditions.

Compounds representative of the invention were evaluated under both aerobic and hypoxic conditions in a proliferation assay (IC₅₀), using two cell lines: human colon carcinoma HT-29, and human cervical carcinoma SiHa.

Drug exposures were performed in 96-well plates (Nunc) using either a 37° C. humidified incubator (20% O₂, 5% CO₂) or in the incubator compartment (37° C.) of an anaerobic chamber (Shell Lab) where palladium catalyst scrubbed gas (90% N₂, 5% H₂, 5% CO₂) ensures severe anoxia (<0.001% O₂). For each experiment, compounds were simultaneously tested under both oxic and hypoxic conditions against the HT-29 cell line and included TPZ as an independent internal control at the front and back of the assay (n=2). Final data was pooled from a series of seven independent experiments and is calculated using inter-experimental means. In all cases, 8-methyl-5-nitroquinoline was used as a second internal control to confirm that strict hypoxia was present during the experiment. (Siim et al. Br. J. Cancer 1994, 70, 596-603). Cell cultures were grown in αMEM (Gibco) containing 5% heat inactivated FCS and maintained in exponential growth phase. For each individual experiment an appropriate number of cells were seeded (HT-29=1000) into wells in αMEM÷10% FCS+10 mM added glucose+100 μM 2′-deoxycytidine (2′dCyd), and allowed to attach for 3 h. High glucose (final concentration 17 mM) and the presence of 2′-dCyd minimize hypoxia-induced cell cycle arrest. Replicates were then exposed to BTOs, using 2-fold serial dilutions in triplicate, for a further 4 h. Subsequently cells were washed free of compound using complete media (without glucose/2′-dCyd) and allowed to grow for 5 (oxic) or 6 (anoxic) days. Plates were stained as described previously (Wilson et al., J. Med. Chem. 1989, 32, 31-38) and IC₅₀ values determined.

IC₅₀=The concentration of drug (in micromolar) to reduce viable cell numbers to 50% of those of control cell cultures grown under the same conditions but not exposed to drug

RHT=Relative hypoxic toxicity is defined as the ratio of concentrations of Tirapazamine/test compound to give equal cell killing under hypoxic conditions.

HCR=Hypoxic cytotoxicity ratio is defined as the ratio of drug concentrations under aerobic and hypoxic condition to produce equal cell survival (50%) determined by proliferation assay

TABLE 3
Cytotoxicities of compounds of the invention under hypoxic
conditions, hypoxic toxicity relative to Tirapazamine (RHT)
and hypoxic selectivity (HCR) in proliferation assay

| | | | |
|---|---|---|---|
| | IC₅₀ hypoxic | | |
| Compound | (μM) | RHT | HCR |

HT-29 IC₅₀

| | | | |
|---|---|---|---|
| 11 | 0.016 | 370 | 38 |
| 30 | 0.065 | 90 | 167 |
| 31 | 0.356 | 163 | 5.3 |
| 37 | 0.079 | 74 | 160 |
| 41 | 0.043 | 134 | 154 |
| 42 | 0.517 | 11.2 | 86.2 |
| 43 | 0.113 | 51.4 | 119 |
| 44 | 0.226 | 25.7 | 72.7 |
| 45 | 0.018 | 321 | 31 |
| 55 | 0.124 | 47 | 97 |
| 62 | 0.021 | 274 | 157 |
| 63 | 0.034 | 167 | 129 |
| 74 | 0.130 | 44.7 | 95 |
| 75 | 0.200 | 29 | 92 |
| 85 | 0.222 | 26 | 134 |
| 86 | 0.225 | 66 | 168 |
| 95 | 0.18 | 71 | 74 |
| 96 | 0.19 | 31 | 77 |
| 98 | 0.135 | 114 | 45 |
| 99 | 0.41 | 14 | 25 |
| 102 | 0.49 | 12 | 54 |
| 103 | 0.035 | 357 | 83 |

SiHa IC₅₀

| | | | |
|---|---|---|---|
| 30 | 0.031 | 121 | 41 |
| 55 | 0.05 | 72 | 124 |
| 75 | 0.07 | 53 | 110 |
| 86 | 0.105 | 35 | 136 |
| 95 | 0.076 | 48 | 89 |
| 96 | 0.10 | 37 | 78 |
| 98 | 0.075 | 63 | 30 |
| 102 | 0.16 | 23 | 53 |
| 103 | 0.01 | 309 | 118 |

The results of Table 3 clearly show that the compounds of the invention show large increases in cytotoxicity compared with Tirapazamine, while retaining selective killing under hypoxic conditions.

Wherein the foregoing description reference has been made to reagents, or integers having known equivalents thereof, then those equivalents are herein incorporated as if individually set forth.

While this invention has been described with reference to certain embodiments and examples, it is to be appreciated that further modifications and variations can be made to embodiments and examples without departing from the scope of the invention.

1. A compound of Formula I, 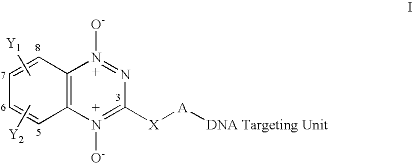
wherein
Y₁ and Y₂ at one or more of the available carbons 5-8 on the benzo ring: are each independently selected from the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
wherein each R is independently selected from an optionally substituted C₁₋₆ alicyclic or an optionally substituted C₃₋₆ cyclic alkyl group, and wherein the said optional substituents are each independently selected from; halo, OH, OR¹, NO₂ NH₂, NHR¹, NR¹R¹, SH, SR¹, imidazolyl, R¹-piperazinyl, morpholino, SO₂R¹, CF₃, CN. CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, HNR², NR²₂ or N(OH)R² wherein each R² is independently selected from C₁₋₄ alkyl, C₂₋₄ alkenyl, OH, NO₂, NH₂, CF₃, CN, CO₂H or SH, and
wherein X is selected from NH, NMe, CH₂, SO, SO₂, or O;
A is an optionally substituted C₁₋₁₂ alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂ NH₂, CF₃, CN, CO₂H or SH; and wherein the optionally substituted C₁₋₁₂ alkyl chain is optionally interrupted or extended by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C., or a pharmacologically acceptable salt thereof.

2. The compound of Formula I as claimed in claim 1 wherein the DNA-targeting unit is selected from one of formulae II-XVI, 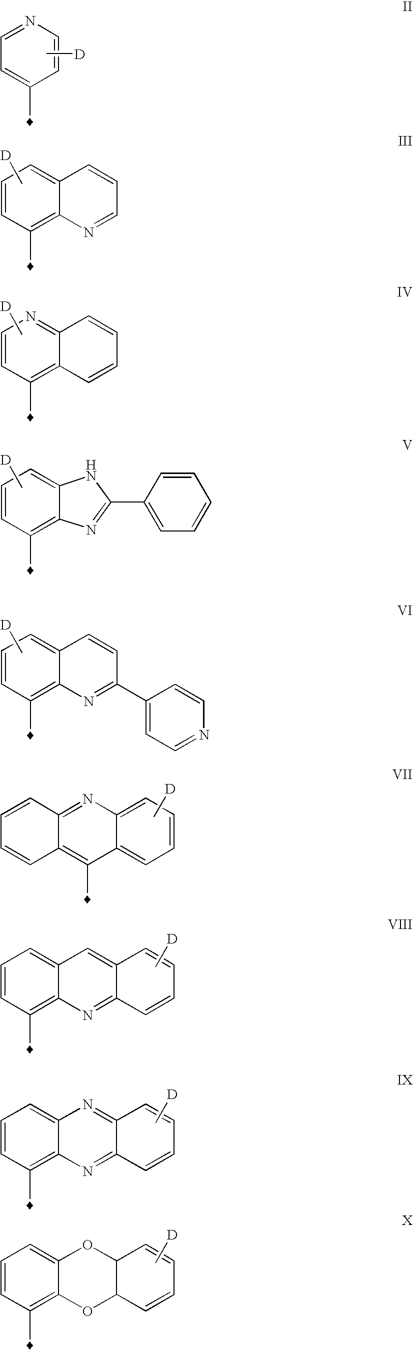 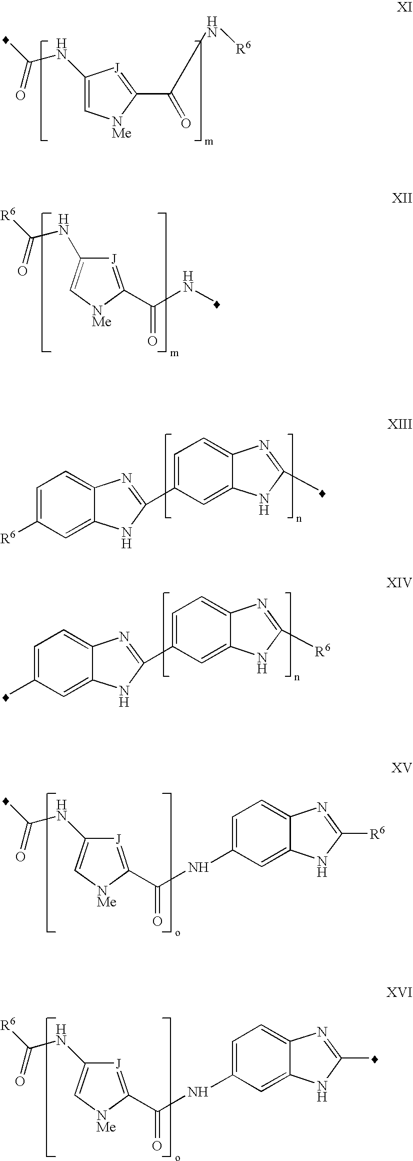
wherein in structures XI-XVI R⁶ is independently selected from an optionally substituted C₁₋₆ alicyclic or an optionally substituted C₃₋₆ cyclic alkyl group, and wherein the optional substituents are each independently selected from; halo, OH, OR⁷NO₂, NH₂, NHR⁷, NR⁷R⁷, SR⁷, imidazolyl, R piperazinyl, morpholino, SO₂R⁷, CF₃, CN, CO₂H, CO₂R⁷, CHO, COR⁷, CONH₂, CONHR⁷, CONR⁷R⁷;
R⁶ can also represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸, NR⁸₂ or N(OH)R⁸ wherein each R⁸ is independently selected from C₁₋₄ alkyl, C₂₋₄ alkenyl, OH, NO₂ NH₂, CF₃, CN, CO₂H or SH;
wherein D represents up to four of the following groups as substituents at any available ring carbon position; H, R⁹, hydroxy, alkoxy, halogen, NO₂, NH₂, NHR⁹, NR⁹₂, SH, SR⁹ SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl and morpholino, wherein each R⁹ is independently selected from an optionally substituted C₁₋₄ alkyl or an optionally substituted C₂₋₄ alkenyl group and wherein the optional substituents are each independently selected from OH, OR¹⁰, NH₂, NHR¹⁰NR¹⁰₂ or N(OH)R¹⁰ wherein each R¹⁰ is independently selected from C₁₋₄ alkyl, C₂₋₄ alkenyl, OH, NO₂, NH₂, CF₃, CN, CO₂H or SH; and wherein any available ring carbon position of formulae II-XVI is optionally replaced by —N— when the valency and configuration of the formula allows, the point of attachment of formulae II-XVI to the A group defined above is represented by ♦; and
wherein in formulae XI, XII, m is selected from 2, 3 or 4, and
wherein in formulae XI, XII, XV and XVI, J is selected from CH or N; and
wherein in formulae XIII and XIV n is selected from 0, 1 or 2; and wherein in formulae XV and XVI o is selected from 1 and 2.

3. The compound of Formula I as claimed in claim 2 wherein the DNA targeting unit is selected from one of formulae IV, V, VI, VII, VIII, or IX.

4. The compound of Formula I as claimed in claim 2 wherein D of the DNA targeting unit of Formulae II-X is H or Me.

5. The compound of Formula I as claimed in claim 1 wherein X is NH or CH₂.

6. The compound of Formula I as claimed in claim 1 wherein Y₁ and Y₂ each represent H.

7. The compound of Formula I as claimed in claim 1 wherein Y₁ represents OMe.

8. The compound of Formula I as claimed in claim 1 wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

9. The compound of Formula I as claimed in claim 2 wherein X is NH—, Y₁ is H, Y₂ is H, A is —(CH₂)₆NH—, the DNA targeting unit represents formula VII and D is H.

10. The compound of Formula I as claimed in claim 2 wherein X is NH—, Y₁ is H, Y₂ is H, A is —(CH₂)₃NH(CH₂)₃NHCO—, the DNA targeting unit represents formula VIII and D is H.

11. The compound of Formula I as claimed in claim 2 wherein X is NH—, Y₁ is H, Y₂ is H, A is —(CH₂)₂NH(CH₂)₂NHCO—, the DNA targeting unit represents formula VIII and D is H.

12. The compound of Formula I as claimed in claim 2 wherein X is NH—, Y₁, is H, Y₂ is H, A is —(CH₂)₃NMe(CH₂)₃NHCO—, the DNA targeting unit represents formula VIII and D is H.

13. The compound of Formula I as claimed in claim 2 wherein X is NH—, Y₁ is H, Y₂ is H, A is —(CH₂)₃NMe(CH₂)₃NHCO—, the DNA targeting unit represents formula IV and D is H.

14. The compound of Formula I as claimed in claim 2 wherein X is NH—, Y₁ is H, Y₂ is H, A is —(CH₂)₃NMe(CH₂)₃NHCO—, the DNA targeting unit represents formula VI and D is H.

15. The compound of Formula I as claimed in claim 2 wherein X is NH—, Y₁ is H, Y₂ is H, A is —(CH₂)₃NMe(CH₂)₃NHCO—, the DNA targeting unit represents formula VIII and D is Me.

16. The compound of Formula I as claimed in claim 2 wherein X is NH—, Y₁ is H, Y₂ is H, A is —(CH₂)₃NMe(CH₂)₃NHCO—, the DNA targeting unit represents formula IX and D is Me.

17. The compound of Formula I as claimed in claim 2 wherein X is NH—, Y₁ is 7-MeOCH₂CH₂O—, Y₂ is H, A is —(CH₂)₃NMe(CH₂)₃NHCO—, the DNA targeting unit represents formula VIII and D is H.

18. The compound of Formula I as claimed in claim 2 wherein X is CH₂—, Y₁ is H, Y₂ is H, A is —(CH₂)₂NMe(CH₂)₃NHCO—, the DNA targeting unit represents formula VIII and D is H.

19. The compound of Formula I as claimed in claim 2 wherein X is NH—, Y₁ is H, Y₂ is H, A is —(CH₂)₂NMe(CH₂)₃NHCO—, the DNA targeting unit represents formula XI and D is H.

20. The compound of Formula I as claimed in claim 2 wherein X is NH—, Y₁ is 7-Me, Y₂ is H, A is —(CH₂)₃NMeH(CH₂)₃NHCO—, the DNA targeting unit represents formula VIII and D is H.

21. The compound of Formula I as claimed in claim 2 wherein X is NH—, Y₁ is 7-Me, Y₂ is H, A is —(CH₂)₃NMe(CH₂)₃NHCO—, the DNA targeting unit represents formula VI and D is H.

22. The compound of Formula I as claimed in claim 2 wherein X is NH—, Y₁ is 6-Me, Y₂ is H, A is —(CH₂)₃NMe(CH₂)₃NHCO—, the DNA targeting unit represents formula VIII and D is H.

23. The compound of Formula I as claimed in claim 2 wherein X is NH—, Y₁ is 6-Me, Y₂ is H, A is —(CH₂)₃NMe(CH₂)₃NHCO—, the DNA targeting unit represents formula VI and D is H.

24. The compound of Formula I as claimed in claim 2 wherein X is NH—, Y₁ is H, Y₂ is H, A is —(CH₂)₂NMe(CH₂)₂NHCO—, the DNA targeting unit represents formula VIII and D is H.

25. The compound of Formula I as claimed in claim 2 wherein X is NH—, Y₁ is H, Y₂ is H, A is —(CH₂)₂NMe(CH₂)₂NHCO—, the DNA targeting unit represents formula VI and D is H.

26. The compound of Formula I as claimed in claim 2 wherein X is NH—, Y₁ is H, Y₂ is H, A is —(CH₂)₂NMe(CH₂)₂NHCO—, the DNA targeting unit represents formula XI and D is Me.

27. The compound of Formula I as claimed in claim 2 wherein X is NH—, Y₁ is H, Y₂ is H, A is —(CH₂)₂NMe(CH₂)₂NHCO—, the DNA targeting unit represents formula VIII and D is Me.

28. The compound of Formula I as claimed in claim 2 wherein X is NH—, Y₁ is H, Y₂ is H, A is —(CH₂)₂NH(CH₂)₂NHCO—, the DNA targeting unit represents formula VI and D is H.

29. The compound of Formula I as claimed in claim 2 wherein X is NH—, Y₁ is H, Y₂ is H, A is —(CH₂)₂NH(CH₂)₂NHCO—, the DNA targeting unit represents formula VIII and D is Me.

30. A compound of Formula I′, 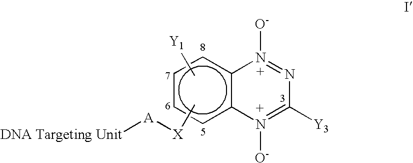
wherein
Y₁ represents at one or more of the available carbons 5-8 on the benzo ring the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
Y₃ is selected from the following groups halo, H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl and morpholino;
wherein each R of groups Y₁ and Y₃ is independently selected from an optionally substituted C₁₋₆ alicycic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted c₁₋₄ alkyl or an optionally substituted C₂₋₄ alkenyl group and wherein the optional substituents are each independently selected from OH, OR, NH₂, NHR²NR²₂ or N(OH)R² wherein each R² is independently selected from C₁₋₄ alkyl, C₂₋₄ alkenyl, OH, NO₂, NH₂, CF₃, CN, CO₂H or SH, and wherein X represents NH, NMe, CH₂, SO, SO₂, or O;
wherein A represents an optionally substituted C₁₋₂ alkyl group wherein the optional substituents are each independently selected from OH, OR, NH₂, NHR³, NR³₂ or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₂₋₁₂ alkyl chain is optionally interrupted by one or more heteroatom containing linkage moieties selected from O, NH, NR⁴, CONH, CONR⁴, NHCO, NR⁴CO, wherein each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; and
wherein the DNA-targeting unit is any moiety of a molecular weight below 700 Daltons that has an association constant (K) for binding to double-stranded random-sequence DNA of >10³ M⁻¹ at an ionic strength of 0.01 M at 20° C., or a pharmacologically acceptable salt thereof.

31. The compound of Formula I′ as claimed in claim 30 wherein the DNA-targeting unit is selected from one of formulae II-XVI, 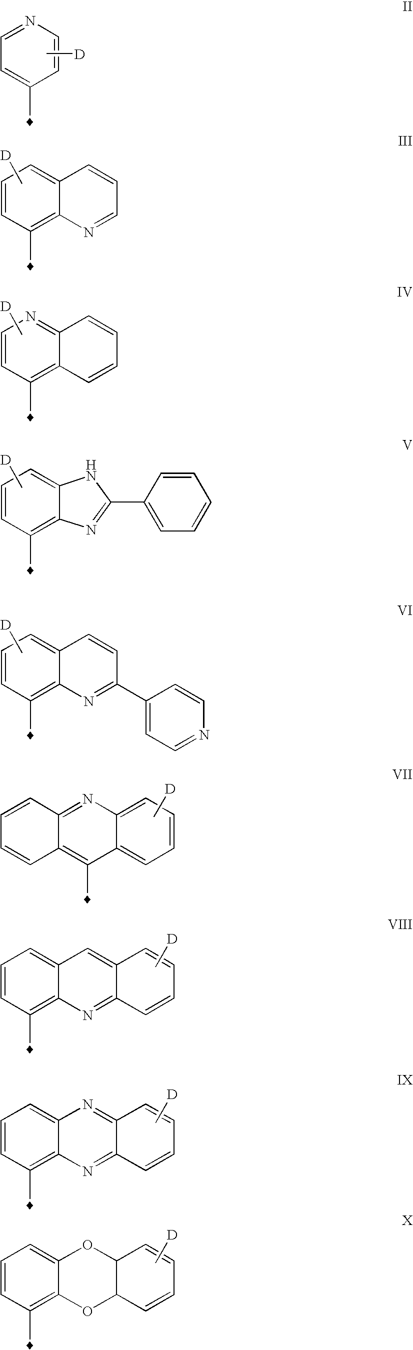 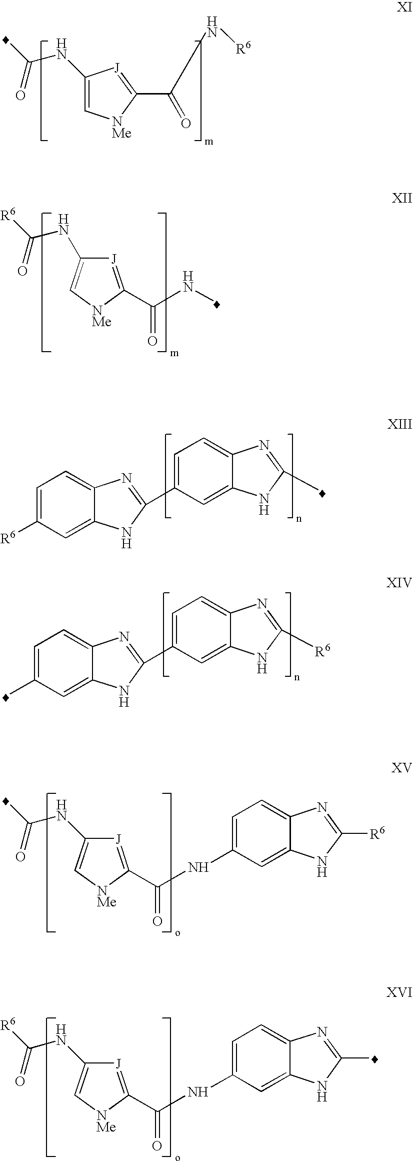
wherein in structures XI-XVI R⁶ is independently selected from an optionally substituted C₁₋₆ alicydic or an optionally substituted C₃₋₆ Cyclic alkyl group, and wherein the optional substituents are each independently selected from; halo, OH, OR⁷NO₂, NH₂, NHR⁷, NR⁷R⁷, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷;
R⁶ can also represent an optionally substituted aryl or an optionally substituted heteroaryl group having up to 12 carbon atoms, and wherein the optional substituents are each independently selected from; halo, OH, OR⁷, NH₂, NHR⁷, NR⁷R⁷, SH, SR⁷, imidazolyl, R⁷-piperazinyl, morpholino, SO₂R⁷, CF₃, CN, CO₂H, CO₂R⁷, CHO, COR⁷, CONH₂, CONHR⁷, CONR⁷R⁷, and each heteroaryl group contains one or more heteroatoms in its ring system which are each independently selected from O, N or S;
wherein each R⁷ is independently selected from an optionally substituted C₁₋₄ alkyl or an optionally substituted C₂₋₄ alkenyl group and wherein the optional substituents are each independently selected from OH, OR⁸, NH₂, NHR⁸NR⁸₂ or N(OH)R⁸ wherein each R⁸ is independently selected from C₁₋₄ alkyl, C₂₋₄ alkenyl, OH, NO₂, NH₂, CF₃, CN, CO₂H or SH;
D represents up to four of the following groups as substituents at any available ring carbon position; H, R⁹, hydroxy, alkoxy, halogen, NO₂, NH₂, NHR⁹, NR⁹₂, SH, SR⁹, SO₂R⁹, CF₃, CN, CO₂H, CO₂R⁹, CHO, COR⁹, CONH₂, CONHR⁹ or CONR⁹R⁹, cyclic alkylamino, imidazolyl, alkylpiperazinyl and morpholino, wherein each R⁹ independently selected from an optionally substituted C₁₋₄ alkyl or an optionally substituted C₂₋₄ alkenyl group and wherein the optional substituents are each independently selected from OH, OR¹⁰, NH₂, NHR¹⁰, NR¹⁰₂ or N(OH)R¹⁰ wherein each R¹⁰ is independently selected from C₁₋₄ alkyl, C₂₋₄ alkenyl, OH, NO₂ NH₂, CF₃, CN, CO₂H or SH; and wherein any available ring carbon position of formulae II-XVI can also be optionally replaced by —N— when the valency and configuration of the formula allows, the point of attachment of formulae II-XVI to the A group defined above is represented by ♦; and
wherein in formulae XI and XII, m is selected from 2, 3 or 4, and
wherein in formulae XI, XII, XV or XVI J is selected from CH or N; and
wherein in formulae XIII and XIV n is selected from 0, 1 or 2, and
wherein in formulae XV and XVI o is selected from 1 or 2.

32. The compound of Formula I′ as claimed in claim 31 wherein the DNA targeting unit is selected from one of formulae III-IX.

33. The compound of Formula I′ as claimed in claim 31 wherein D of the DNA targeting unit of Formulae II-X is H or Me.

34. The compound of Formula I′ as claimed in claim 30 wherein X is O, NH or CH₂.

35. The compound of Formula I′ as claimed in claim 30 wherein Y₁ represents H.

36. The compound of Formula I′ as claimed in claim 30 wherein A is selected from —(CH₂)₆NH—, —(CH₂)₃NH(CH₂)₃NHCO—, —(CH₂)₃NMe(CH₂)₃NHCO—, —(CH₂)₃NH—, —(CH₂)₂NH(CH₂)₂NHCO— or —(CH₂)₂NMe(CH₂)₂NHCO—.

37. The compound of Formula I′ as claimed in claim 31 wherein X is O—, Y₁ is H, A is —(CH₂)₃NH(CH₂)₃NHCO—, the DNA targeting unit represents formula VI and D is H.

38. The compound of Formula I′ as claimed in claim 31 wherein X is O—, Y₁ is H, A is —(CH₂)₃NMe(CH₂)₃NHCO—, the DNA targeting unit represents formula VI and D is H;

39. The compound of Formula I′ as claimed in claim 31 wherein X is O—, Y₁ is H, A is —(CH₂)₂NH(CH₂)₂NHCO—, the DNA targeting unit represents formula VI and D is H;

40. The compound of Formula I′ as claimed in claim 31 wherein X is O—, Y is H, A is —(CH₂)₂NMe(CH₂)₂NHCO—, the DNA targeting unit represents formula VI and D is H;

41. The compound of Formula I′ as claimed in claim 31 wherein X is O—, Y₁ is H, A is —(CH₂)₃NH(CH₂)₃NHCO—, the DNA targeting unit represents formula VIII and D is H;

42. The compound of Formula I′ as claimed in claim 31 wherein X is O—, Y₁ is H, A is —(CH₂)₃NMe(CH₂)₃NHCO—, the DNA targeting unit represents formula VIII and D is H;

43. The compound of Formula I′ as claimed in claim 31 wherein X is O—, Y₁ is H, A is —(CH₂)₂NH(CH₂)₂NHCO—, the DNA targeting unit represents formula VIII and D is H;

44. The compound of Formula I′ as claimed in claim 31 wherein X is O—, Y₁ is H, A is —(CH₂)₂NMe(CH₂)₂NHCO—, the DNA targeting unit represents formula VIII and D is H;

45. The compound of Formula I′ as claimed in claim 31 wherein X is O—, Y₁ is H, A is —(CH₂)₃NH(CH₂)₃NHCO—, the DNA targeting unit represents formula VIII and D is Me;

46. The compound of Formula I′ as claimed in claim 31 wherein X is O—, Y₁, is H, A is —(CH₂)₃NMe(CH₂)₃NHCO—, the DNA targeting unit represents formula VIII and D is Me;

47. The compound of Formula I′ as claimed in claim 31 X is O—, Y₁ is H, A is —(CH₂)₂NH(CH₂)₂NHCO—, the DNA targeting unit represents formula VIII and D is Me;

48. The compound of Formula I′ as claimed in claim 31 X is O—, Y₁ is H, A is —CH₂)₂NMe(CH₂)₂NHCO—, the DNA targeting unit represents formula VIII and D is Me;

49. The compound of Formula I′ as claimed in claim 31 wherein X is O—, Y₁ is H, A is —(CH₂)₃NH(CH₂)₃NHCO—, the DNA targeting unit represents formula IX and D is Me.

50. The compound of Formula I′ as claimed in claim 31 wherein X is O—, Y₁, is H, A is —(CH₂)₃NMe(CH₂)₃NHCO—, the DNA targeting unit represents formula IX and D is Me;

51. The compound of Formula I′ as claimed in claim 31 wherein X is O—, Y₁ is H, A is —(CH₂)₂NH(CH₂)₂NHCO—, the DNA targeting unit represents formula IX and D is Me;

52. The compound of Formula I′ as claimed in claim 31 wherein X is O—, Y₁, is H, A is —(CH₂)₂NMe(CH₂)₂NHCO—, the DNA targeting unit represents formula XI and D is Me.

53. The compounds of Formula I′ as claimed in claim 30, wherein Y₃ represents CH₃, —CH₂CH₃ or NHCH₂CH₂N(CH₃)₂.

54. A method of therapy for treating cancers including the step of administering a compound of Formula I as defined in claim 1 or a compound of Formula I′ as defined above or a mixture thereof in a therapeutically effective amount to tumour cells in a subject.

55. The method of therapy according to claim 54 wherein the tumour cells are in a hypoxic environment.

56. The method of therapy according to claim 54 further including the step of administering radiotherapy to the tumor cells before, during or after the administration of the compound of Formula I as defined above or a compound of Formula I′ as defined above or a mixture thereof to the tumour cells.

57. The method of therapy according to claim 54 further including the step of administering one or more chemotherapeutic agents to the tumor cells before, during or after the administration of the compound of Formula I as defined above or a compound of Formula I′ as defined above or a mixture thereof to the tumour cells.

58. The method according to claim 54 wherein the therapy can be administered alone or in combination with other chemotherapeutic agents or treatments, either simultaneously or sequentially dependent upon the condition to be treated.

59. The method according to claim 58 wherein the chemotherapeutic treatment is radiation therapy.

60. The method according to claim 59 wherein the chemotherapeutic agents are selected from one or more of: Cisplatin or other platinum-based derivatives, Temozolomide or other DNA methylating agents, Cyclophosphamide or other DNA alkylating agents, Doxorubicin, mitoxantrone, camptothecin or other topoisomerase inhibitors, Methotrexate, gemcitabine or other antimetabolites.

61. A pharmaceutical composition including a therapeutically effective amount of a compound of formula I as claimed in claim 1 or a compound of formula I′ as defined above or a mixture thereof, a pharmaceutically acceptable excipient, adjuvant, carrier, buffer or stabiliser.

62. A method of making a compound of formula XVII 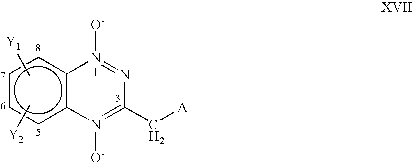
wherein
Y₁ and Y₂ at one or more of the available carbons 5-8 on the benzo ring: are each independently selected from the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino;
wherein each R is independently selected from an optionally substituted C₁₆ alicydic or an optionally substituted C₃₋₆ cyclic alkyl group, and wherein the optional substituents are each independently selected from; halo, OH, OR¹, NO₂ NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR²NR²₂ or N(OH)R² wherein each R² is independently selected from C₁₋₄ alkyl, C₂₋₄ alkenyl, OH, NO₂, NH₂, CF₃, CN, CO₂H or SH, and
A represents an optionally substituted C₁₋₁₂ alkyl group wherein the optional substituents are each independently selected from OH, OR, NH₂, NHR³NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, where each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; or a pharmacologically acceptable salt thereof, including the step of coupling a compound (a) using a palladium reagent to form compound (b) which can then be converted into a compound of XVII as defined above; 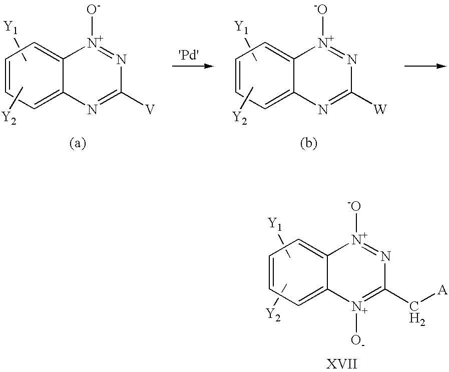
wherein in compound (a)
V is halogen selected from Cl, Br or I and Y₁, Y₂ are as defined above in this claim;
and wherein in compound (b) Y₁, Y₂ are as defined above in this claim, W is selected from an optionally substituted C₁₋₂alkyl, optionally substituted C₂₋₁₂ alkenyl, and optionally substituted C₂₋₁₂alkynyl group, wherein the optional substituents is selected from halo, OH, OR⁶, NO₂, NH₂, NHR⁶NR⁶R⁶SH, SR⁶, imidazolyl, R⁶-piperazinyl, morpholino, SO₂R⁶, CF₃, CN, CO₂H, CO₂R⁶, CHO, COR⁶, CONH₂, CONHR⁶, CONR⁶R⁶, wherein each R⁶ is independently selected from an optionally substituted C₁₋₄ alkyl or an optionally substituted C₂₋₄ alkenyl group and wherein the optional substituents are each independently selected from OH, OR, NH₂, NHR⁷, NR⁷₂ or N(OH)R⁷ wherein each R⁷ is independently selected from C₁₋₄ alkyl, C₂₋₄ alkenyl, OH, NO₂, NH₂, CF₃, CN, CO₂H or SH.

63. A method of making a compound of formula XVII′ 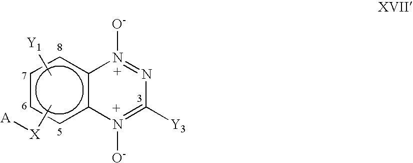
wherein Y₁ represents at one or more of the available carbons 5-8 on the benzo ring the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino; Y₃ is selected from the following groups H, R, OR, NH₂, NHR, NR₂, SO₂R, CF₃, CN, CO₂H, CO₂R, CHO, COR, CONH₂, CONHR or CONRR, cyclic alkylamino, imidazolyl, alkylpiperazinyl and morpholino;
wherein each R of groups Y₁ and Y₃ is independently selected from an optionally substituted C₁₆ alicylic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroazyl group having up to 12 carbon atoms, and wherein the optional substituents are each independently selected from; halo, OH, OR¹, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR²NR²₂ or N(OH)R² wherein each R² is independently selected from C₁₋₄ alkyl, C₂₋₄ alkenyl, OH, NO₂, NH₂, CF₃, CN, CO₂H or SH, and
wherein X represents NH, NMe, CH₂, SO, SO₂, or O;
A represents an optionally substituted C₁₋₁₂ alkyl group wherein the optional substituents are each independently selected from OH, OR, NH₂, NHR³NR³₂, or N(OH)R³ wherein each R³ is independently selected from C₁₋₄ alkyl, C₂₋₄ alkenyl, OH, NO₂ NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, wherein each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂ NH₂, CF₃, CN, CO₂H or SH; and or a pharmacologically acceptable salt thereof;
including the steps of coupling a compound (a) using a palladium reagent to form compound (b) which is then converted into a compound of XVII′ as defined above in this claim; 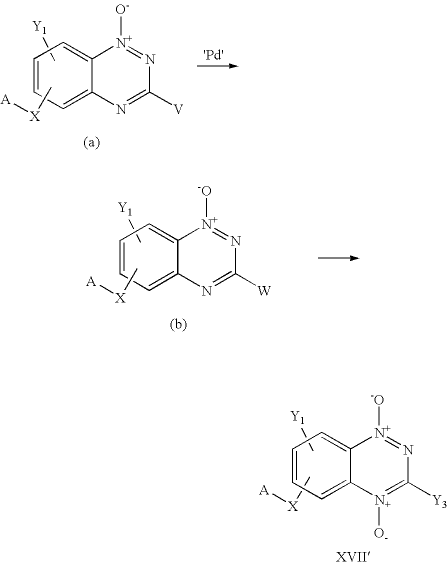
wherein in compound (a) V is halogen which is selected from Cl, Br or I; Y₁, X and A is as defined above in this claim;
and wherein in compound (b) Y₁, X and A are as defined above in this claim, W is selected from an optionally substituted C₁₋₁₂alkyl, optionally substituted C₂₋₁₂alkenyl, and optionally substituted C₂₋₁₂alkynyl group, wherein the optional substituents is selected from halo, OH, ORB, NO₂, NH₂, NHR⁶, NR⁶R⁶, SH, SR⁶, imidazolyl, R⁶-piperazinyl, morpholino, SO₂R⁶, CF₃, CN, CO₂H, CO₂R⁶, CHO, COR⁶, CONH₂, CONHR⁶, CONR⁶R⁶, wherein each R⁶ is independently selected from an optionally substituted C₁₋₄ alkyl or an optionally substituted C₂₋₄ alkenyl group and wherein the optional substituents are each independently selected from OH, OR, NH₂, NHR⁷, NR⁷₂ or N(OH)R⁷ wherein each R⁷ is independently selected from C₁₋₄ alkyl, C₂₋₄ alkenyl, OH, NO₂, NH₂, CF₃, CN, CO₂H or SH.

64. A compound of formula XVIII 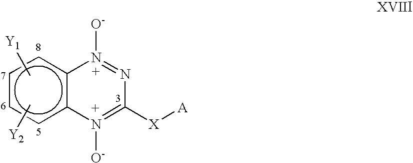
wherein
Y₁ and Y₂ at one or more of the available carbons 5-8 on the benzo ring: are each independently selected from the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and morpholino; wherein each R is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from halo, OH, OR¹, NH₂, NHR¹, NR¹R¹, SH, SR¹ imidazolyl, R¹-piperazinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl, C₂₋₄ alkenyl, OH, NO₂, NH₂, CF₃, CN, CO₂H or SH, and wherein X represents NH, NMe, CH₂, SO, SO₂, or O;
A represents an optionally substituted C₁₋₁₂ alkyl group wherein the optional substituents are each independently selected from OH, OR, NH₂, NHR³, NR³₂, or N(OH)R³ wherein each R³ is independently selected from C₁₋₄ alkyl, C₂₋₄ alkenyl, OH, NO₂, NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, wherein each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵₂ or N(OH)R⁵ wherein each R⁵ is independently selected from C₁₋₄ alkyl, C₂₋₄ alkenyl, OH, NO₂, NH₂, CF₃, CN, CO₂H or SH; or a pharmacologically acceptable salt thereof, with the proviso that:
3-amino 6 or 7-decyl-1,2,4-benzotriazine 1,4 dioxide,
3-(3-N,N-diethylaiminopropylamino-1,2,4-benzotriazine 1,4 dioxide,
7-nitro-3-(2-N,N-diethylaminoethylamino)-1,2,4-benzotriazine 1,4 dioxide,
3-(2-methoxyethyl)-1,2,4-benzotriazine 1,4 dioxide,
3-amino 6 or 7-methoxy-1,2,4-benzotriazine 1,4 dioxide,
N methy, 3-amino-1,2,4-benzotriazine 1,4 dioxide,
3-ethyl-1,2,4-benzotriazine 1,4 dioxide,
3-propyl-1,2,4-benzotriazine 1,4 dioxide and
3-methoxy, 1,2,4-benzotriazine 1,4 dioxide are excluded.

65. A compound of formula XVII′ 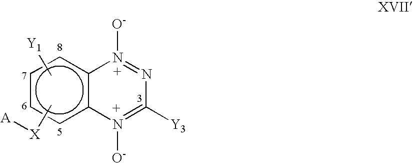
wherein
Y₁ represents at one or more of the available carbons 5-8 on the benzo ring the following groups: halo, H, R, OH, OR, NO₂, NH₂, NHR, NR₂, SH, SR, SO₂R, CF₃, CN, CO₂H, CO₂R, CHO, COR, CONH₂, CONHR or CONRR, cyclic alkylamino, imidazolyl, alkylpiperazinyl and marpholino;
Y₃ is selected from the following groups H, R, OR, NH₂, NHR, NR₂, SO₂R, CF₃, CN, CO₂H, CO₂R, CHO, COR, CONR₂, CONHR or CONRR, cyclic alkylamino, imidazolyl, alkylpiperazinyl and morpholino
wherein each R of groups Y₁ and Y₃ is independently selected from an optionally substituted C₁₋₆ alicyclic or an optionally substituted C₃₋₆ cyclic alkyl group, and wherein the optional substituents are each independently selected from; halo, OH, OR¹, NO₂, NH₂, NHR¹, NR¹R¹, SH, SR¹, imidazolyl, R¹-piperazinyl, morpholino, SO₂R¹, CF₃, CN, CO₂H, CO₂R¹, CHO, COR¹, CONH₂, CONHR¹, CONR¹R¹;
R can also represent an optionally substituted aryl or an optionally substituted heteroaryl group having up to 12 carbon atoms, and wherein the optional substituents are each independently selected from; halo, OH, OR¹, NH₂, NHR¹, NR¹R¹, SH, SR¹, imidazolyl, R¹-piperizinyl, morpholino, SO₂R¹, CF₃, CN, CO₂H, CO₂R¹, CHO, COR¹, CONH₂, CONHR¹, CONR¹R¹, and each heteroaryl group contains one or more heteroatoms in its ring system which are each independently selected from O, N or S;
wherein each R¹ is independently selected from an optionally substituted C₁₋₄ alkyl or an optionally substituted C₂₋₄ alkenyl group and wherein the optional substituents are each independently selected from OH, OR, NH₂, NHR², NR²₂ or N(OH)R² wherein each R² is independently selected from C₁₋₄ alkyl C₂₋₄ alkenyl, OH, NO₂ NH₂, CF₃, CN, CO₂H or SH, and
wherein X represents NH, NMe, CH₂, SO, SO₂, or O;
A represents an optionally substituted C₁₋₁₂ alkyl group wherein the optional substituents are each independently selected from OH, OR, NH₂, NHR³, NR³₂ or N(OH)R³ wherein each R³ is independently selected form C₁₋₄ alkyl, C₂₋₄ alkenyl, OH NO₂ NH₂, CF₃, CN, CO₂H or SH; and wherein the optionally substituted C₁₋₁₂ alkyl chain is optionally interrupted by one or more heteroatom containing linkage moieties selected from O, NH, NR⁴, CONH, CONR⁴, NHCO, NR⁴CO, wherein each R⁴ is independently selected from an optionally substituted C₁₋₄ alkyl or an optionally substituted C₂₋₄ alkenyl group and wherein the optional R⁴ substituents are each independently selected from OH, OR, NH₂, NHR⁵, NR⁵ or N(OH)R⁵ wherein each R⁵ is independently selected from C₁₋₄ alkyl, C₂₋₄ alkenyl, OH, NO₂, NH₂, CF₃, CN, CO₂H or SH; and
wherein X represents NH, NMe, CH₂, SO, SO₂, or O;
or a pharmacologically acceptable salt thereof, with the proviso that:
3-amino 6 or 7-decyl-1,2,4-benzotriazine 1,4 dioxide,
1,2 propanediol 3-[(1,4 dioxide-1,2,4-benzotriazine-7-yl)oxy] are excluded.

66. A method of making a compound of Formula I defined above in claim 1 including the steps of
1 preparing a compound of Formula XVIII as defined above in claim 64; and
2 coupling the compound of Formula XVIII with a DNA targeting agent as defined in claim 2 to provide a compound of Formula I.

67. A method of making a compound of Formula I′ defined in claim 30 including the steps of
1 preparing a compound of Formula XVII′ as defined above; and
2 coupling the compound of Formula XVII′ with a DNA targeting agent as defined above to provide a compound of Formula I′.
